(19) United States
(12) Patent Application Publication
(10) Pub. No.: US 2003/0113891 A1
(43) Pub. Date: Jun. 19, 2003

(54) Method and reagent for the inhibition of NOGO and NOGO receptor genes

(76) Inventors: Lawrence Blatt, Boulder, CO (US); James McSwiggen, Boulder, CO (US); Bharat Chowrira, Broomfield, CO (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 09/827,395

(22) Filed: Apr. 5, 2001

Related U.S. Application Data
(63) Continuation-in-part of application No. 09/780,533, filed on Feb. 9, 2001.
(60) Provisional application No. 60/181,797, filed on Feb. 11, 2000.

(51) Int. Cl. A61K 48/00; C07H 21/02; C12N 9/99

(52) U.S. Cl. 435/184; 536/23.1; 514/44

(57) ABSTRACT

The present invention relates to nucleic acid molecules, including antisense and enzymatic nucleic acid molecules, such as hammerhead ribozymes, DNAzymes, and antisense, which modulate the expression of NOGO and NOGO receptor genes.

This patent application is a continuation-in-part of Blatt, U.S. Ser. No. 09/780,533 filed Feb. 9, 2001, entitled “METHOD AND REAGENT FOR THE INHIBITION OF NOGO GENE” which claims priority from Blatt, U.S. Ser. No. 60/181,797, filed Feb. 11, 2000, entitled “METHOD AND REAGENT FOR THE INHIBITION OF NOGO GENE”. This application is hereby incorporated by reference herein in its entirety including the drawings.

### BACKGROUND OF THE INVENTION

The Sequence Listing file named “MBHB00-878-C Sequence Listing” submitted on Compact Disc-Recordable (CD-R) medium (“010404-_{—}1540”) submitted in duplicate is in compliance with 37 C.F.R. §1.52(e) is incorporated herein by reference.

The present invention provides compounds, compositions, and methods for the study, diagnosis, and treatment of conditions relating to the expression of NOGO and NOGO receptor genes. In particular, the invention provides nucleic acid molecules that are used to modulate the expression of NOGO and NOGO receptor gene products.

The following is a brief description of the current understanding of NOGO and NOGO receptors. The discussion is not meant to be complete and is provided only to assist understanding the invention that follows. The summary is not an admission that any of the work described below is prior art to the claimed invention.

The ceased growth of neurons following development has severe implications for lesions of the central nervous system (CNS) caused by neurodegenerative disorders and traumatic accidents. Although CNS neurons have the capacity to rearrange their axonal and dendritic foci in the developed brain, the regeneration of severed CNS axons spanning distance does not exist. Axonal growth following CNS injury is limited by the local tissue environment rather than intrinsic factors, as indicated by transplantation experiments (Richardson et al., 1980, Nature, 284, 264-265). Non-neuronal glial cells of the CNS, including oligodendrocytes and astrocytes, have been shown to inhibit the axonal growth of dorsal root ganglion neurons in culture (Schwab and Thoenen,1985, J. Neurosci., 5, 2415-2423). Cultured dorsal root ganglion cells can extend their axons across glial cells from the peripheral nervous system, (ie; Schwann cells), but are inhibited by oligodendrocytes and myelin of the CNS (Schwab and Caroni, 1988, J. Neurosci., 8, 2381-2393).

The non-conducive properties of CNS tissue in adult vertebrates is thought to result from the existence of inhibitory factors rather than the lack of growth factors. The identification of proteins with neurite outgrowth inhibitory or repulsive properties include NI-35, NI-250 (Caroni and Schwab, 1988, Neuron, 1, 85-96), myelin-associated glycoprotein (Genbank Accession No M29273), tenascin-R (Genbank Accession No X98085), and NG-2 (Genbank Accession No X61945). Monoclonal antibodies (mAb IN-1) raised against NI-35/250 have been shown to partially neutralize the growth inhibitory effect of CNS myelin and oligodendrocytes. IN-1 treatment in vivo has resulted in long distance fiber regeneration in lesioned adult mammalian CNS tissue (Weibel et al., 1994, Brain Res., 642, 259-266). Additionally, IN-1 treatment in vivo has resulted in the recovery of specific reflex and locomotor functions after spinal cord injury in adult rats (Bregman et al., 1995, Nature, 378, 498-501).

Recently, the cloning of NOGO-A (Genbank Accession No AJ242961), the rat complementary DNA encoding NI-220/250 has been reported (Chen et al., 2000, Nature, 403, 434-439). The NOGO gene encodes at least three major protein products (NOGO-A, NOGO-B, and NOGO-C) resulting from both alternative promoter usage and alternative splicing.

Recombinant NOGO-A inhibits neurite outgrowth from dorsal root ganglia and the spreading of 3T3 firboblasts. Monoclonal antibody IN-1 recognizes NOGO-A and neutralizes NOGO-A inhibition of neuronal growth in vitro. Evidence supports the proposal that NOGO-A is the previously described rat NI-250 since NOGO-A contains all six peptide sequences obtained from purified bNI-220, the bovine equivalent of rat NI-250 (Chen et al supra).

Prinjha et al., 2000, Nature, 403, 383-384, report the cloning of the human NOGO gene which encodes three different NOGO isoforms that are potent inhibitors of neurite outgrowth. Using oligonucleotide primers to amplify and clone overlapping regions of the open reading frame of NOGO cDNA, Phrinjha et al., supra identified three forms of cDNA clone corresponding to the three protein isoforms. The longest ORF of 1,192 amino acids corresponds to NOGO-A (Accession No. AJ251383). An intermediate-length splice variant that lacks residues 186-1,004 corresponds to NOGO-B (Accession No. AJ251384). The shortest splice variant, NOGO-C (Accession No. AJ251385), appears to be the previously described rat vp20 (Accession No. AF051335) and foocen-s (Accession No. AF132048), and also lacks residues 186-1,004. According to Prinjha et al., supra, the NOGO amino-terminal region shows no significant homology to any known protein, while the carboxy-terminal tail shares homology with neuroendocrine-specific proteins and other members of the reticulon gene family. In addition, the carboxy-terminal tail contains a consensus sequence that may serve as an endoplasmic-reticulum retention region. Based on the NOGO protein sequence, Prinjha et al, supra, postulate NOGO to be a membrane associated protein comprising a putative large extracellular domain of 1,024 residues with seven predicted N-linked glycosylation sites, two or three transmembrane domains, and a short carboxy-terminal region of 43 residues.

Grandpre et al., 2000, Nature, also report the identification of NOGO as a potent inhibitor of axon regeneration. The 4.1 kilobase NOGO human cDNA clone identified by Grandpre et al., supra, KIAA0886, is homologous to a cDNA derived from a previous effort to sequence random high molecular-weight brain derived cDNAs (Nagase et al., 1998, DNA Res., 31, 355-364). This cDNA clone encodes a protein that matches all six of the peptide sequences derived from bovine NOGO. Grandpre et al., supra demonstrate that NOGO expression is predominantly associated with the CNS and not the peripheral nervous system (PNS). Cellular localization of NOGO protein appears to be predominantly reticluar in origin, however, NOGO is found on the surface of some oligodentrocytes. An active domain of NOGO has been identified, defined as residues 31-55 of a hydrophilic 66-residue lumenal/extracellular domain. A synthetic fragment corresponding to this sequence exhibits growth-cone collapsing and outgrowth inhibiting activities (Grandpre et al., supra).

A receptor for the NOGO-A extracellular domain (NOGO-66) is described in Fournier et al., 2001, Nature, 409, 341-346. Fournier et al., have shown that isolated NOGO-66 inhibits axonal extension but does not alter non-neuronal cell morphology. The receptor identified has a high affinity for soluble NOGO-66, and is expressed as a glycophosphatidylinostitol-linked protein on the surface of CNS neurons. Furthermore, the expression of the NOGO-66 receptor in neurons that are NOGO insensitive results in NOGO dependent inhibition of axonal growth in these cells. Cleavage of the NOGO-66 receptor and other glycophosphatidylinostitol-linked proteins from axonal surfaces renders neurons insensitive to NOGO-66 inhibition. As such, disruption of the interaction between NOGO-66 and the NOGO-66 receptor provides the possibility of treating a wide variety of neurological diseases, injuries, and conditions.

Hauswirth and Flannery, International PCT Publication No. WO 98/48027, describe materials and methods for the specific expression of proteins in retinal photoreceptor cells consisting of an adeno-associated viral vector contacting a rod or cone-opsin promoter. In addition, ribozymes which degrade mutant mRNA are described for use in the treatment of retinitis pigmentosa.

Fechteler et al., Interanational PCT Publication No. WO 00/03004 describe ribozymes targeting presenilin-2 RNA for the use in treating neurodegenerative diseases such as Alzheimer's disease.

Eldadah et al., 2000, J. Neurosci., 20, 179-186, describe the protection of cerebellar granule cells from apoptosis induced by serum-potassium deprivation from ribozyme mediated inhibition of caspase-3.

Seidman et al., 1999, Antisense Nucleic Acid Drug Dev., 9, 333-340, describe in general terms, the use of antisense and ribozyme constructs for treatment of neurodegenerative diseases.

Denman et al., 1994, Nucleic Acids Research, 22, 2375-82, describe the ribozyme mediated degradation of beta-amyloid peptide precursor mRNA in COS-7 cells.

Schwab and Chen, International PCT publication No. WO 00/31235, describe NOGO proteins and inhibitors of NOGO

### SUMMARY OF THE INVENTION

The invention features novel nucleic acid-based molecules [e.g., enzymatic nucleic acid molecules (ribozymes), antisense nucleic acids, 2-5A antisense chimeras, triplex DNA, decoy RNA, aptamers, antisense nucleic acids containing RNA cleaving chemical groups] and methods to modulate gene expression, for example, genes encoding certain myelin proteins that inhibit or are involved in the inhibition of neurite growth, including axonal regeneration in the CNS. In particular, the instant invention features nucleic-acid based techniques to inhibit the expression of NOGO-A (Accession No. AJ251383), NOGO-B (Accession No. AJ251384), and/or NOGO-C (Accession No. AJ251385), NOGO-66 receptor (Accession No AF283463, Fournier et al, 2001, Nature, 409, 341-346), NI-35, NI-220, and/or NI-250, myelin-associated glycoprotein (Genbank Accession No M29273), tenascin-R (Genbank Accession No X98085), and NG-2 (Genbank Accession No X61945).

In a preferred embodiment, the invention features the use of one or more of the nucleic acid-based techniques independently or in combination to inhibit the expression of the gene(s) encoding NOGO-A, NOGO-B, NOGO-C, NI-35, NI-220, NI-250, myelin-associated glycoprotein, tenascin-R, NG-2 and/or their corresponding receptors. Specifically, the invention features the use of nucleic acid-based techniques to specifically inhibit the expression of NOGO gene (Genbank Accession No. AB020693) and NOGO-66 receptor (Genbank Accession No. AF283463).

The description below of the various aspects and embodiments is provided with reference to the exemplary NOGO-A and NOGO-66 receptor genes. However, the various aspects and embodiments are also directed to other genes which express NOGOA-like inhibitor proteins and other receptors involved in neurite outgrowth inhibition. Those additional genes can be analyzed for target sites using the methods described for NOGO and the NOGO-66 receptor, referred to alternatively as NOGO receptor. Thus, the inhibition and the effects of such inhibition of the other genes can be performed as described herein.

In one embodiment, the invention features the use of an enzymatic nucleic acid molecule, preferably in the hammerhead, NCH, G-cleaver, amberzyme, zinzyme and/or DNAzyme motif, to inhibit the expression of NOGO and/or NOGO receptor genes.

By “inhibit” it is meant that the activity of NOGO or NOGO receptor or level of RNAs or equivalent RNAs encoding one or more protein subunits of NOGO-A, NOGO-B, NOGO-C and/or NOGO receptors is down-regulated or reduced below that observed in the absence of the nucleic acid molecules of the invention. In one embodiment, inhibition with enzymatic nucleic acid molecule preferably is below that level observed in the presence of an enzymatically inactive or attenuated molecule that is able to bind to the same site on the target RNA, but is unable to cleave that RNA. In another embodiment, inhibition with antisense oligonucleotides is preferably below that level observed in the presence of, for example, an oligonucleotide with scrambled sequence or with mismatches. In another embodiment, inhibition of NOGO genes with the nucleic acid molecule of the instant invention is greater in the presence of the nucleic acid molecule than in its absence.

By “enzymatic nucleic acid molecule” it is meant a nucleic acid molecule which has complementarity in a substrate binding region to a specified gene target, and also has an enzymatic activity which is active to specifically cleave target RNA. That is, the enzymatic nucleic acid molecule is able to intermolecularly cleave RNA and thereby inactivate a target RNA molecule. These complementary regions allow sufficient hybridization of the enzymatic nucleic acid molecule to the target RNA and thus permit cleavage. One hundred percent complementarity is preferred, but complementarity as low as 50-75% can also be useful in this invention (see for example Werner and Uhlenbeck, 1995, Nucleic Acids Research, 23, 2092-2096; Hammann et al., 1999, Antisense and Nucleic Acid Drug Dev., 9, 25-31). The nucleic acids can be modified at the base, sugar, and/or phosphate groups. The term enzymatic nucleic acid is used interchangeably with phrases such as ribozymes, catalytic RNA, enzymatic RNA, catalytic DNA, aptazyme or aptamer-binding ribozyme, regulatable ribozyme, catalytic oligonucleotides, nucleozyme, DNAzyme, RNA enzyme, endoribonuclease, endonuclease, minizyme, leadzyme, oligozyme or DNA enzyme. All of these terminologies describe nucleic acid molecules with enzymatic activity. The specific enzymatic nucleic acid molecules described in the instant application are not limiting in the invention and those skilled in the art will recognize that all that is important in an enzymatic nucleic acid molecule of this invention is that it has a specific substrate binding site which is complementary to one or more of the target nucleic acid regions, and that it have nucleotide sequences within or surrounding that substrate binding site which impart a nucleic acid cleaving and/or ligation activity to the molecule (Cech et al., U.S. Pat. No. 4,987,071; Cech et al., 1988, 260 JAMA 3030).

By “nucleic acid molecule” as used herein is meant a molecule having nucleotides. The nucleic acid can be single, double, or multiple stranded and can comprise modified or unmodified nucleotides or non-nucleotides or various mixtures and combinations thereof.

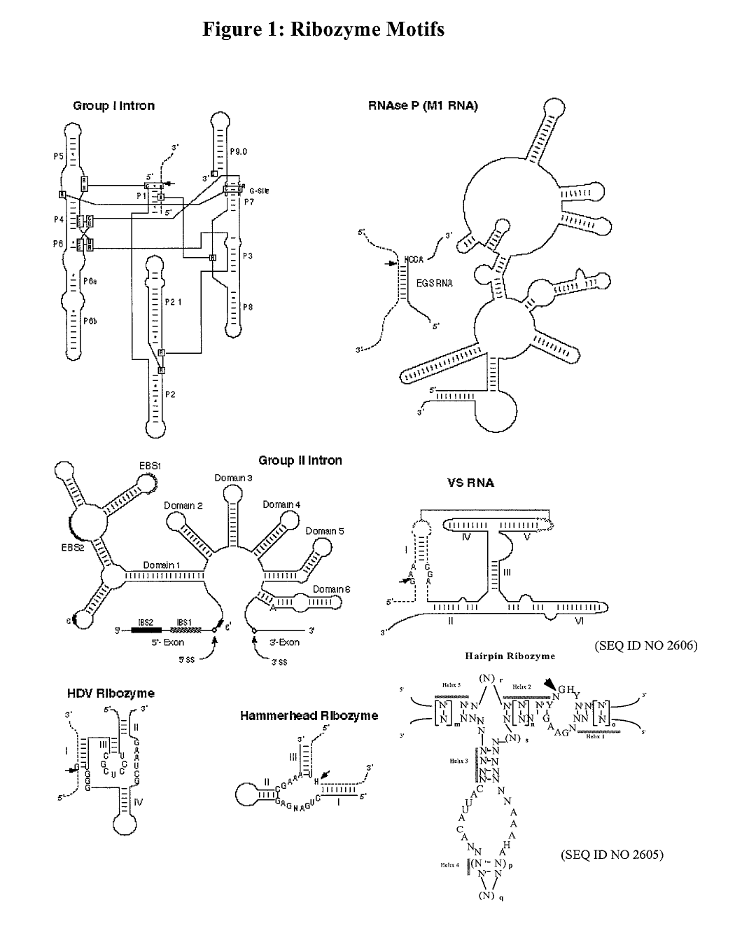

By “enzymatic portion” or “catalytic domain” is meant that portion/region of the enzymatic nucleic acid molecule essential for cleavage of a nucleic acid substrate (for example see FIG. 1).

By “substrate binding arm” or “substrate binding domain” is meant that portion/region of a enzymatic nucleic acid which is able to interact, for example via complementarity (i.e., able to base-pair with), with a portion of its substrate. Preferably, such complementarity is 100%, but can be less if desired. For example, as few as 10 bases out of 14 can be base-paired (see for example Werner and Uhlenbeck, 1995, Nucleic Acids Research, 23, 2092-2096; Hammann et al., 1999, Antisense and Nucleic Acid Drug Dev., 9, 25-31). Examples of such arms are shown generally in FIGS. 1-4. That is, these arms contain sequences within a enzymatic nucleic acid which are intended to bring enzymatic nucleic acid and target RNA together through complementary base-pairing interactions. The enzymatic nucleic acid of the invention can have binding arms that are contiguous or non-contiguous and may be of varying lengths. The length of the binding arm(s) are preferably greater than or equal to four nucleotides and of sufficient length to stably interact with the target RNA; preferably 12-100 nucleotides; more preferably 14-24 nucleotides long (see for example Werner and Uhlenbeck, supra; Hamman et al., supra; Hampel et al., EP0360257; Berzal-Herrance et al., 1993, EMBO J., 12, 2567-73). If two binding arms are chosen, the design is such that the length of the binding arms are symmetrical (i.e., each of the binding arms is of the same length; e.g., five and five nucleotides, or six and six nucleotides, or seven and seven nucleotides long) or asymmetrical (i.e., the binding arms are of different length; e.g., six and three nucleotides; three and six nucleotides long; four and five nucleotides long; four and six nucleotides long; four and seven nucleotides long; and the like).

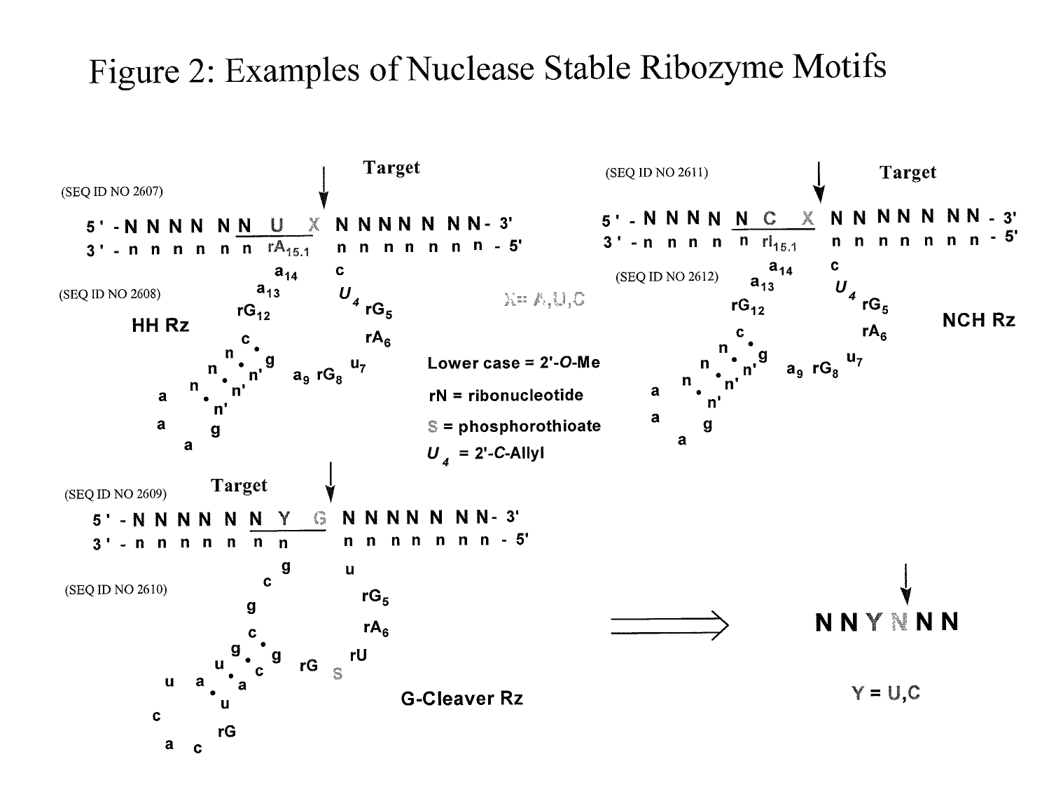

By “Inozyme” or “NCH” motif is meant, an enzymatic nucleic acid molecule comprising a motif as is generally described as NCH Rz in FIG. 2. Inozymes possess endonuclease activity to cleave RNA substrates having a cleavage triplet NCH/, where N is a nucleotide, C is cytidine and H is adenosine, uridine or cytidine, and / represents the cleavage site. H is used interchangeably with X. Inozymes can also possess endonuclease activity to cleave RNA substrates having a cleavage triplet NCN/, where N is a nucleotide, C is cytidine, and / represents the cleavage site. “I” in FIG. 2 represents an Inosine nucleotide, preferably a ribo-Inosine or xylo-Inosine nucleoside.

By “G-cleaver” motif is meant, an enzymatic nucleic acid molecule comprising a motif as is generally described as G-cleaver Rz in FIG. 2. G-cleavers possess endonuclease activity to cleave RNA substrates having a cleavage triplet NYN/, where N is a nucleotide, Y is uridine or cytidine and / represents the cleavage site. G-cleavers can be chemically modified as is generally shown in FIG. 2.

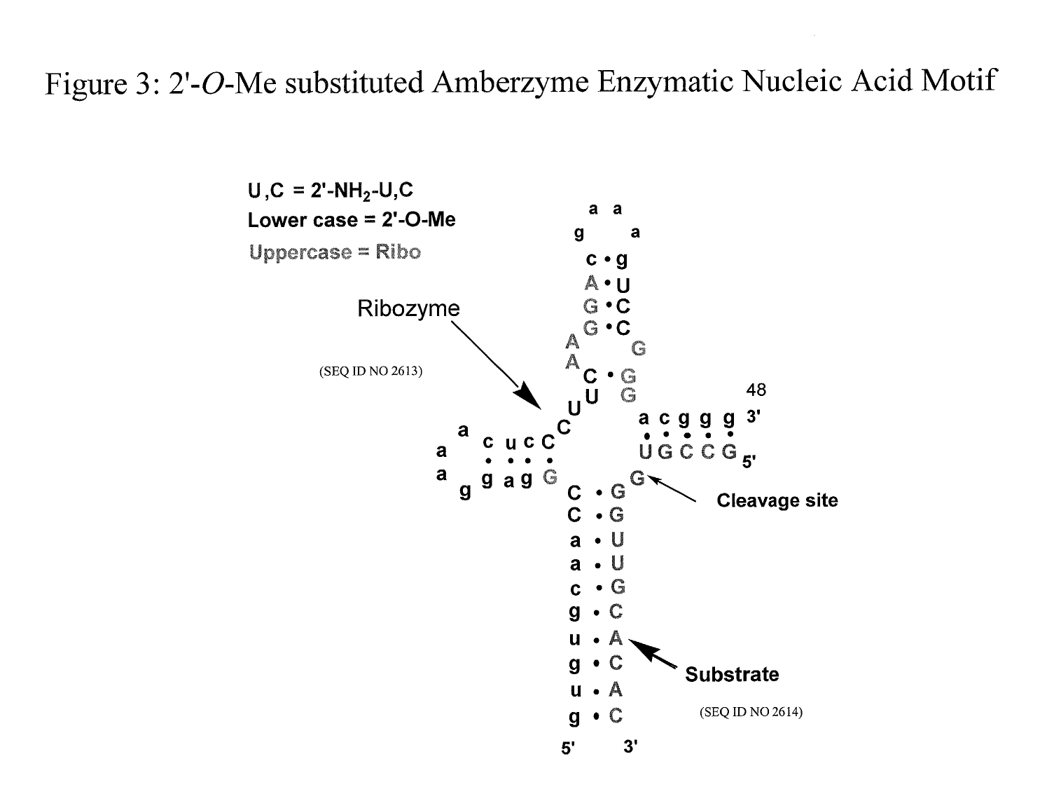

By “amberzyme” motif is meant, an enzymatic nucleic acid molecule comprising a motif as is generally described in FIG. 3. Amberzymes possess endonuclease activity to cleave RNA substrates having a cleavage triplet NG/N, where N is a nucleotide, G is guanosine, and / represents the cleavage site. Amberzymes can be chemically modified to increase nuclease stability through substitutions as are generally shown in FIG. 3. In addition, differing nucleoside and/or non-nucleoside linkers can be used to substitute the 5′-gaaa-3′ loops shown in the figure. Amberzymes represent a non-limiting example of an enzymatic nucleic acid molecule that does not require a ribonucleotide (2′-OH) group within its own nucleic acid sequence for activity.

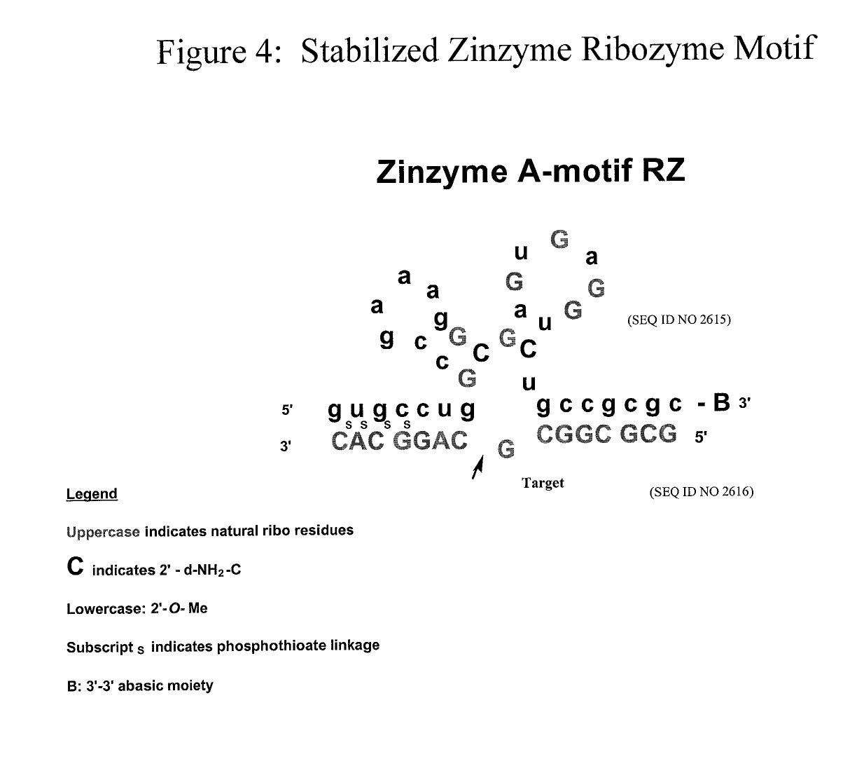

By “zinzyme” motif is meant, an enzymatic nucleic acid molecule comprising a motif as is generally described in FIG. 4. Zinzymes possess endonuclease activity to cleave RNA substrates having a cleavage triplet including but not limited to YG/Y, where Y is uridine or cytidine, and G is guanosine and / represents the cleavage site. Zinzymes can be chemically modified to increase nuclease stability through substitutions as are generally shown in FIG. 4, including substituting 2′-O-methyl guanosine nucleotides for guanosine nucleotides. In addition, differing nucleotide and/or non-nucleotide linkers can be used to substitute the 5′-gaaa-2′ loop shown in the figure. Zinzymes represent a non-limiting example of an enzymatic nucleic acid molecule that does not require a ribonucleotide (2′-OH) group within its own nucleic acid sequence for activity.

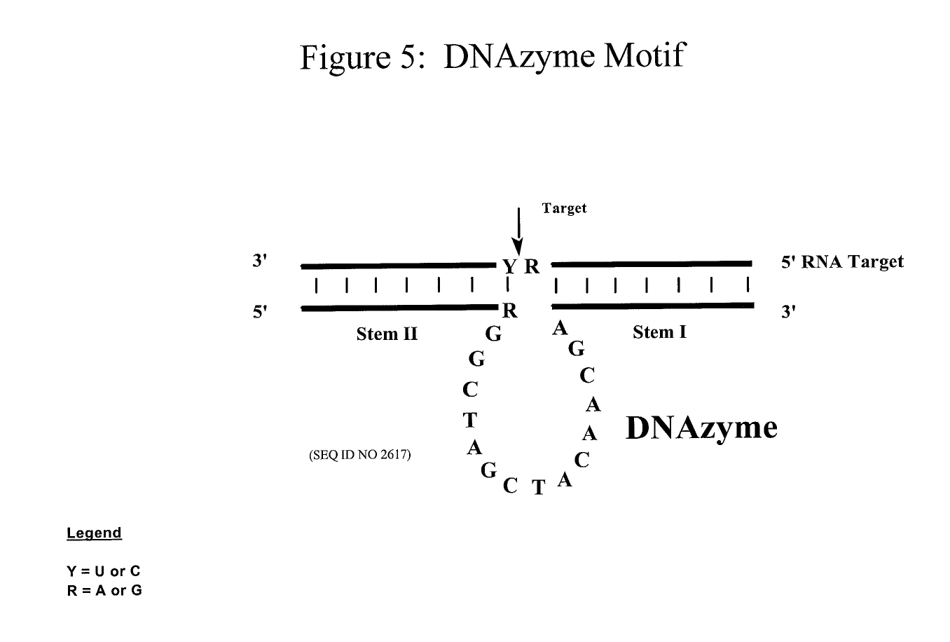

By ‘DNAzyme’ is meant, an enzymatic nucleic acid molecule that does not require the presence of a 2′-OH group for its activity. In particular embodiments the enzymatic nucleic acid molecule can have an attached linker(s) or other attached or associated groups, moieties, or chains containing one or more nucleotides with 2′-OH groups. DNAzymes can be synthesized chemically or expressed endogenously in vivo, by means of a single stranded DNA vector or equivalent thereof. An example of a DNAzyme is shown in FIG. 5 and is generally reviewed in Usman et al., International PCT Publication No. WO 95/11304; Chartrand et al., 1995, NAR 23, 4092; Breaker et al., 1995, Chem. Bio. 2, 655; Santoro et al., 1997, PNAS 94, 4262; Breaker, 1999, Nature Biotechnology, 17, 422-423; and Santoro et. al., 2000, J. Am. Chem. Soc., 122, 2433-39. Additional DNAzyme motifs can be selected for using techniques similar to those described in these references, and hence, are within the scope of the present invention.

By “sufficient length” is meant an oligonucleotide of greater than or equal to 3 nucleotides that is of a length great enough to provide the intended function under the expected condition.

For example, for binding arms of enzymatic nucleic acid “sufficient length” means that the binding arm sequence is long enough to provide stable binding to a target site under the expected binding conditions. Preferably, the binding arms are not so long as to prevent useful turnover of the nucleic acid molecule.

By “stably interact” is meant interaction of the oligonucleotides with target nucleic acid (e.g., by forming hydrogen bonds with complementary nucleotides in the target under physiological conditions) that is sufficient to the intended purpose (e.g., cleavage of target RNA by an enzyme).

By “equivalent” RNA to NOGO is meant to include those naturally occurring RNA molecules having homology (partial or complete) to NOGO-A, NOGO-B, NOGO-C and/or NOGO receptor proteins or encoding for proteins with similar function as NOGO or NOGO receptor proteins in various organisms, including human, rodent, primate, rabbit, pig, protozoans, fungi, plants, and other microorganisms and parasites. The equivalent RNA sequence also includes in addition to the coding region, regions such as 5′-untranslated region, 3′-untranslated region, introns, intron-exon junction and the like.

By “homology” is meant the nucleotide sequence of two or more nucleic acid molecules is partially or completely identical.

By “antisense nucleic acid”, it is meant a non-enzymatic nucleic acid molecule that binds to target RNA by means of RNA-RNA or RNA-DNA or RNA-PNA (protein nucleic acid;

Egholm et al., 1993 Nature 365, 566) interactions and alters the activity of the target RNA (for a review, see Stein and Cheng, 1993 Science 261, 1004 and Woolf et al., U.S. Pat. No. 5,849,902). Typically, antisense molecules are complementary to a target sequence along a single contiguous sequence of the antisense molecule. However, in certain embodiments, an antisense molecule can bind to substrate such that the substrate molecule forms a loop, and/or an antisense molecule can bind such that the antisense molecule forms a loop. Thus, the antisense molecule can be complementary to two (or even more) non-contiguous substrate sequences or two (or even more) non-contiguous sequence portions of an antisense molecule can be complementary to a target sequence or both. For a review of current antisense strategies, see Schmajuk et al., 1999, J. Biol. Chem., 274, 21783-21789, Delihas et al., 1997, Nature, 15, 751-753, Stein et al., 1997, Antisense N. A. Drug Dev., 7, 151, Crooke, 2000, Methods Enzymol., 313, 3-45; Crooke, 1998, Biotech. Genet. Eng. Rev., 15, 121-157, Crooke, 1997, Ad. Pharmacol., 40, 1-49. In addition, antisense DNA can be used to target RNA by means of DNA-RNA interactions, thereby activating RNase H, which digests the target RNA in the duplex. The antisense oligonucleotides can comprise one or more RNAse H activating region, which is capable of activating RNAse H cleavage of a target RNA. Antisense DNA can be synthesized chemically or expressed via the use of a single stranded DNA expression vector or equivalent thereof.

By “RNase H activating region” is meant a region (generally greater than or equal to 4-25 nucleotides in length, preferably from 5-11 nucleotides in length) of a nucleic acid molecule capable of binding to a target RNA to form a non-covalent complex that is recognized by cellular RNase H enzyme (see for example Arrow et al., U.S. Pat. No. 5,849,902; Arrow et al., U.S. Pat. No. 5,989,912). The RNase H enzyme binds to the nucleic acid molecule-target RNA complex and cleaves the target RNA sequence. The RNase H activating region comprises, for example, phosphodiester, phosphorothioate (preferably at least four of the nucleotides are phosphorothiote substitutions; more specifically, 4-11 of the nucleotides are phosphorothiote substitutions); phosphorodithioate, 5′-thiophosphate, or methylphosphonate backbone chemistry or a combination thereof. In addition to one or more backbone chemistries described above, the RNase H activating region can also comprise a variety of sugar chemistries. For example, the RNase H activating region can comprise deoxyribose, arabino, fluoroarabino or a combination thereof, nucleotide sugar chemistry. Those skilled in the art will recognize that the foregoing are non-limiting examples and that any combination of phosphate, sugar and base chemistry of a nucleic acid that supports the activity of RNase H enzyme is within the scope of the definition of the RNase H activating region and the instant invention.

By “2-5A antisense chimera” is meant an antisense oligonucleotide containing a 5′-phosphorylated 2′-5′-linked adenylate residue. These chimeras bind to target RNA in a sequence-specific manner and activate a cellular 2-5A-dependent ribonuclease which, in turn, cleaves the target RNA (Torrence et al., 1993 Proc. Natl. Acad. Sci. USA 90, 1300; Silverman et al., 2000, Methods Enzymol., 313, 522-533; Player and Torrence, 1998, Pharmacol. Ther., 78, 55-113).

By “triplex forming oligonucleotides” is meant an oligonucleotide that can bind to a double-stranded DNA in a sequence-specific manner to form a triple-strand helix. Formation of such triple helix structure has been shown to inhibit transcription of the targeted gene (Duval-Valentin et al., 1992 Proc. Natl. Acad. Sci. USA 89, 504; Fox, 2000, Curr. Med. Chem., 7, 17-37; Praseuth et. al., 2000, Biochim. Biophys. Acta, 1489, 181-206).

By “gene” it is meant a nucleic acid that encodes an RNA, for example, nucleic acid sequences including but not limited to structural genes encoding a polypeptide.

“Complementarity” refers to the ability of a nucleic acid to form hydrogen bond(s) with another RNA sequence by either traditional Watson-Crick or other non-traditional types. In reference to the nucleic molecules of the present invention, the binding free energy for a nucleic acid molecule with its target or complementary sequence is sufficient to allow the relevant function of the nucleic acid to proceed, e.g., enzymatic nucleic acid cleavage, antisense or triple helix inhibition. Determination of binding free energies for nucleic acid molecules is well known in the art (see, e.g., Turner et al., 1987, CSH Symp. Quant. Biol. LII pp.123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

By “RNA” is meant a molecule comprising at least one ribonucleotide residue. By “ribonucleotide” or “2′-OH” is meant a nucleotide with a hydroxyl group at the 2′ position of a β-D-ribo-furanose moiety.

By “decoy RNA” is meant an RNA molecule or aptamer that is designed to preferentially bind to a predetermined ligand. Such binding can result in the inhibition or activation of a target molecule. The decoy RNA or aptamer can compete with a naturally occuring binding target for the binding of a specific ligand. For example, it has been shown that over-expression of HIV trans-activation response (TAR) RNA can act as a “decoy” and efficiently binds HIV tat protein, thereby preventing it from binding to TAR sequences encoded in the HIV RNA (Sullenger et al., 1990, Cell, 63, 601-608). This is but a specific example and those in the art will recognize that other embodiments can be readily generated using techniques generally known in the art, see for example Gold et al., 1995, Annu. Rev. Biochem., 64, 763; Brody and Gold, 2000, J Biotechnol., 74, 5; Sun, 2000, Curr. Opin. Mol. Ther., 2, 100; Kusser, 2000, J. Biotechnol., 74, 27; Hermann and Patel, 2000, Science, 287, 820; and Jayasena, 1999, Clinical Chemistry, 45, 1628. Similarly, a decoy RNA can be designed to bind to a NOGO receptor and block the binding of NOGO or a decoy RNA can be designed to bind to NOGO and prevent interaction with the NOGO receptor.

Several varieties of naturally-occurring enzymatic RNAs are known presently. Each can catalyze the hydrolysis of RNA phosphodiester bonds in trans (and thus can cleave other RNA molecules) under physiological conditions. Table I summarizes some of the characteristics of these ribozymes. In general, enzymatic nucleic acids act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets. Thus, a single ribozyme molecule is able to cleave many molecules of target RNA. In addition, the ribozyme is a highly specific inhibitor of gene expression, with the specificity of inhibition depending not only on the base-pairing mechanism of binding to the target RNA, but also on the mechanism of target RNA cleavage. Single mismatches, or base-substitutions, near the site of cleavage can completely eliminate catalytic activity of a ribozyme.

The enzymatic nucleic acid molecule that cleave the specified sites in NOGO and NOGO receptor-specific RNAs represent a novel therapeutic approach to treat a variety of pathologic indications, including but not limited to CNS injury and cerebrovascular accident (CVA, stroke), Alzheimer's disease, dementia, multiple sclerosis (MS), chemotherapy-induced neuropathy, amyotrophic lateral sclerosis (ALS), Parkinson's disease, ataxia, Huntington's disease, Creutzfeldt-Jakob disease, muscular dystrophy, and/or other neurodegenerative disease states which respond to the modulation of NOGO and NOGO receptor expression.

In one embodiment of the inventions described herein, the enzymatic nucleic acid molecule is formed in a hammerhead or hairpin motif, but can also be formed in the motif of a hepatitis delta virus, group I intron, group II intron or RNase P RNA (in association with an RNA guide sequence), Neurospora VS RNA, DNAzymes, NCH cleaving motifs, or G-cleavers. Examples of such hammerhead motifs are described by Dreyfus, supra, Rossi et al., 1992, AIDS Research and Human Retroviruses 8, 183; of hairpin motifs by Hampel et al., EP0360257, Hampel and Tritz, 1989 Biochemistry 28, 4929, Feldstein et al., 1989, Gene 82, 53, Haseloff and Gerlach, 1989, Gene, 82, 43, and Hampel et al., 1990 Nucleic Acids Res. 18, 299; Chowrira & McSwiggen, U.S. Pat. No. 5,631,359; of the hepatitis delta virus motif is described by Perrotta and Been, 1992 Biochemistry 31, 16; of the RNase P motif by Guerrier-Takada et al., 1983 Cell 35, 849; Forster and Altman, 1990, Science 249, 783; Li and Altman, 1996, Nucleic Acids Res. 24, 835; Neurospora VS RNA ribozyme motif is described by Collins (Saville and Collins, 1990 Cell 61, 685-696; Saville and Collins, 1991 Proc. Natl. Acad. Sci. USA 88, 8826-8830; Collins and Olive, 1993 Biochemistry 32, 2795-2799; Guo and Collins, 1995, EMBO. J 14, 363); Group II introns are described by Griffin et al. 1995, Chem. Biol. 2, 761; Michels and Pyle, 1995, Biochemistry 34, 2965; Pyle et al., International PCT Publication No. WO 96/22689; of the Group I intron by Cech et al., U.S. Pat. No. 4,987,071 and of DNAzymes by Usman et al., International PCT Publication No. WO 95/11304; Chartrand et al., 1995, NAR 23, 4092; Breaker et al., 1995, Chem. Bio. 2, 655; Santoro et al., 1997, PNAS 94, 4262, and Beigelman et al., International PCT publication No. WO 99/55857. NCH cleaving motifs are described in Ludwig & Sproat, International PCT Publication No. WO 98/58058; and G-cleavers are described in Kore et al., 1998, Nucleic Acids Research 26, 4116-4120 and Eckstein et al., International PCT Publication No. WO 99/16871. Additional motifs such as the Aptazyme (Breaker et al., WO 98/43993), Amberzyme (Class I motif; FIG. 3; Beigelman et al., U.S. Ser. No. 09/301,511) and Zinzyme (FIG. 4) (Beigelman et al., U.S. Ser. No. 09/301,511), all included by reference herein including drawings, can also be used in the present invention. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule (Cech et al., U.S. Pat. No. 4,987,071).

In one embodiment of the present invention, a nucleic acid molecule of the instant invention can be between 12 and 100 nucleotides in length. Exemplary enzymatic nucleic acid molecules of the invention are shown in Table III-VII. For example, enzymatic nucleic acid molecules of the invention are preferably between 15 and 50 nucleotides in length, more preferably between 25 and 40 nucleotides in length, e.g., 34, 36, or 38 nucleotides in length (for example see Jarvis et al., 1996, J. Biol. Chem., 271, 29107-29112). Exemplary DNAzymes of the invention are preferably between 15 and 40 nucleotides in length, more preferably between 25 and 35 nucleotides in length, e.g., 29, 30, 31, or 32 nucleotides in length (see for example Santoro et al., 1998, Biochemistry, 37, 13330-13342; Chartrand et al., 1995, Nucleic Acids Research, 23, 4092-4096). Exemplary antisense molecules of the invention are preferably between 15 and 75 nucleotides in length, more preferably between 20 and 35 nucleotides in length, e.g., 25, 26, 27, or 28 nucleotides in length (see for example Woolf et al., 1992, PNAS., 89, 7305-7309; Milner et al., 1997, Nature Biotechnology, 15, 537-541). Exemplary triplex forming oligonucleotide molecules of the invention are preferably between 10 and 40 nucleotides in length, more preferably between 12 and 25 nucleotides in length, e.g., 18, 19, 20, or 21 nucleotides in length (see for example Maher et al., 1990, Biochemistry, 29, 8820-8826; Strobel and Dervan, 1990, Science, 249, 73-75). Those skilled in the art will recognize that all that is required is for the nucleic acid molecule are of length and conformation sufficient and suitable for the nucleic acid molecule to catalyze a reaction contemplated herein. The length of the nucleic acid molecules of the instant invention are not limiting within the general limits stated.

Preferably, a nucleic acid molecule that inhibits NOGO and/or NOGO receptor replication or expression comprises between 12 and 100 bases complementary to a RNA molecule of NOGO or NOGO receptor. Even more preferably, a nucleic acid molecule that inhibits NOGO or NOGO receptor replication or expression comprises between 14 and 24 bases complementary to a RNA molecule of NOGO or NOGO receptor.

In a preferred embodiment the invention provides a method for producing a class of nucleic acid-based gene inhibiting agents which exhibit a high degree of specificity for the RNA of a desired target. For example, the enzymatic nucleic acid molecule is preferably targeted to a highly conserved sequence region of target RNAs encoding NOGO-A, NOGO-B, NOGO-C and/or receptor proteins (specifically NOGO and NOGO receptor genes) such that specific treatment of a disease or condition can be provided with either one or several nucleic acid molecules of the invention. Such nucleic acid molecules can be delivered exogenously to specific tissue or cellular targets as required. Alternatively, the nucleic acid molecules (e.g., ribozymes and antisense) can be expressed from DNA and/or RNA vectors that are delivered to specific cells.

As used in herein “cell” is used in its usual biological sense, and does not refer to an entire multicellular organism. The cell can, for example, be in vitro, e.g., in cell culture, or present in amulticellular organism, including, e.g., birds, plants and mammals such as humans, cows, sheep, apes, monkeys, swine, dogs, and cats. The cell may be prokaryotic (e.g., bacterial cell) or eukaryotic (e.g., mammalian or plant cell).

By “NOGO proteins” is meant, a protein, protein receptor or a mutant protein derivative thereof, comprising neuronal inhibitor activity, preferably CNS neuronal growth inhibitor activity.

By “highly conserved sequence region” is meant, a nucleotide sequence of one or more regions in a target gene does not vary significantly from one generation to the other or from one biological system to the other.

The nucleic acid-based inhibitors of NOGO and NOGO receptor expression are useful for the prevention and/or treatment of diseases and conditions such CNS injury, cerebrovascular accident (CVA, stroke), Alzheimer's disease, dementia, multiple sclerosis (MS), chemotherapy-induced neuropathy, muscular dystrophy and any other diseases or conditions that are related to or will respond to the levels of NOGO and/or NOGO receptor in a cell or tissue, alone or in combination with other therapies. In addition, NOGO and/or NOGO receptor inhibition can be used as a therapeutic target for abrogating CNS neuronal growth inhibition; a situation that can selectively regenerate damaged or lesioned CNS tissue to restore specific reflex and/or locomotor functions.

By “related” is meant that the reduction of NOGO expression (specifically NOGO and/or NOGO receptor gene) RNA levels and thus reduction in the level of the respective protein relieves, to some extent, the symptoms of the disease or condition.

The nucleic acid-based inhibitors of the invention are added directly, or can be complexed with cationic lipids, packaged within liposomes, or otherwise delivered to target cells or tissues. The nucleic acid or nucleic acid complexes can be locally administered to relevant tissues ex vivo, or in vivo through injection or infusion pump, with or without their incorporation in biopolymers. In preferred embodiments, the enzymatic nucleic acid inhibitors comprise sequences, which are complementary to the substrate sequences in Tables III to VII. Examples of such enzymatic nucleic acid molecules also are shown in Tables III to VII. Examples of such enzymatic nucleic acid molecules consist essentially of sequences defined in these tables.

In another embodiment, the invention features antisense nucleic acid molecules and 2-5A chimera including sequences complementary to the substrate sequences shown in Tables III to VII. Such nucleic acid molecules can include sequences as shown for the binding arms of the enzymatic nucleic acid molecules in Tables III to VII. Similarly, triplex molecules can be provided targeted to the corresponding DNA target regions, and containing the DNA equivalent of a target sequence or a sequence complementary to the specified target (substrate) sequence. Typically, antisense molecules are complementary to a target sequence along a single contiguous sequence of the antisense molecule. However, in certain embodiments. an antisense molecule can bind to substrate such that the substrate molecule forms a loop, and/or an antisense molecule can bind such that the antisense molecule forms a loop. Thus, the antisense molecule can be complementary to two (or even more) non-contiguous substrate sequences or two (or even more) non-contiguous sequence portions of an antisense molecule can be complementary to a target sequence or both.

By “consists essentially of” is meant that the active nucleic acid molecule of the invention, for example, an enzymatic nucleic acid molecule, contains an enzymatic center or core equivalent to those in the examples, and binding arms able to hind RNA such that cleavage at the target site occurs. Other sequences can be present which do not interfere with such cleavage. Thus, a core region can, for example, include one or more loop, stem-loop structure, or linker which does not prevent enzymatic activity. Thus, the underlined regions in the sequences in Tables III and IV can be such a loop, stem-loop, nucleotide linker, and/or non-nucleotide linker and can be represented generally as sequence “X”. For example, a core sequence for a hammerhead enzymatic nucleic acid can comprise a conserved sequence, such as 5′-CUGAUGAG-3′ and 5′-CGAA-3′ connected by “X”, where X is 5′-GCCGUUAGGC-3′ (SEQ ID NO 2604), or any other Stem II region known in the art, or a nucleotide and/or non-nucleotide linker. Similarly, for other nucleic acid molecules of the instant invention, such as Inozyme, G-cleaver, amberzyme, zinzyme, DNAzyme, antisense, 2-5A antisense, triplex forming nucleic acid, and decoy nucleic acids, other sequences or non-nucleotide linkers can be present that do not interfere with the function of the nucleic acid molecule.

Sequence X can be a linker of >2 nucleotides in length, preferably 3, 4, 5, 6, 7, 8, 9, 10, 15, 20, 26, 30, where the nucleotides can preferably be internally base-paired to form a stem of preferably >2 base pairs. Alternatively or in addition, sequence X can be a non-nucleotide linker.

In yet another embodiment, the nucleotide linker X can be a nucleic acid aptamer, such as an ATP aptamer, HIV Rev aptamer (RRE), HIV Tat aptamer (TAR) and others (for a review see Gold et al., 1995, Annu. Rev. Biochem., 64, 763; and Szostak & Ellington, 1993, in The RNA World, ed. Gesteland and Atkins, pp. 511, CSH Laboratory Press). A “nucleic acid aptamer” as used herein is meant to indicate a nucleic acid sequence capable of interacting with a ligand. The ligand can be any natural or a synthetic molecule, including but not limited to a resin, metabolites, nucleosides, nucleotides, drugs, toxins, transition state analogs, peptides, lipids, proteins, amino acids, nucleic acid molecules, hormones, carbohydrates, receptors, cells, viruses, bacteria and others.

In yet another embodiment, the non-nucleotide linker X is as defined herein. The term “non-nucleotide” as used herein include either abasic nucleotide, polyether, polyamine, polyamide, peptide, carbohydrate, lipid, or polyhydrocarbon compounds. Specific examples include those described by Seela and Kaiser, Nucleic Acids Res. 1990, 18:6353 and Nucleic Acids Res. 1987, 15:3113; Cload and Schepartz, J. Am. Chem. Soc. 1991, 113:6324; Richardson and Schepartz, J. Am. Chem. Soc. 1991, 113:5109; Ma et al., Nucleic Acids Res. 1993, 21:2585 and Biochemistry 1993, 32:1751; Durand et al., Nucleic Acids Res. 1990, 18:6353; McCurdy et al., Nucleosides & Nucleotides 1991, 10:287; Jschke et al., Tetrahedron Lett. 1993, 34:301; Ono et al., Biochemistry 1991, 30:9914; Arnold et al., International Publication No. WO 89/02439; Usman et al., International Publication No. WO 95/06731; Dudycz et al., International Publication No. WO 95/11910 and Ferentz and Verdine, J. Am. Chem. Soc. 1991, 113:4000, all hereby incorporated by reference herein. A “non-nucleotide” further means any group or compound which can be incorporated into a nucleic acid chain in the place of one or more nucleotide units, including either sugar and/or phosphate substitutions, and allows the remaining bases to exhibit their enzymatic activity. The group or compound can be abasic in that it does not contain a commonly recognized nucleotide base, such as adenosine, guanine, cytosine, uracil or thymine. Thus, in a preferred embodiment, the invention features an enzymatic nucleic acid molecule having one or more non-nucleotide moieties, and having enzymatic activity to cleave an RNA or DNA molecule.

In another aspect of the invention, enzymatic nucleic acid molecules or antisense molecules that interact with target RNA molecules and inhibit NOGO (specifically NOGO and/or NOGO receptor gene) activity are expressed from transcription units inserted into DNA or RNA vectors. The recombinant vectors are preferably DNA plasmids or viral vectors. Enzymatic nucleic acid molecule or antisense expressing viral vectors can be constructed based on, but not limited to, adeno-associated virus, retrovirus, adenovirus, or alphavirus. Preferably, the recombinant vectors capable of expressing the enzymatic nucleic acid molecules or antisense are delivered as described above, and persist in target cells. Alternatively, viral vectors can be used that provide for transient expression of enzymatic nucleic acid molecules or antisense. Such vectors can be repeatedly administered as necessary. Once expressed, the enzymatic nucleic acid molecules or antisense bind to the target RNA and inhibit its function or expression. Delivery of enzymatic nucleic acid molecule or antisense expressing vectors can be systemic, such as by intravenous or intramuscular administration, by administration to target cells ex-planted from the patient followed by reintroduction into the patient, or by any other means that would allow for introduction into the desired target cell. Antisense DNA can be expressed via the use of a single stranded DNA intracellular expression vector.

By “vectors” is meant any nucleic acid- and/or viral-based technique used to deliver a desired nucleic acid.

By “patient” is meant an organism, which is a donor or recipient of explanted cells or the cells themselves. “Patient” also refers to an organism to which the nucleic acid molecules of the invention can be administered. Preferably, a patient is a mammal or mammalian cells. More preferably, a patient is a human or human cells.

By “enhanced enzymatic activity” is meant to include activity measured in cells and/or in vivo where the activity is a reflection of both the catalytic activity and the stability of the nucleic acid molecules of the invention. In this invention, the product of these properties can be increased in vivo compared to an all RNA enzymatic nucleic acid or all DNA enzyme. In some cases, the activity or stability of the nucleic acid molecule can be decreased (i.e., less than ten-fold), but the overall activity of the nucleic acid molecule is enhanced, in vivo.

The nucleic acid molecules of the instant invention, individually, or in combination or in conjunction with other drugs, can be used to treat diseases or conditions discussed above. For example, to treat a disease or condition associated with the levels of NOGO and/or NOGO receptor, the patient can be treated, or other appropriate cells can be treated, as is evident to those skilled in the art, individually or in combination with one or more drugs under conditions suitable for the treatment.

In a further embodiment, the described molecules, such as antisense or ribozymes, can be used in combination with other known treatments to treat conditions or diseases discussed above. For example, the described molecules can be used in combination with one or more known therapeutic agents to treat CNS injury, spinal cord injury, cerebrovascular accident (CVA, stroke), Alzheimer's disease, dementia, multiple sclerosis (MS), chemotherapy-induced neuropathy, amyotrophic lateral sclerosis (ALS), Parkinson's disease, ataxia, Huntington's disease, Creutzfeldt-Jakob disease, muscular dystrophy, and/or other neurodegenerative disease states which respond to the modulation of NOGO and/or NOGO receptor expression.

In another preferred embodiment, the invention features nucleic acid-based inhibitors (e.g., enzymatic nucleic acid molecules (eg; ribozymes), antisense nucleic acids, 2-5A antisense chimeras, triplex DNA, antisense nucleic acids containing RNA cleaving chemical groups) and methods for their use to down regulate or inhibit the expression of genes (e.g., NOGO and/or NOGO receptor) capable of progression and/or maintenance of CNS injury, spinal cord injury, cerebrovascular accident (CVA, stroke), Alzheimer's disease, dementia, multiple sclerosis (MS), chemotherapy-induced neuropathy, amyotrophic lateral sclerosis (ALS), Parkinson's disease, ataxia, Huntington's disease, Creutzfeldt-Jakob disease, muscular dystrophy, and/or other neurodegenerative disease states which respond to the modulation of NOGO and/or NOGO receptor expression.

By “comprising” is meant including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present.

Other features and advantages of the invention will be apparent from the following description of the preferred embodiments thereof, and from the claims.

### DESCRIPTION OF THE PREFERRED EMBODIMENTS

First the drawings will be described briefly.

Drawings

FIG. 1 shows the secondary structure model for seven different classes of enzymatic nucleic acid molecules. Arrow indicates the site of cleavage. --------- indicate the target sequence. Lines interspersed with dots are meant to indicate tertiary interactions. - is meant to indicate base-paired interaction. Group I Intron: P1-P9.0 represent various stem-loop structures (Cech et al., 1994, Nature Struc. Bio., 1, 273). RNase P (M1RNA): EGS represents external guide sequence (Forster et al., 1990, Science, 249, 783; Pace et al., 1990, J. Biol. Chem., 265, 3587). Group II Intron: 5′SS means 5′ splice site; 3′SS means 3′-splice site; IBS means intron binding site; EBS means exon binding site (Pyle et al., 1994, Biochemistry, 33, 2716). VS RNA: I-VI are meant to indicate six stem-loop structures; shaded regions are meant to indicate tertiary interaction (Collins, International PCT Publication No. WO 96/19577). HDV Ribozyme: : I-IV are meant to indicate four stem-loop structures (Been et al., U.S. Pat. No. 5,625,047). Hammerhead Ribozyme: 1-111 are meant to indicate three stem-loop structures; stems I-III can be of any length and can be symmetrical or asymmetrical (Usman et al., 1996, Curr. Op. Struct. Bio., 1, 527). Hairpin Ribozyme: Helix 1, 4 and 5 can be of any length; Helix 2 is between 3 and 8 base-pairs long; Y is a pyrimidine; Helix 2 (H2) is provided with a least 4 base pairs (i.e., n is 1, 2, 3 or 4) and helix 5 can be optionally provided of length 2 or more bases (preferably 3-20 bases, i.e., m is from 1-20 or more). Helix 2 and helix 5 can be covalently linked by one or more bases (i.e., r is >1 base). Helix 1, 4 or 5 can also be extended by 2 or more base pairs (e.g., 4-20 base pairs) to stabilize the ribozyme structure, and preferably is a protein binding site. In each instance, each N and N′ independently is any normal or modified base and each dash represents a potential base-pairing interaction. These nucleotides can be modified at the sugar, base or phosphate. Complete base-pairing is not required in the helices, but is preferred. Helix I and 4 can be of any size (i.e., o and p is each independently from 0 to any number, e.g., 20) as long as some base-pairing is maintained. Essential bases are shown as specific bases in the structure, but those in the art will recognize that one or more can be modified chemically (abasic, base, sugar and/or phosphate modifications) or replaced with another base without significant effect. Helix 4 can be formed from two separate molecules, i.e., without a connecting loop. The connecting loop when present can be a ribonucleotide with or without modifications to its base, sugar or phosphate. “q”≧is 2 bases. The connecting loop can also be replaced with a non-nucleotide linker molecule. H refers to bases A, U, or C. Y refers to pyrimidine bases. “______” refers to a covalent bond. (Burke et al., 1996, Nucleic Acids & Mol. Biol., 10, 129; Chowrira et al., U.S. Pat. No. 5,631,359).

FIG. 2 shows examples of chemically stabilized ribozyme motifs. HH Rz, represents hammerhead ribozyme motif (Usman et al., 1996, Curr. Op. Struct. Bio., 1, 527); NCH Rz represents the NCH ribozyme motif (Ludwig & Sproat, International PCT Publication No. WO 98/58058); G-Cleaver, represents i-cleaver ribozyme motif (Kore et al., 1998, Nucleic Acids Research 26, 4116-4120, Eckstein et al., Internaitional PCT publication No. WO 99/16871). N or n, represent independently a nucleotide which can be same or different and have complementarity to each other; rI, represents ribo-Inosine nucleotide; arrow indicates the site of cleavage within the target. Position 4 of the HH Rz and the NCH Rz is shown as having 2′-C-allyl modification, but those skilled in the art will recognize that this position can be modified with other modifications well known in the art, so long as such modifications do not significantly inhibit the activity of the ribozyme.

FIG. 3 shows an example of the Amberzyme ribozyme motif that is chemically stabilized (see for example Beigelman et al., International PCT publication No. WO 99/55857).

FIG. 4 shows an example of the Zinzyme A ribozyme motif that is chemically stabilized (see for example Beigelman et al., Beigelman et al., International PCT publication No. WO 99/55857).

FIG. 5 shows an example of a DNAzyme motif described by Santoro et al., 1997, PNAS, 94, 4262.

Mechanism of Action of Nucleic Acid Molecules of the Invention

Antisense:

Antisense molecules can be modified or unmodified RNA, DNA, or mixed polymer oligonucleotides and primarily function by specifically binding to matching sequences resulting in inhibition of peptide synthesis (Wu-Pong, November 1994, BioPharm, 20-33). The antisense oligonucleotide binds to target RNA by Watson Crick base-pairing and blocks gene expression by preventing ribosomal translation of the bound sequences either by steric blocking or by activating RNase H enzyme. Antisense molecules can also alter protein synthesis by interfering with RNA processing or transport from the nucleus into the cytoplasm (Mukhopadhyay & Roth, 1996, Crit. Rev. in Oncogenesis 7, 151-190).

In addition, binding of single stranded DNA to RNA can result in nuclease degradation of the heteroduplex (Wu-Pong, supra; Crooke, supra). To date, the only backbone modified DNA chemistry which will act as substrates for RNase H are phosphorothioates, phosphorodithioates, and borontrifluoridates. Recently it has been reported that 2′-arabino and 2′-fluoro arabino-containing oligos can also activate RNase H activity.

A number of antisense molecules have been described that utilize novel configurations of chemically modified nucleotides, secondary structure, and/or RNase H substrate domains (Woolf et al., International PCT Publication No. WO 98/13526; Thompson et al., International PCT Publication No. WO 99/54459; Hartmann et al., U.S. Ser. No. 60/101,174 which was filed on Sep. 21, 1998) all of these are incorporated by reference herein in their entirety.

In addition, antisense deoxyoligoribonucleotides can be used to target RNA by means of DNA-RNA interactions, thereby activating RNase H, which digests the target RNA in the duplex. Antisense DNA can be expressed via the use of a single stranded DNA intracellular expression vector or equivalents and variations thereof.

Triplex Forming Oligonucleotides (TFO):

Single stranded DNA can be designed to bind to genomic DNA in a sequence specific manner. TFOs are comprised of pyrimidine-rich oligonucleotides which bind DNA helices through Hoogsteen Base-pairing (Wu-Pong, supra). The resulting triple helix composed of the DNA sense, DNA antisense, and TFO disrupts RNA synthesis by RNA polymerase. The TFO mechanism can result in gene expression or cell death since binding can be irreversible (Mukhopadhyay & Roth, supra).

2-5A Antisense Chimera:

The 2-5A system is an interferon mediated mechanism for RNA degradation found in higher vertebrates (Mitra et al., 1996, Proc Nat Acad Sci USA 93, 6780-6785). Two types of enzymes, 2-5A synthetase and RNase L, are required for RNA cleavage. The 2-5A synthetases require double stranded RNA to form 2′-5′ oligoadenylates (2-5A). 2-5A then acts as an allosteric effector for utilizing RNase L which has the ability to cleave single stranded RNA. The ability to form 2-5A structures with double stranded RNA makes this system particularly useful for inhibition of viral replication.

(2′-5′) oligoadenylate structures can be covalently linked to antisense molecules to form chimeric oligonucleotides capable of RNA cleavage (Torrence, supra). These molecules putatively bind and activate a 2-5A dependent RNase, the oligonucleotide/enzyme complex then binds to a target RNA molecule which can then be cleaved by the RNase enzyme.

Enzymatic Nucleic Acid:

Several varieties of naturally-occurring enzymatic RNAs are presently known. In addition, several in vitro selection (evolution) strategies (Orgel, 1979, Proc. R. Soc. London, B 205, 435) have been used to evolve new nucleic acid catalysts capable of catalyzing cleavage and ligation of phosphodiester linkages (Joyce, 1989, Gene, 82, 83-87; Beaudry et al., 1992, Science 257, 635-641; Joyce, 1992, Scientific American 267, 90-97; Breaker et al., 1994, TIBTECH 12, 268; Bartel et al., 1993, Science 261:1411-1418; Szostak, 1993, TIBS 17, 89-93; Kumar et al., 1995, FASEB J., 9, 1183; Breaker, 1996, Curr. Op. Biotech., 7, 442; Santoro et al, 1997, Proc. Natl. Acad. Sci., 94, 4262; Tang et al., 1997, RNA 3, 914; Nakamaye & Eckstein, 1994, supra; Long & Uhlenbeck, 1994, supra; Ishizaka et al., 1995, supra; Vaish et al., 1997, Biochemistry 36, 6495; all of these are incorporated by reference herein). Each can catalyze a series of reactions including the hydrolysis of phosphodiester bonds in trans (and thus can cleave other RNA molecules) under physiological conditions.

Nucleic acid molecules of this invention will block to some extent NOGO-A, NOGO-B, and/or NOGO-C protein expression and can be used to treat disease or diagnose disease associated with the levels of NOGO-A, NOGO-B, and/or NOGO-C.

The enzymatic nature of an enzymatic nucleic acid molecule has significant advantages, one advantage being that the concentration of enzymatic nucleic acid molecule necessary to affect a therapeutic treatment is lower. This advantage reflects the ability of the enzymatic nucleic acid molecule to act enzymatically. Thus, a single enzymatic nucleic acid molecule molecule is able to cleave many molecules of target RNA. In addition, the enzymatic nucleic acid molecule is a highly specific inhibitor, with the specificity of inhibition depending not only on the base-pairing mechanism of binding to the target RNA, but also on the mechanism of target RNA cleavage. Single mismatches, or base-substitutions, near the site of cleavage can be chosen to completely eliminate catalytic activity of a enzymatic nucleic acid molecule.

Nucleic acid molecules having an endonuclease enzymatic activity are able to repeatedly cleave other separate RNA molecules in a nucleotide base sequence-specific manner. Such enzymatic nucleic acid molecules can be targeted to virtually any RNA transcript, and achieved efficient cleavage in vitro (Zaug et al., 324, Nature 429 1986; Uhlenbeck, 1987 Nature 328, 596; Kim et al., 84 Proc. Natl. Acad. Sci. USA 8788, 1987; Dreyfus, 1988, Einstein Quart. J. Bio. Med., 6, 92; Haseloff and Gerlach, 334 Nature 585, 1988; Cech, 260 JAMA 3030, 1988; and Jefferies et al., 17 Nucleic Acids Research 1371, 1989; Santoro et al., 1997 supra).

Because of their sequence specificity, trans-cleaving enzymatic nucleic acid molecules can be used as therapeutic agents for human disease (Usman & McSwiggen, 1995 Ann. Rep. Med. Chem. 30, 285-294; Christoffersen and Marr, 1995 J. Med. Chem. 38, 2023-2037). Enzymatic nucleic acid molecules can be designed to cleave specific RNA targets within the background of cellular RNA. Such a cleavage event renders the RNA non-functional and abrogates protein expression from that RNA. In this manner, synthesis of a protein associated with a disease state can be selectively inhibited (Warashina et al., 1999, Chemistry and Biology, 6, 237-250.

The nucleic acid molecules of the instant invention are also referred to as GeneBloc reagents, which are essentially nucleic acid molecules (eg; ribozymes, antisense) capable of down-regulating gene expression.

Target Sites

Targets for useful enzymatic nucleic acid molecules and antisense nucleic acids can be determined as disclosed in Draper et al., WO 93/23569; Sullivan et al., WO 93/23057; Thompson et al., WO 94/02595; Draper et al., WO 95/04818; McSwiggen et al., U.S. Pat. No. 5,525,468, and hereby incorporated by reference herein in totality. Other examples include the following PCT applications, which concern inactivation of expression of disease-related genes: WO 95/23225, WO 95/13380, WO 94/02595, incorporated by reference herein. Rather than repeat the guidance provided in those documents here, below are provided specific examples of such methods, not limiting to those in the art. Enzymatic nucleic acid molecules and antisense to such targets are designed as described in those applications and synthesized to be tested in vitro and in vivo, as also described. The sequences of human NOGO RNAs were screened for optimal enzymatic nucleic acid and antisense target sites using a computer-folding algorithm. Antisense, hammerhead, DNAzyme, NCH, amberzyme, zinzyme, or G-Cleaver enzymatic nucleic acid molecule binding/cleavage sites were identified. These sites are shown in Tables III to VII (all sequences are 5′ to 3′ in the tables; underlined regions can be any sequence “X” or linker X, the actual sequence is not relevant here). The nucleotide base position is noted in the Tables as that site to be cleaved by the designated type of enzymatic nucleic acid molecule. While human sequences can be screened and enzymatic nucleic acid molecule and/or antisense thereafter designed, as discussed in Stinchcomb et al., WO 95/23225, mouse targeted enzymatic nucleic acid molecules can be useful to test efficacy of action of the enzymatic nucleic acid molecule and/or antisense prior to testing in humans.

Antisense, hammerhead, DNAzyme, NCH, amberzyme, zinzyme or G-Cleaver enzymatic nucleic acid molecule binding/cleavage sites were identified. The nucleic acid molecules are individually analyzed by computer folding (Jaeger et al., 1989 Proc. Natl. Acad. Sci. USA, 86, 7706) to assess whether the sequences fold into the appropriate secondary structure. Those nucleic acid molecules with unfavorable intramolecular interactions such as between the binding arms and the catalytic core are eliminated from consideration. Varying binding arm lengths can be chosen to optimize activity.

Antisense, hammerhead, DNAzyme, NCH, amberzyme, zinzyme or G-Cleaver enzymatic nucleic acid molecule binding/cleavage sites were identified and were designed to anneal to various sites in the RNA target. The binding arms are complementary to the target site sequences described above. The nucleic acid molecules were chemically synthesized. The method of synthesis used follows the procedure for normal DNA/RNA synthesis as described below and in Usman et al., 1987 J. Am. Chem. Soc., 109, 7845; Scaringe et al., 1990 Nucleic Acids Res., 18, 5433; and Wincott et al., 1995 Nucleic Acids Res. 23, 2677-2684; Caruthers et al., 1992, Methods in Enzymology 211,3-19.

Synthesis of Nucleic Acid Molecules

Synthesis of nucleic acids greater than 100 nucleotides in length is difficult using automated methods, and the therapeutic cost of such molecules is prohibitive. In this invention, small nucleic acid motifs (“small refers to nucleic acid motifs less than about 100 nucleotides in length, preferably less than about 80 nucleotides in length, and more preferably less than about 50 nucleotides in length; e.g., antisense oligonucleotides, hammerhead or the NCH ribozymes) are preferably used for exogenous delivery. The simple structure of these molecules increases the ability of the nucleic acid to invade targeted regions of RNA structure. Exemplary molecules of the instant invention are chemically synthesized, and others can similarly be synthesized.

Oligonucleotides (eg; antisense GeneBlocs) are synthesized using protocols known in the art as described in Caruthers et al., 1992, Methods in Enzymology 211, 3-19, Thompson et al., International PCT Publication No. WO 99/54459, Wincott et al., 1995, Nucleic Acids Res. 23, 2677-2684, Wincott et al., 1997, Methods Mol. Bio., 74, 59, Brennan et al., 1998, Biotechnol Bioeng., 61, 33-45, and Brennan, U.S. Pat. No. 6,001,311. All of these references are incorporated herein by reference. The synthesis of oligonucleotides makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 2.5 min coupling step for 2′-O-methylated nucleotides and a 45 sec coupling step for 2′-deoxy nucleotides. Table II outlines the amounts and the contact times of the reagents used in the synthesis cycle. Alternatively, syntheses at the 0.2 μmol scale can be performed on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 105-fold excess of S-ethyl tetrazole (60 μL of 0.25 M=15 μmol) can be used in each coupling cycle of 2′-O-methyl residues relative to polymer-bound 5′-hydroxyl. A 22-fold excess (40 μL of 0.11 M=4.4 μmol) of deoxy phosphoramidite and a 70-fold excess of S-ethyl tetrazole (40 μL of 0.25 M=10 μmol) can be used in each coupling cycle of deoxy residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include; detritylation solution is 3% TCA in methylene chloride (ABI); capping is performed with 16% N-methyl imidazole in THF (ABI) and 10% acetic anhydride/10% 2,6-lutidine in THF (ABI); and oxidation solution is 16.9 mM 12, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide, 0.05 M in acetonitrile) is used.

Deprotection of the antisense oligonucleotides is performed as follows: the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H2O/3:1:1, vortexed and the supernatant is then added to the first supernatant. The combined supernatants, containing the oligoribonucleotide, are dried to a white powder.

The method of synthesis used for normal RNA including certain enzymatic nucleic acid molecules follows the procedure as described in Usman et al., 1987, J. Am. Chem. Soc., 109, 7845; Scaringe et al., 1990, Nucleic Acids Res., 18, 5433; and Wincott et al., 1995, Nucleic Acids Res. 23, 2677-2684 Wincott et al., 1997, Methods Mol. Bio., 74, 59, and makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 7.5 min coupling step for alkylsilyl protected nucleotides and a 2.5 min coupling step for 2′-O-methylated nucleotides. Table II outlines the amounts and the contact times of the reagents used in the synthesis cycle. Alternatively, syntheses at the 0.2 μmol scale can be done on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 75-fold excess of S-ethyl tetrazole (60 μL of 0.25 M=15 μmol) can be used in each coupling cycle of 2′-O-methyl residues relative to polymer-bound 5′-hydroxyl. A 66-fold excess (120 μL of 0.11 M=13.2 μmol) of alkylsilyl (ribo) protected phosphoramidite and a 150-fold excess of S-ethyl tetrazole (120 μL of 0.25 M=30 μmol) can be used in each coupling cycle of ribo residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include; detritylation solution is 3% TCA in methylene chloride (ABI); capping is performed with 16% N-methyl imidazole in THF (ABI) and 10% acetic anhydride/10% 2,6-lutidine in THF (ABI); oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide0.05 M in acetonitrile) is used.

Deprotection of the RNA is performed using either a two-pot or one-pot protocol. For the two-pot protocol, the polymer-bound trityl-on oligoribonucleotide is transferred to a 4 mL glass screw top vial and suspended in a solution of 40% aq. methylamine (1 mL) at 65° C. for 10 min.

After cooling to −20° C., the supernatant is removed from the polymer support. The support is washed three times with 1.0 mL of EtOH:MeCN:H2O/3:1:1, vortexed and the supernatant is then added to the first supernatant. The combined supernatants, containing the oligoribonucleotide, are dried to a white powder. The base deprotected oligoribonucleotide is resuspended in anhydrous TEA/HF/NMP solution (300 μL of a solution of 1.5 mL N-methylpyrrolidinone, 750 μL TEA and 1 mL TEA·3HF to provide a 1.4 M HF concentration) and heated to 65° C. After 1.5 h, the oligomer is quenched with 1.5 M NH₄HCO₃.

Alternatively, for the one-pot protocol, the polymer-bound trityl-on oligoribonucleotide is transferred to a 4 mL glass screw top vial and suspended in a solution of 33% ethanolic methylamine/DMSO: 1/1 (0.8 mL) at 65° C. for 15 min. The vial is brought to r.t. TEA-3HF (0.1 mL) is added and the vial is heated at 65° C. for 15 min. The sample is cooled at −20° C. and then quenched with 1.5 M NH₄HCO₃.

For purification of the trityl-on oligomers, the quenched NH₄HCO₃ solution is loaded onto a C-18 containing cartridge that had been prewashed with acetonitrile followed by 50 mM TEAA. After washing the loaded cartridge with water, the RNA is detritylated with 0.5% TFA for 13 min. The cartridge is then washed again with water, salt exchanged with 1 M NaCl and washed with water again. The oligonucleotide is then eluted with 30% acetonitrile.

Inactive hammerhead ribozymes or binding attenuated control (BAC) oligonucleotides) are synthesized by substituting a U for G₅ and a U for A₁₄ (numbering from Hertel, K. J., et al, 1992, Nucleic Acids Res., 20, 3252). Similarly, one or more nucleotide substitutions can be introduced in other enzymatic nucleic acid molecules to inactivate the molecule and such molecules can serve as a negative control.

The average stepwise coupling yields are typically >98% (Wincott et al., 1995 Nucleic Acids Res. 23, 2677-2684). Those of ordinary skill in the art will recognize that the scale of synthesis can be adapted to be larger or smaller than the example described above including but not limited to 96 well format, all that is important is the ratio of chemicals used in the reaction.

Alternatively, the nucleic acid molecules of the present invention can be synthesized separately and joined together post-synthetically, for example by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

The nucleic acid molecules of the present invention are modified extensively to enhance stability by modification with nuclease resistant groups, for example, 2′-amino, 2′-C-allyl, 2′-flouro, 2′-O-methyl, 2′-H (for a review see Usman and Cedergren, 1992, TIBS 17, 34; Usman et al., 1994, Nucleic Acids Symp. Ser. 31, 163). Ribozymes are purified by gel electrophoresis using general methods or are purified by high pressure liquid chromatography (HPLC; See Wincott et al., Supra, the totality of which is hereby incorporated herein by reference) and are re-suspended in water.

The sequences of the ribozymes that are chemically synthesized, are shown in Tables III to VII. The sequences of the antisense constructs that are chemically synthesized, are complementary to the Substrate sequences shown in Tables III to VII. Those in the art will recognize that these sequences are representative only of many more such sequences where the enzymatic portion of the ribozyme (all but the binding arms) is altered to affect activity. The ribozyme and antisense construct sequences listed in Tables III to VII can be formed of ribonucleotides or other nucleotides or non-nucleotides. Such ribozymes with enzymatic activity are equivalent to the ribozymes described specifically in the Tables.

Optimizing Activity of the Nucleic Acid Molecule of the Invention.

Chemically synthesizing nucleic acid molecules with modifications (base, sugar and/or phosphate) that prevent their degradation by serum ribonucleases can increase their potency (see e.g., Eckstein et al., International Publication No. WO 92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; Sproat, U.S. Pat. No. 5,334,711; and Burgin et al, supra; all of these describe various chemical modifications that can be made to the base, phosphate and/or sugar moieties of the nucleic acid molecules herein). Modifications which enhance their efficacy in cells, and removal of bases from nucleic acid molecules to shorten oligonucleotide synthesis times and reduce chemical requirements are desired. (All these publications are hereby incorporated by reference herein).

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-flouro, 2′-O-methyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry , 35, 14090). Sugar modification of nucleic acid molecules have been extensively described in the art (see Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci., 1992, 17, 334-339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. Ser. No. 60/082,404 which was filed on Apr. 20, 1998; Karpeisky et al., 1998, Tetrahedron Lett., 39, 1131; Earnshaw and Gait, 1998, Biopolymers (Nucleic acid Sciences), 48, 39-55; Verma and Eckstein, 1998, Annu. Rev. Biochem., 67, 99-134; and Burlina et al., 1997, Bioorg. Med. Chem., 5, 1999-2010; all of the references are hereby incorporated in their totality by reference herein). Such publications describe general methods and strategies to determine the location of incorporation of sugar, base and/or phosphate modifications and the like into ribozymes without inhibiting catalysis, and are incorporated by reference herein. In view of such teachings, similar modifications can be used as described herein to modify the nucleic acid molecules of the instant invention.

While chemical modification of oligonucleotide internucleotide linkages with phosphorothioate, phosphorothioate, and/or 5′-methylphosphonate linkages improves stability, too many of these modifications can cause some toxicity. Therefore when designing nucleic acid molecules the amount of these internucleotide linkages should be minimized. The reduction in the concentration of these linkages should lower toxicity resulting in increased efficacy and higher specificity of these molecules.

Nucleic acid molecules having chemical modifications which maintain or enhance activity are provided. Such nucleic acid is also generally more resistant to nucleases than unmodified nucleic acid. Thus, in a cell and/or in in vivo the activity may not be significantly lowered. Therapeutic nucleic acid molecules delivered exogenously are optimally stable within cells until translation of the target RNA has been inhibited long enough to reduce the levels of the undesirable protein. This period of time varies between hours to days depending upon the disease state. Clearly, nucleic acid molecules must be resistant to nucleases in order to function as effective intracellular therapeutic agents. Improvements in the chemical synthesis of RNA and DNA (Wincott et al., 1995 Nucleic Acids Res. 23, 2677; Caruthers et al., 1992, Methods in Enzymology 211,3-19 (incorporated by reference herein) have expanded the ability to modify nucleic acid molecules by introducing nucleotide modifications to enhance their nuclease stability as described above.

Use of the nucleic acid-based molecules of the invention can lead to better treatment of the disease progression by affording the possibility of combination therapies (e.g., multiple antisense or enzymatic nucleic acid molecules targeted to different genes, nucleic acid molecules coupled with known small molecule inhibitors, or intermittent treatment with combinations of molecules (including different motifs) and/or other chemical or biological molecules). The treatment of patients with nucleic acid molecules can also include combinations of different types of nucleic acid molecules.

Therapeutic nucleic acid molecules (e.g. enzymatic nucleic acid molecules and antisense nucleic acid molecules) delivered exogenously are optimally stable within cells until translation of the target RNA has been inhibited long enough to reduce the levels of the undesirable protein. This period of time varies between hours to days depending upon the disease state. These nucleic acid molecules should be resistant to nucleases in order to function as effective intracellular therapeutic agents. Improvements in the chemical synthesis of nucleic acid molecules described in the instant invention and in the art have expanded the ability to modify nucleic acid molecules by introducing nucleotide modifications to enhance their nuclease stability as described above.

In another embodiment, nucleic acid catalysts having chemical modifications which maintain or enhance enzymatic activity are provided. Such nucleic acids are also generally more resistant to nucleases than unmodified nucleic acid. Thus, in a cell and/or in vivo the activity of the nucleic acid may not be significantly lowered. As exemplified herein such enzymatic nucleic acids are useful in a cell and/or in vivo even if activity over all is reduced 10 fold (Burgin et al., 1996, Biochemistry, 35, 14090). Such enzymatic nucleic acids herein are said to “maintain” the enzymatic activity of an all RNA ribozyme or all DNA DNAzyme.

In another aspect the nucleic acid molecules comprise a 5′ and/or a 3′-cap structure.

By “cap structure” is meant chemical modifications, which have been incorporated at either terminus of the oligonucleotide (see for example Wincott et al., WO 97/26270, incorporated by reference herein). These terminal modifications protect the nucleic acid molecule from exonuclease degradation, and can help in delivery and/or localization within a cell. The cap can be present at the 5′-terminus (5′-cap) or at the 3′-terminus (3′-cap) or can be present on both terminus. In non-limiting examples, the 5′-cap includes inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Wincott et al., International PCT publication No. WO 97/26270, incorporated by reference herein).

In another embodiment the 3′-cap includes, for example 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Iyer, 1993, Tetrahedron 49, 1925; incorporated by reference herein).

By the term “non-nucleotide” is meant any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine.

An “alkyl” group refers to a saturated aliphatic hydrocarbon, including straight-chain, branched-chain, and cyclic alkyl groups. Preferably, the alkyl group has 1 to 12 carbons. More preferably it is a lower alkyl of from 1 to 7 carbons, more preferably 1 to 4 carbons. The alkyl group can be substituted or unsubstituted. When substituted the substituted group(s) is preferably, hydroxyl, cyano, alkoxy, ═O, ═S, NO2 or N(CH3)2, amino, or SH. The term also includes alkenyl groups which are unsaturated hydrocarbon groups containing at least one carbon-carbon double bond, including straight-chain, branched-chain, and cyclic groups. Preferably, the alkenyl group has 1 to 12 carbons. More preferably it is a lower alkenyl of from 1 to 7 carbons, more preferably 1 to 4 carbons. The alkenyl group can be substituted or unsubstituted. When substituted the substituted group(s) is preferably, hydroxyl, cyano, alkoxy, ═O, ═S, NO2, halogen, N(CH3)2, amino, or SH. The term “alkyl” also includes alkynyl groups which have an unsaturated hydrocarbon group containing at least one carbon-carbon triple bond, including straight-chain, branched-chain, and cyclic groups. Preferably, the alkynyl group has 1 to 12 carbons. More preferably it is a lower alkynyl of from 1 to 7 carbons, more preferably 1 to 4 carbons. The alkynyl group can be substituted or unsubstituted. When substituted the substituted group(s) is preferably, hydroxyl, cyano, alkoxy, ═O, ═S, NO2 or N(CH3)2, amino or SH.

Such alkyl groups can also include aryl, alkylaryl, carbocyclic aryl, heterocyclic aryl, amide and ester groups. An “aryl” group refers to an aromatic group which has at least one ring having a conjugated p electron system and includes carbocyclic aryl, heterocyclic aryl and biaryl groups, all of which can be optionally substituted. The preferred substituent(s) of aryl groups are halogen, trihalomethyl, hydroxyl, SH, OH, cyano, alkoxy, alkyl, alkenyl, alkynyl, and amino groups. An “alkylaryl” group refers to an alkyl group (as described above) covalently joined to an aryl group (as described above). Carbocyclic aryl groups are groups wherein the ring atoms on the aromatic ring are all carbon atoms. The carbon atoms are optionally substituted. Heterocyclic aryl groups are groups having from 1 to 3 heteroatoms as ring atoms in the aromatic ring and the remainder of the ring atoms are carbon atoms. Suitable heteroatoms include oxygen, sulfur, and nitrogen, and include furanyl, thienyl, pyridyl, pyrrolyl, N-lower alkyl pyrrolo, pyrimidyl, pyrazinyl, imidazolyl and the like, all optionally substituted. An “amide” refers to an —C(O)—NH—R, where R is either alkyl, aryl, alkylaryl or hydrogen. An “ester” refers to an —C(O)—OR′, where R is either alkyl, aryl, alkylaryl or hydrogen.

By “nucleotide” is meant a heterocyclic nitrogenous base in N-glycosidic linkage with a phosphorylated sugar. Nucleotides are recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, for example, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

By “nucleoside” is meant a heterocyclic nitrogenous base in N-glycosidic linkage with a sugar. Nucleosides are recognized in the art to include natural bases (standard), and modified bases well known in the art. Such bases are generally located at the 1′ position of a nucleoside sugar moiety. Nucleosides generally comprise a base and sugar group. The nucleosides can be unmodified or modified at the sugar, and/or base moiety, (also referred to interchangeably as nucleoside analogs, modified nucleosides, non-natural nucleosides, non-standard nucleosides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, -D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleoside bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

In one embodiment, the invention features modified enzymatic nucleic acid molecules with phosphate backbone modifications comprising one or more phosphorothioate, phosphorodithioate, methylphosphonate, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications see Hunziker and Leumann, 1995, Nucleic Acid Analogues: Synthesis and Properties, in Modern Synthetic Methods, VCH, 331-417, and Mesmacker et al., 1994, Novel Backbone Replacements for Oligonucleotides, in Carbohydrate Modifications in Antisense Research, ACS, 24-39. These references are hereby incorporated by reference herein.

By “abasic” is meant sugar moieties lacking a base or having other chemical groups in place of a base at the 1′ position, (for more details see Wincott et al., International PCT publication No. WO 97/26270).

By “unmodified nucleoside” is meant one of the bases adenine, cytosine, guanine, thymine, uracil joined to the 1′ carbon of P-D-ribo-furanose.

By “modified nucleoside” is meant any nucleotide base which contains a modification in the chemical structure of an unmodified nucleotide base, sugar and/or phosphate.

In connection with 2′-modified nucleotides as described for the present invention, by “amino” is meant 2′-NH2 or 2′-O—NH2, which can be modified or unmodified. Such modified groups are described, for example, in Eckstein et al., U.S. Pat. No. 5,672,695 and Matulic-Adamic et al., WO 98/28317, respectively, which are both incorporated by reference in their entireties.

Various modifications to nucleic acid (e.g., antisense and ribozyme) structure can be made to enhance the utility of these molecules. For example, such modifications can enhance shelf-life, half-life in vitro, stability, and ease of introduction of such oligonucleotides to the target site, including e.g., enhancing penetration of cellular membranes and confering the ability to recognize and bind to targeted cells.

Use of these molecules can lead to better treatment of the disease progression by affording the possibility of combination therapies (e.g., multiple enzymatic nucleic acid molecules targeted to different genes, enzymatic nucleic acid molecules coupled with known small molecule inhibitors, or intermittent treatment with combinations of enzymatic nucleic acid molecules (including different enzymatic nucleic acid molecule motifs) and/or other chemical or biological molecules). The treatment of patients with nucleic acid molecules can also include combinations of different types of nucleic acid molecules. Therapies can be devised which include a mixture of enzymatic nucleic acid molecules (including different enzymatic nucleic acid molecule motifs), antisense and/or 2-5A chimera molecules to one or more targets to alleviate symptoms of a disease.

Administration of Nucleic Acid Molecules

Methods for the delivery of nucleic acid molecules are described in Akhtar et al., 1992, Trends Cell Bio., 2, 139; and Delivery Strategies for Antisense Oligonucleotide Therapeutics, ed. Akhtar, 1995 which are both incorporated herein by reference. Sullivan et al., PCT WO 94/02595, further describes the general methods for delivery of enzymatic RNA molecules.

These protocols can be utilized for the delivery of virtually any nucleic acid molecule. Nucleic acid molecules can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. Alternatively, the nucleic acid/vehicle combination is locally delivered by direct injection or by use of an infusion pump. Many examples in the art describe CNS delivery methods of oligonucleotides by osmotic pump, (see Chun et al., 1998, Neuroscience Letters, 257, 135-138, D'Aldin et al., 1998, Mol. Brain Research, 55, 151-164, Dryden et al., 1998, J. Endocrinol., 157, 169-175, Ghirnikar et al., 1998, Neuroscience Letters, 247, 21-24) or direct infusion (Broaddus et al, 1997, Neurosurg. Focus, 3, article 4). Other routes of delivery include, but are not limited to oral (tablet or pill form) and/or intrathecal delivery (Gold, 1997, Neuroscience, 76, 1153-1158). For a comprehensive review on drug delivery strategies including broad coverage of CNS delivery, see Ho et al., 1999, Curr. Opin. Mol. Ther., 1, 336-343 and Jain, Drug Delivery Systems: Technologies and Commercial Opportunities, Decision Resources, 1998 and Groothuis et al., 1997, J. NeuroVirol., 3, 387-400. More detailed descriptions of nucleic acid delivery and administration are provided in Sullivan et al., supra, Draper et al., PCT WO3/23569, Beigelman et al., PCT WO99/05094, and Klimuk et al., PCT WO99/04819 all of which have been incorporated by reference herein.

Experiments have demonstrated the efficient in vivo uptake of nucleic acids by neurons. As an example of local administration of nucleic acids to nerve cells, Sommer et al., 1998, Antisense Nuc. Acid Drug Dev., 8, 75, describe a study in which a 15mer phosphorothioate antisense nucleic acid molecule to c-fos is administered to rats via microinjection into the brain. Antisense molecules labeled with tetramethylrhodamine-isothiocyanate (TRITC) or fluorescein isothiocyanate (FITC) were taken up by exclusively by neurons thirty minutes post-injection. A diffuse cytoplasmic staining and nuclear staining was observed in these cells. As an example of systemic administration of nucleic acid to nerve cells, Epa et al., 2000, Antisense Nuc. Acid Drug Dev., 10, 469, describe an in vivo mouse study in which beta-cyclodextrin-adamantane-oligonucleotide conjugates were used to target the p75 neurotrophin receptor in neuronally differentiated PC12 cells. Following a two week course of IP administration, pronounced uptake of p75 neurotrophin receptor antisense was observed in dorsal root ganglion (DRG) cells. In addition, a marked and consistent down-regulation of p75 was observed in DRG neurons. Additional approaches to the targeting of nucleic acid to neurons are described in Broaddus et al., 1998, J. Neurosurg., 88(4), 734; Karle et al., 1997, Eur. J. Pharmocol., 340(2/3), 153; Bannai et al., 1998, Brain Research, 784(1,2), 304; Rajakumar et al., 1997, Synapse, 26(3), 199; Wu-pong et al., 1999, BioPharm, 12(1), 32; Bannai et al., 1998, Brain Res. Protoc., 3(1), 83; Simantov et al., 1996, Neuroscience, 74(1), 39. Nucleic acid molecules of the invention are therefore amenable to delivery to and uptake by cells that express NOGO and NOGO receptors for modulation of NOGO and/or NOGO receptor expression.

The delivery of nucleic acid molecules of the invention, targeting NOGO and NOGO receptors is provided by a variety of different strategies. Traditional approaches to CNS delivery that can be used include, but are not limited to, intrathecal and intracerebroventricular administration, implantation of catheters and pumps, direct injection or perfusion at the site of injury or lesion, injection into the brain arterial system, or by chemical or osmotic opening of the blood-brain barrier. Other approaches can include the use of various transport and carrier systems, for example though the use of conjugates and biodegradable polymers. Furthermore, gene therapy approaches, for example as described in Kaplitt et al., U.S. Pat. No. 6,180,613, can be used to express nucleic acid molecules in the CNS.

The molecules of the instant invention can be used as pharmaceutical agents. Pharmaceutical agents prevent, inhibit the occurrence, or treat (alleviate a symptom to some extent, preferably all of the symptoms) of a disease state in a patient.

The negatively charged polynucleotides of the invention can be administered (e.g., RNA, DNA or protein) and introduced into a patient by any standard means, with or without stabilizers, buffers, and the like, to form a pharmaceutical composition. When it is desired to use a liposome delivery mechanism, standard protocols for formation of liposomes can be followed. The compositions of the present invention can also be formulated and used as tablets, capsules or elixirs for oral administration; suppositories for rectal administration; sterile solutions; suspensions for injectable administration; and the other compositions known in the art.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or patient, preferably a human. Suitable forms, in part, depend upon the use or the route of entry, for example oral, transdermal, or by injection. Such forms should not prevent the composition or formulation from reaching a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect.

By “systemic administration” is meant in vivo systemic absorption or accumulation of drugs in the blood stream followed by distribution throughout the entire body. Administration routes which lead to systemic absorption include, without limitations: intravenous, subcutaneous, intraperitoneal, inhalation, oral, intrapulmonary and intramuscular. Each of these administration routes expose the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation which can facilitate the association of drug with the surface of cells, such as, lymphocytes and macrophages is also useful. This approach can provide enhanced delivery of the drug to target cells by taking advantage of the specificity of macrophage and lymphocyte immune recognition of abnormal cells, such as cancer cells. By pharmaceutically acceptable formulation is meant, a composition or formulation that allows for the effective distribution of the nucleic acid molecules of the instant invention in the physical location most suitable for their desired activity. Non-limiting examples of agents suitable for formulation with the nucleic acid molecules of the instant invention include: P-glycoprotein inhibitors (such as Pluronic P85) which can enhance entry of drugs into the CNS (Jolliet-Riant and Tillement, 1999, Fundam. Clin. Pharmacol., 13, 16-26); biodegradable polymers, such as poly (DL-lactide-coglycolide) microspheres for sustained release delivery after intracerebral implantation (Emerich, D F et al, 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). Other non-limiting examples of delivery strategies, including CNS delivery of the nucleic acid molecules of the instant invention include material described in Boado et al., 1998, J. Pharm. Sci., 87, 1308-1315; Tyler et al., 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058. All these references are hereby incorporated herein by reference.

The invention also features the use of the composition comprising surface-modified liposomes containing poly (ethylene glycol) lipids (PEG-modified, or long-circulating liposomes or stealth liposomes). These formulations offer a method for increasing the accumulation of drugs in target tissues. This class of drug carriers resists opsonization and elimination by the mononuclear phagocytic system (MPS or RES). thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al., 1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392; all of which are incorporated by reference herein). Long-circulating liposomes are also likely to protect drugs from nuclease degradation to a greater extent compared to cationic liposomes, based on their ability to avoid accumulation in metabolically aggressive MPS tissues such as the liver and spleen. All of these references are incorporated by reference herein.

The present invention also includes compositions prepared for storage or administration which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985) hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 100 mg/kg body weight/day of active ingredients is administered dependent upon potency of the negatively charged polymer.

The nucleic acid molecules of the present invention can also be administered to a patient in combination with other therapeutic compounds to increase the overall therapeutic effect. The use of multiple compounds to treat an indication can increase the beneficial effects while reducing the presence of side effects.

Alternatively, certain of the nucleic acid molecules of the instant invention can be expressed within cells from eukaryotic promoters (e.g., Izant and Weintraub, 1985, Science, 229, 345; McGarry and Lindquist, 1986, Proc. Natl. Acad. Sci., USA 83, 399; Scanlon et al., 1991, Proc. Natl. Acad. Sci. USA, 88, 10591-5; Kashani-Sabet et al., 1992, Antisense Res. Dev., 2, 3-15; Dropulic et al, 1992, J. Virol., 66, 1432-41; Weerasinghe et al., 1991, J. Virol., 65, 5531-4; Ojwang et al., 1992, Proc. Natl. Acad. Sci. USA, 89, 10802-6; Chen et al., 1992, Nucleic Acids Res., 20, 4581-9; Sarver et al., 1990 Science, 247, 1222-1225; Thompson et al., 1995, Nucleic Acids Res., 23, 2259; Good et al., 1997, Gene Therapy, 4, 45; all of these references are hereby incorporated in their totalities by reference herein). Those skilled in the art realize that any nucleic acid can be expressed in eukaryotic cells from the appropriate DNA/RNA vector. The activity of such nucleic acids can be augmented by their release from the primary transcript by a enzymatic nucleic acid (Draper et al., PCT WO 93/23569, and Sullivan et al, PCT WO 94/02595; Ohkawa et al., 1992, Nucleic Acids Symp. Ser., 27, 15-6; Taira et al., 1991, Nucleic Acids Res., 19, 5125-30; Ventura et al., 1993, Nucleic Acids Res., 21, 3249-55; Chowrira et al., 1994, J. Biol. Chem., 269, 25856; all of these references are hereby incorporated in their totalities by reference herein). Gene therapy approaches specific to the CNS are described by Blesch et al., 2000, Drug News Perspect., 13, 269-280; Peterson et al., 2000, Cent. Nerv. Syst. Dis., 485-508; Peel and Klein, 2000, J. Neurosci. Methods, 98, 95-104; Hagihara et al., 2000, Gene Ther., 7, 759-763; and Herrlinger et al, 2000, Methods Mol. Med., 35, 287-312. AAV-mediated delivery of nucleic acid to cells of the nervous system is further described by Kaplitt et al., U.S. Pat. No. 6,180,613.

In another aspect of the invention, RNA molecules of the present invention are preferably expressed from transcription units (see for example Couture et al., 1996, TIG., 12, 510) inserted into DNA or RNA vectors. The recombinant vectors are preferably DNA plasmids or viral vectors. Ribozyme expressing viral vectors can be constructed based on, but not limited to, adeno-associated virus, retrovirus, adenovirus, or alphavirus. Preferably, the recombinant vectors capable of expressing the nucleic acid molecules are delivered as described above, and persist in target cells. Alternatively, viral vectors can be used that provide for transient expression of nucleic acid molecules. Such vectors can be repeatedly administered as necessary. Once expressed, the nucleic acid molecule binds to the target mRNA. Delivery of nucleic acid molecule expressing vectors can be systemic, such as by intravenous or intramuscular administration, by administration to target cells ex-planted from the patient followed by reintroduction into the patient, or by any other means that would allow for introduction into the desired target cell (for a review see Couture et al., 1996, TIG., 12, 510).

In one aspect the invention features an expression vector comprising a nucleic acid sequence encoding at least one of the nucleic acid molecules of the instant invention is disclosed. The nucleic acid sequence encoding the nucleic acid molecule of the instant invention is operable linked in a manner which allows expression of that nucleic acid molecule.

In another aspect the invention features an expression vector comprising: a) a transcription initiation region (e.g., eukaryotic pol I, II or III initiation region); b) a transcription termination region (e.g., eukaryotic pol I, II or III termination region); c) a nucleic acid sequence encoding at least one of the nucleic acid catalyst of the instant invention; and wherein said sequence is operably linked to said initiation region and said termination region, in a manner which allows expression and/or delivery of said nucleic acid molecule. The vector can optionally include an open reading frame (ORF) for a protein operably linked on the 5′ side or the 3′-side of the sequence encoding the nucleic acid catalyst of the invention; and/or an intron (intervening sequences).

Transcription of the nucleic acid molecule sequences are driven from a promoter for eukaryotic RNA polymerase I (pol I), RNA polymerase II (pol II), or RNA polymerase III (pol III). Transcripts from pol II or pol III promoters are expressed at high levels in all cells; the levels of a given pol II promoter in a given cell type depends on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. U S A, 87, 6743-7; Gao and Huang 1993, Nucleic Acids Res., 21, 2867-72; Lieber et al., 1993, Methods Enzymol., 217, 47-66; Zhou et al., 1990, Mol. Cell. Biol., 10, 4529-37). All of these references are incorporated by reference herein. Several investigators have demonstrated that nucleic acid molecules, such as ribozymes expressed from such promoters can function in mammalian cells (e.g. Kashani-Sabet et al., 1992, Antisense Res. Dev., 2, 3-15; Ojwang et al. 1992, Proc. Natl. Acad. Sci. U S A, 89, 10802-6; Chen et al, 1992, Nucleic Acids Res., 20, 4581-9; Yu et al., 1993, Proc. Natl. Acad. Sci. U S A, 90, 6340-4; L'Huillier et al., 1992, EMBO J., 11, 4411-8; Lisziewicz et al., 1993, Proc. Natl. Acad. Sci. U. S. A, 90, 8000-4; Thompson et al., 1995, Nucleic Acids Res., 23, 2259; Sullenger & Cech, 1993, Science, 262, 1566). More specifically, transcription units such as the ones derived from genes encoding U6 small nuclear (snRNA), transfer RNA (tRNA) and adenovirus VA RNA are useful in generating high concentrations of desired RNA molecules such as ribozymes in cells (Thompson et al., supra; Couture and Stinchcomb, 1996, supra; Noonberg et al., 1994, Nucleic Acid Res., 22, 2830; Noonberg et al., U.S. Pat. No. 5,624,803; Good et al., 1997, Gene Ther., 4, 45; Beigelman et al., International PCT Publication No. WO 96/18736; all of these publications are incorporated by reference herein. The above ribozyme transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (for a review see Couture and Stinchcomb, 1996, supra).

In another aspect the invention features an expression vector comprising nucleic acid sequence encoding at least one of the nucleic acid molecules of the invention, in a manner which allows expression of that nucleic acid molecule. The expression vector comprises in one embodiment; a) a transcription initiation region; b) a transcription termination region; c) a nucleic acid sequence encoding at least one said nucleic acid molecule; and wherein said sequence is operably linked to said initiation region and said termination region, in a manner which allows expression and/or delivery of said nucleic acid molecule.

In another embodiment the expression vector comprises: a) a transcription initiation region; b) a transcription termination region; c) an open reading frame; d) a nucleic acid sequence encoding at least one said nucleic acid molecule, wheren said sequence is operably linked to the 3′-end of said open reading frame; and wherein said sequence is operably linked to said initiation region, said open reading frame and said termination region, in a manner which allows expression and/or delivery of said nucleic acid molecule. In yet another embodiment the expression vector comprises: a) a transcription initiation region; b) a transcription termination region; c) an intron; d) a nucleic acid sequence encoding at least one said nucleic acid molecule; and wherein said sequence is operably linked to said initiation region, said intron and said termination region, in a manner which allows expression and/or delivery of said nucleic acid molecule.

In another embodiment, the expression vector comprises: a) a transcription initiation region; b) a transcription termination region; c) an intron; d) an open reading frame; e) a nucleic acid sequence encoding at least one said nucleic acid molecule, wherein said sequence is operably linked to the 3′-end of said open reading frame; and wherein said sequence is operably linked to said initiation region, said intron, said open reading frame and said termination region, in a manner which allows expression and/or delivery of said nucleic acid molecule.

### EXAMPLES

The following are non-limiting examples showing the selection, isolation, synthesis and activity of nucleic acids of the instant invention.

The following examples demonstrate the selection and design of Antisense, hammerhead, DNAzyme, NCH, Amberzyme, Zinzyme, or G-Cleaver ribozyme molecules and binding/cleavage sites within NOGO and NOGO receptor RNA.

Nucleic Acid Inhibition of NOGO and NOGO Receptor Target RNA

The lack of axon regeneration capacity in the adult CNS manifests as a limiting factor in the treatment of CNS injury, cerebrovascular accident (CVA, stroke), chemotherapy-induced neuropathy, and possibly in neurodegenerative diseases such as Alzheimer's disease, dementia, multiple sclerosis (MS), chemotherapy-induced neuropathy, amyotrophic lateral sclerosis (ALS), Parkinson's disease, ataxia, Huntington's disease, Creutzfeldt-Jakob disease, and/or muscular dystrophy. Neuron growth inhibition results from physical barriers imposed by glial scars, a lack of neurotrophic factors, and growth-inhibitory molecules associated with myelin. The abrogation of neurite growth inhibition creates the potential to treat conditions for which there is currently no definitive medical intervention. The inhibition of NOGO (Genbank Accession No AB020693) and NOGO-66 receptor (Genbank Accession No. AF283463) is demonstrated in the following examples.

### Example 1

### Identification of Potential Target Sites in Human NOGO RNA

The sequence of human NOGO and NOGO receptor genes are screened for accessible sites using a computer-folding algorithm. Regions of the RNA that do not form secondary folding structures and contained potential enzymatic nucleic acid molecule and/or antisense binding/cleavage sites are identified. The sequences of these binding/cleavage sites are shown in Tables III-VII.

### Example 2

### Selection of Enzymatic Nucleic Acid Cleavage Sites in Human NOGO and NOGO Receptor RNA

Enzymatic nucleic acid molecule target sites are chosen by analyzing sequences of Human NOGO (Genbank accession No: AB020693) and prioritizing the sites on the basis of folding. Enzymatic nucleic acid molecules are designed that can bind each target and are individually analyzed by computer folding (Christoffersen et al., 1994 J. Mol Struc. Theochem, 311, 273; Jaeger et al., 1989, Proc. Natl. Acad. Sci. USA, 86, 7706) to assess whether the enzymatic nucleic acid molecule sequences fold into the appropriate secondary structure. Those enzymatic nucleic acid molecules with unfavorable intramolecular interactions between the binding arms and the catalytic core are eliminated from consideration. As noted below, varying binding arm lengths can be chosen to optimize activity. Generally, at least 5 bases on each arm are able to bind to, or otherwise interact with, the target RNA.

### Example 3

### Chemical Synthesis and Purification of Ribozymes and Antisense for Efficient Cleavage and/or Blocking of NOGO and NOGO Receptor RNA

Enzymatic nucleic acid molecules and antisense constructs are designed to anneal to various sites in the RNA message. The binding arms of the enzymatic nucleic acid molecules are complementary to the target site sequences described above, while the antisense constructs are fully complimentary to the target site sequences described above. The enzymatic nucleic acid molecules and antisense constructs were chemically synthesized. The method of synthesis used followed the procedure for normal RNA synthesis as described above and in Usman et al., (1987 J. Am. Chem. Soc., 109, 7845), Scaringe et al., (1990 Nucleic Acids Res., 18, 5433) and Wincott et al., supra, and made use of common nucleic acid protecting and coupling groups, such as dimethoxytrityl at the 5′-end, and phosphoramidites at the 3′-end. The average stepwise coupling yields were typically >98%.

Enzymatic nucleic acid molecules and antisense constructs are also synthesized from DNA templates using bacteriophage T7 RNA polymerase (Milligan and Uhlenbeck, 1989, Methods Enzymol. 180, 51). Enzymatic nucleic acid molecules and antisense constructs are purified by gel electrophoresis using general methods or are purified by high pressure liquid chromatography (HPLC; See Wincott et al., supra; the totality of which is hereby incorporated herein by reference) and are resuspended in water. The sequences of the chemically synthesized enzymatic nucleic acid molecules used in this study are shown below in Table III-VII. The sequences of the chemically synthesized antisense constructs used in this study are complimentary sequences to the Substrate sequences shown below as in Table III-VII.

### Example 4

### Enzymatic Nucleic Acid Molecule Cleavage of NOGO and NOGO Receptor RNA Target in vitro

Enzymatic nucleic acid molecules targeted to the human NOGO RNA are designed and synthesized as described above. These enzymatic nucleic acid molecules can be tested for cleavage activity in vitro, for example, using the following procedure. The target sequences and the nucleotide location within the NOGO receptor RNA are given in Tables III-VII.

Cleavage Reactions: Full-length or partially full-length, internally-labeled target RNA for enzymatic nucleic acid molecule cleavage assay is prepared by in vitro transcription in the presence of [a-³²P] CTP, passed over a G 50 Sephadex column by spin chromatography and used as substrate RNA without further purification. Alternately, substrates are 5′-³²P-end labeled using T4 polynucleotide kinase enzyme. Assays are performed by pre-warming a 2× concentration of purified enzymatic nucleic acid molecule in enzymatic nucleic acid molecule cleavage buffer (50 mM Tris-HCl, pH 7.5 at 37° C. 10 mM MgCl₂) and the cleavage reaction was initiated by adding the 2× enzymatic nucleic acid molecule mix to an equal volume of substrate RNA (maximum of 1-5 nM) that was also pre-warmed in cleavage buffer. As an initial screen, assays are carried out for 1 hour at 37° C. using a final concentration of either 40 nM or 1 mM enzymatic nucleic acid molecule, i.e., enzymatic nucleic acid molecule excess. The reaction is quenched by the addition of an equal volume of 95% formamide, 20 mM EDTA, 0.05% bromophenol blue and 0.05% xylene cyanol after which the sample is heated to 95° C. for 2 minutes, quick chilled and loaded onto a denaturing polyacrylamide gel. Substrate RNA and the specific RNA cleavage products generated by enzymatic nucleic acid molecule cleavage are visualized on an autoradiograph of the gel. The percentage of cleavage is determined by Phosphor Imager® quantitation of bands representing the intact substrate and the cleavage products.

### Example 5

### Nucleic Acid Inhibition of NOGO and NOGO Receptor Target RNA in vivo

Nucleic acid molecules targeted to the human NOGO and NOGO receptor RNA are designed and synthesized as described above. These nucleic acid molecules can be tested for cleavage activity in vivo, for example using the procedures described below. The target sequences and the nucleotide location within the NOGO receptor RNA are given in Tables III-VII.

Cell Culture

Spillmann et al., 1998, J. Biol. Chem., 273, 19283-19293, describe the purification and biochemical characterization of a high molecular mass protein of bovine spinal cord myelin (bNI-220) which exerts potent inhibition of neurite outgrowth of NGF-primed PC12 cells and chick DRG cells. This protein can be used to inhibit spreading of 3T3 fibroblasts and to induce collapse of chick DRG growth cones. The monoclonal antibody, mAb IN-1, can be used to fully neutralize the inhibitory activity of bNI-220, which is a presumed NOGO gene product. As such, nucleic acid molecules of the instant invention directed at the inhibition of NOGO expression can be used in place of mAb IN-1 in studying the inhibition of bNI-220 in cell culture experiments described in detail by Spillmann et al., supra. Criteria used in these experiments include the evaluation of spreading behavior of 3T3 fibroblasts, the neurite outgrowth response of PC12 cells, and the growth cone motility of chick DRG growth cones. Similarly, nucleic acid molecules of the instant invention that target NOGO or NOGO receptors can be used to evaluate inhibition of NOGO mediated activity in these cell types using the criteria described above.

Fournier et al., 2001, Nature, 409, 341 describe a mouse clone of the NOGO-66 receptor which is expressed in non-neuronal COS-7 cells. The transfected COS-7 cell line expresses NOGO-66 receptor protein on the cell surface. An antiserum developed to the NOGO-66 receptor can be used to specifically stain NOGO-66 receptor expressing cells by immunohistochemical staining. As such, an assay for screening nucleic acid-based inhibitors of NOGO-66 receptor expression is provided.

Animal Models

Bregman et al., 1995, Nature, 378, 498-501 and Z'Graggen et al., 1998, J. Neuroscience, 18, 4744, describe a rat based system for evaluating the role of myelin-associated neurite growth inhibitory proteins in vivo. Young adult Lewis rats receive a mid-thoracic microsurgical spinal cord lesion or a unilateral pyramidotomy. These animals are then treated with mAb IN-1 secreting hybridoma cell explants. A control population receive hybridoma explants which secrete horsreradish peroxidase (HRP) antibodies. Cyclosporin is used during the treatment period to allow hybridoma survival. Additional control rats receive either the spinal cord lesion without any further treatment or no lesion. After a 4-6 week recovery period, behavioral training is followed by the quantitative analysis of reflex and locomotor function. IN-1 treated animals demonstrate growth of corticospinal axons around the lesion site and into the spinal cord which persist past the longest time point of analysis (12 weeks). Furthermore, both reflex and locomotor function, including the functional recovery of fine motor control, is restored in IN-1 treated animals. As such, a robust animal model as described by Bregman et a.,l supra and Z'Graggen et al., supra, can be used to evaluate nucleic acid molecules of the instant invention when used in place of or in conjunction with mAb IN-1 toward use as modulators of neurite growth inhibitor function (eg. NOGO and NOGO receptor) in vivo.

Indications

The nucleic acids of the present invention can be used to treat a patient having a condition associated with the level of NOGO or NOGO receptor. One method of treatment comprises contacting cells of a patient with a nucleic acid molecule of the present invention, under conditions suitable for said treatment. Delivery methods and other methods of administration have been discussed herein and are commonly known in the art. Particular degenerative and disease states that can be associated with NOGO and NOGO receptor expression modulation include, but are not limited to, CNS injury, specifically spinal cord injury, cerebrovascular accident (CVA, stroke), Alzheimer's disease, dementia, multiple sclerosis (MS), chemotherapy-induced neuropathy, amyotrophic lateral sclerosis (ALS), Parkinson's disease, ataxia, Huntington's disease, Creutzfeldt-Jakob disease, muscular dystrophy, and/or other neurodegenerative disease states which respond to the modulation of NOGO and NOGO receptor expression.

The present body of knowledge in NOGO research indicates the need for methods to assay NOGO activity and for compounds that can regulate NOGO expression for research, diagnostic, and therapeutic use.

Other treatment methods comprise contacting cells of a patient with a nucleic acid molecule of the present invention and further comprise the use of one or more drug therapies under conditions suitable for said treatment. The use of monoclonal antibody (eg; mAb IN-1) treatment, growth factors, antiinflammatory compounds, for example methylprednisolone, calcium blockers, apoptosis inhibiting compounds, for example GM-1 ganglioside, and physical therapies, for example treadmill therapy, are all non-limiting examples of methods that can be combined with or used in conjunction with the nucleic acid molecules (e.g. ribozymes and antisense molecules) of the instant invention. Those skilled in the art will recognize that other drug compounds and therapies can be similarly be readily combined with the nucleic acid molecules of the instant invention (e.g. ribozymes and antisense molecules) are hence within the scope of the instant invention.

Diagnostic Uses

The nucleic acid molecules of this invention (e.g., enzymatic nucleic acid molecules) can be used as diagnostic tools to examine genetic drift and mutations within diseased cells or to detect the presence of NOGO and/or NOGO receptor RNA in a cell. The close relationship between enzymatic nucleic acid molecule activity and the structure of the target RNA allows the detection of mutations in any region of the molecule which alters the base-pairing and three-dimensional structure of the target RNA. By using multiple enzymatic nucleic acid molecules described in this invention, one can map nucleotide changes which are important to RNA structure and function in vitro, as well as in cells and tissues. Cleavage of target RNAs with enzymatic nucleic acid molecules can be used to inhibit gene expression and define the role (essentially) of specified gene products in the progression of disease. In this manner, other genetic targets can be defined as important mediators of the disease. These experiments can lead to better treatment of the disease progression by affording the possibility of combinational therapies (e.g., multiple enzymatic nucleic acid molecules targeted to different genes, enzymatic nucleic acid molecules coupled with known small molecule inhibitors, or intermittent treatment with combinations of enzymatic nucleic acid molecules and/or other chemical or biological molecules). Other in vitro uses of enzymatic nucleic acid molecules of this invention are well known in the art, and include detection of the presence of mRNAs associated with NOGO-related condition. Such RNA is detected by determining the presence of a cleavage product after treatment with a enzymatic nucleic acid molecule using standard methodology.

In a specific example, enzymatic nucleic acid molecules which cleave only wild-type or mutant forms of the target RNA are used for the assay. The first enzymatic nucleic acid molecule is used to identify wild-type RNA present in the sample and the second enzymatic nucleic acid molecule is used to identify mutant RNA in the sample. As reaction controls, synthetic substrates of both wild-type and mutant RNA are cleaved by both enzymatic nucleic acid molecules to demonstrate the relative enzymatic nucleic acid molecule efficiencies in the reactions and the absence of cleavage of the “non-targeted” RNA species. The cleavage products from the synthetic substrates also serve to generate size markers for the analysis of wild-type and mutant RNAs in the sample population. Thus each analysis requires two enzymatic nucleic acid molecules, two substrates and one unknown sample which is combined into six reactions. The presence of cleavage products is determined using an RNAse protection assay so that full-length and cleavage fragments of each RNA can be analyzed in one lane of a polyacrylamide gel. It is not absolutely required to quantify the results to gain insight into the expression of mutant RNAs and putative risk of the desired phenotypic changes in target cells. The expression of mRNA whose protein product is implicated in the development of the phenotype (i.e., NOGO) is adequate to establish risk. If probes of comparable specific activity are used for both transcripts, then a qualitative comparison of RNA levels will be adequate and will decrease the cost of the initial diagnosis. Higher mutant form to wild-type ratios are correlated with higher risk whether RNA levels are compared qualitatively or quantitatively. The use of enzymatic nucleic acid molecules in diagnostic applications contemplated by the instant invention is more fully described in George et al., U.S. Pat. Nos. 5,834,186 and 5,741,679, Shih et al., U.S. Pat. No. 5,589,332, Nathan et al., U.S. Pat. No 5,871,914, Nathan and Ellington, International PCT publication No. WO 00/24931, and Sullenger et al., International PCT publication No. WO 99/29842.

Additional Uses

Potential uses of sequence-specific enzymatic nucleic acid molecules of the instant invention can have many of the same applications for the study of RNA that DNA restriction endonucleases have for the study of DNA (Nathans et al., 1975 Ann. Rev. Biochem. 44:273). For example, the pattern of restriction fragments can be used to establish sequence relationships between two related RNAs, and large RNAs can be specifically cleaved to fragments of a size more useful for study. The ability to engineer sequence specificity of the enzymatic nucleic acid molecule is ideal for cleavage of RNAs of unknown sequence. Applicant has described the use of nucleic acid molecules to down-regulate gene expression of target genes in bacterial, microbial, fungal, viral, and eukaryotic systems including plant, or mammalian cells.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications can be made to the invention disclosed herein without departing from the scope and spirit of the invention. Thus, such additional embodiments are within the scope of the present invention and the following claims.

The invention illustratively described herein suitably can be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group. Other embodiments are within the claims that follow.

TABLE 1
Characteristics of naturally occurring ribozymes

Group I Introns
Size: ˜450 to >1000 nucleotides.
Requires a U in the target sequence immediately 5′ of the cleavage site.
Binds 4-6 nucleotides at the 5′-side of the cleavage site.
Reaction mechanism: attack by the 3′-OH of guanosine to generate
cleavage products with 3′-OH and 5′-guanosine.
Additional protein cofactors required in some cases to help folding and
maintenance of the active structure.
Over 300 known members of this class. Found as an intervening
sequence in Tetrahymena thermophila rRNA, fungal mitochondria,
choroplasts, phage T4, blue-green algae, and others.
Major structural features largely established through phylogenetic
comparisons, mutagenesis, and biochemical studies [i, ii].
Complete kinetic framework established for one ribozyme [iii, iv, v, vi].
Studies of ribozyme folding and substrate docking underway [vii , viii, ix].
Chemical modification investigation of important residues well established
[x, xi].
The small (4-6 nt) binding site may make this ribozyme too non-specific
for targeted RNA cleavage, however, the Tetrahymena group I intron has
been used
to repair a “defective” β-galactosidase message by the ligation of new β-
galactosidase sequences onto the defective message [xii].
RNAse P RNA (M1 RNA)
Size: ˜290 to 400 nucleotides.
RNA portion of a ubiquitous ribonucleoprotein enzyme.
Cleaves tRNA precursors to form mature tRNA [xiii].
Reaction mechanism: possible attack by M²⁺—OH to generate cleavage
products with 3′-OH and 5′-phosphate.
RNAse P is found throughout the prokaryotes and eukaryotes. The RNA
subunit has been sequenced from bacteria; yeast, rodents, and
primates.
Recruitment of endogenous RNAse P for therapeutic applications
is possible through hybridization of an External Guide Sequence
(EGS) to the target RNA [xiv, xv]
Important phosphate and 2′ OH contacts recently identified [xvi, xvii]
Group II Introns
Size: >1000 nucleotides.
Trans cleavage of target RNAs recently demonstrated [xviii, xix].
Sequence requirements not fully determined.
Reaction mechanism: 2′-OH of an internal adenosine generates
cleavage products with 3′-OH and a “lariat” RNA
containing a 3′-5′ and a 2′-5′ branch point.
Only natural ribozyme with demonstrated participation in DNA
cleavage [xx, xxi] in addition to RNA cleavage and
ligation.
Major structural features largely established through phylogenetic
comparisons [xxii]
Important 2′ OH contacts beginning to be identified [xxiii]
Kinetic framework under development [xxiv]
Neurospora VS RNA
Size: ˜444 nucleotides.
Trans cleavage of hairpin target RNAs recently demonstrated [xxv].
Sequence requirements not fully determined.
Reaction mechanism: attack by 2′-OH 5′ to the scissile bond
to generate cleavage products with 2′,3′-cyclic phosphate
and 5′-OH ends.
Binding sites and structural requirements not fully determined.
Only 1 known member of this class. Found in Neurospora VS RNA.
Hammerhead Ribozyme
(see text for references)
Size: ˜43 to 40 nucleotides.
Requires the target sequence UH immediately 5′ of the cleavage site.
Binds a variable number nucleotides on both sides of the cleavage site.
Reaction mechanism: attack by 2′-OH 5′ to the scissile
bond to generate cleavage
products with 2′,3′-cyclic phosphate and 5′-OH ends.
14 known members of this class. Found in a number of plant pathogens
(virusoids) that use RNA as the infectious agent.
Essential structural features largely defined, including 2 crystal
structures [xxvi, xxvii]
Minimal ligation activity demonstrated (for engineering through in
vitro selection) [xxviii]
Complete kinetic framework established for two or more ribozymes [xxix].
Chemical modification investigation of important residues well
established [xxx].
Hairpin Ribozyme
Size: ˜50 nucleotides.
Requires the target sequence GUC immediately 3′ of the cleavage site.
Binds 4-6 nucleotides at the 5′-side of the cleavage site and a
variable number to the 3′-side of the cleavage site.
Reaction mechanism: attack by 2′-OH 5′ to the scissile bond
to generate cleavage products with 2′,3′-cyclic phosphate
and 5′-OH ends.
3 known members of this class. Found in three plant pathogen
(satellite RNAs of the tobacco ringspot virus, arabis mosaic
virus and chicory yellow mottle virus)
which uses RNA as the infectious agent.
Essential structural features largely defined [xxxi, xxxii, xxxiii, xxxiv]
Ligation activity (in addition to cleavage activity) makes ribozyme
amenable to engineering through in vitro selection [xxxv]
Complete kinetic framework established for one ribozyme [xxxvi].
Chemical modification investigation of important residues
begun [xxxvii, xxxviii].
Hepatitis Delta Virus (HDV) Ribozyme
Size: ˜60 nucleotides.
Trans cleavage of target RNAs demonstrated [xxxix].
Binding sites and structural requirements not fully determined, although no
sequences 5′ of cleavage site are required. Folded ribozyme contains
a pseudoknot structure [xl]
Reaction mechanism: attack by 2′-OH 5′ to the scissile bond to
generate cleavage products with 2′,3′-cyclic phosphate and
5′-OH ends.
Only 2 known members of this class. Found in human HDV.
Circular form of HDV is active and shows increased nuclease
stability [Xli]

TABLE II

| | | | | | |
|---|---|---|---|---|---|
| Reagent | Equivalents | Amount | Wait Time* DNA | Wait Time* 2′-O-methyl | Wait Time* RNA |

A. 2.5 μmol Synthesis Cycle ABI 394 Instrument

| | | | | | |
|---|---|---|---|---|---|
| Phosphoramidites | 6.5 | 163 μL | 45 sec | 2.5 min | 7.5 min |
| S-Ethyl Tetrazole | 23.8 | 238 μL | 45 sec | 2.5 min | 7.5 min |
| Acetic Anhydride | 100 | 233 μL | 5 sec | 5 sec | 5 sec |
| N-Methyl | 186 | 233 μL | 5 sec | 5 sec | 5 sec |
| Imidazole | | | | | |
| TCA | 176 | 2.3 mL | 21 sec | 21 sec | 21 sec |
| Iodine | 11.2 | 1.7 mL | 45 sec | 45 sec | 45 sec |
| Beaucage | 12.9 | 645 μL | 100 sec | 300 sec | 300 sec |
| Acetonitrile | NA | 6.67 mL | NA | NA | NA |

B. 0.2 μmol Synthesis Cycle ABI 394 Instrument

| | | | | | |
|---|---|---|---|---|---|
| Phosphoramidites | 15 | 31 μL | 45 sec | 233 sec | 465 sec |
| S-Ethyl Tetrazole | 38.7 | 31 μL | 45 sec | 233 min | 465 sec |
| Acetic Anhydride | 655 | 124 μL | 5 sec | 5 sec | 5 sec |
| N-Methyl | 1245 | 124 μL | 5 sec | 5 sec | 5 sec |
| Imidazole | | | | | |
| TCA | 700 | 732 μL | 10 sec | 10 sec | 10 sec |
| Iodine | 20.6 | 244 μL | 15 sec | 15 sec | 15 sec |
| Beaucage | 7.7 | 232 μL | 100 sec | 300 sec | 300 sec |
| Acetonitrile | NA | 2.64 mL | NA | NA | NA |

| | | | | | |
|---|---|---|---|---|---|
| | | Amount: | | | |
| | Equivalents:DNA/ | DNA/2'-O- | | Wait Time* | Wait Time* |
| Reagent | 2'O-methyl/Ribo | methyl/Ribo | Wait Time* DNA | 2'-O-methyl | Ribo |

C. 0.2 μmol Synthesis Cycle 96 well Instrument

| | | | | | |
|---|---|---|---|---|---|
| Phosphoramidites | 22/33/66 | 40/60/120 μL | 60 sec | 180 sec | 360 sec |
| S-Ethyl Tetrazole | 70/105/210 | 40/60/120 μL | 60 sec | 180 min | 360 sec |
| Acetic Anhydride | 265/265/265 | 50/50/50 μL | 10 sec | 10 sec | 10 sec |
| N-Methyl | 502/502/502 | 50/50/50 μL | 10 sec | 10 sec | 10 sec. |
| Imidazole | | | | | |
| TCA | 238/475/475 | 250/500/500 μL | 15 sec | 15 sec | 15 sec |
| Iodine | 6.8/6.8/6.8 | 80/80/80 μL | 30 sec | 30 sec | 30 sec |
| Beaucage | 34/51/51 | 80/120/120 | 100 sec | 200 sec | 200 sec |
| Acetonitrile | NA | 1150/1150/1150 μL | NA | NA | NA |

*Wait time does not include contact time during delivery.

TABLE III
Human NOGO Receptor Hammerhead Ribozyme and Substrate
Sequence

| | | | | Rz |
|---|---|---|---|---|
| | | Seq | | Seq |
| Pos | Substrate | ID | Ribozyme | ID |
| 10 | CAACCCCU A CGAUGAAG | 1 | CUUCAUCG CUGAUGAGGCCGUUAGGCCGAA AGGGGUUG | 1024 |
| 26 | GAGGGCGU C CGCUGGAG | 2 | CUCCAGCG CUGAUGAGGCCGUUAGGCCGAA ACGCCCUC | 1025 |
| 108 | GCCUGCGU A UGCUACAA | 3 | UUGUAGCA CUGAUGAGGCCGUUAGGCCGAA ACGCAGGC | 1026 |
| 113 | CGUAUGCU A CAAUGAGC | 4 | GCUCAUUG CUGAUGAGGCCGUUAGGCCGAA AGCAUACG | 1027 |
| 177 | GUGGGCAU C CCUGCUGC | 5 | GCAGCAGG CUGAUGAGGCCGUUAGGCCGAA AUGCCCAC | 1028 |
| 198 | CACCGCAU C UUCCUGCA | 6 | UCCAGGAA CUGAUGAGGCCGUUAGCCCGAA AUGCGCUG | 1029 |
| 200 | GCGCAUCU U CCUGCACG | 7 | CGUGCAGG CUGAUGAGGCCGUUAGGCCGAA AGAUCCGC | 1030 |
| 201 | CGCAUCUU C CUGCACGG | 8 | CCGUGCAG CUGAUGAGGCCGUUAGGCCGAA AAGAUGCG | 1031 |
| 219 | AACCGCAU C UCGCAUGU | 9 | ACAUGCGA CUGAUGAGGCCGUUAGGCCGAA AUGCGGUU | 1032 |
| 221 | CCGCAUCU C GCAUGUGC | 10 | GCACAUGC CUGAUGAGGCCGUUACGCCCAA AGAUGCGG | 1033 |
| 242 | UCCCAGCU U CCGUGCCU | 11 | AGGCACGG CUGAUGAGGCCGUUAGGCCGAA AGCUGGCA | 1034 |
| 243 | GCCAGCUU C CGUGCCUG | 12 | CAGGCACG CUGAUGAGGCCGUUAGGCCCAA AAGCUGGC | 1035 |
| 261 | CGCAACCU C ACCAUCCU | 13 | AGGAUGGU CUGAUGAGGCCGUUAGGCCGAA AGGUUGCG | 1036 |
| 267 | CUCACCAU C CUCUCGCU | 14 | AGCCACAG CUGAUGAGGCCGUUAGGCCGAA AUGGUGAG | 1037 |
| 281 | GCUGCACU C GAAUGUGC | 15 | GCACAUUC CUGAUGAGGCCGUUAGGCCGAA AGUGCAGC | 1038 |
| 300 | GCCCGAAU U GAUGCGGC | 16 | GCCGCAUC CUGAUGAGGCCGUUAGGCCGAA AUUCGGGC | 1039 |
| 314 | GGCUGCCU U CACUGGCC | 17 | GGCCAGUG CUGAUGAGGCCGUUAGGCCGAA AGGCAGCC | 1040 |
| 315 | GCUGCCUU C ACUGGCCU | 18 | AGGCCAGU CUGAUGAGGCCGUUAGGCCGAA AAGGCAGC | 1041 |
| 330 | CUGGCCCU C CUGGACCA | 19 | UGCUCCAG CUGAUGAGGCCGUUAGGCCGAA AGGGCCAG | 1042 |
| 348 | CUGGACCU C AGCGAUAA | 20 | UUAUCGCU CUGAUGAGGCCGUUAGGCCGAA AGGUCCAG | 1043 |
| 355 | UCAGCGAU A AUGCACAG | 21 | CUGUGCAU CUGAUGAGGCCGUUAGGCCGAA AUCGCUGA | 1044 |
| 366 | GCACAGCU C CGGUCUGU | 22 | ACAGACCG CUGAUGAGGCCGUUAGGCCGAA AGCUGUGC | 1045 |
| 371 | GCUCCGGU C UGUGGACC | 23 | GOUCCACA CUGAUGAGGCCGUUAGGCCGAA ACCGGAGC | 1046 |
| 389 | UGCCACAU U CCACGGCC | 24 | GGCCGUGG CUGAUGAGGCCGUUAGGCCGAA AUGUGGCA | 1047 |
| 390 | GCCACAUU C CACGGCCU | 25 | AGGCCGUG CUGAUGAGGCCGUUAGGCCGAA AAUGUGGC | 1048 |
| 408 | GGCCGCCU A CACACGCU | 26 | AGCGUGUG CUGAUGAGGCCGUUAGGCCGAA AGGCGGCC | 1049 |
| 461 | GGGGCUGU U CCGCGGCC | 27 | GGCCGCGG CUGAUGAGGCCGUUAGGCCGAA ACAGCCCC | 1050 |
| 462 | GGGCUGUU C CGCGGCCU | 28 | AGGCCGCG CUGAUGAGGCCGUUAGGCCGAA AACAGCCC | 1051 |
| 485 | CCUGCAGU A CCUCUACC | 29 | GGUAGAGG CUGAUGAGGCCGUUAGGCCGAA ACUGCAGG | 1052 |
| 489 | CAGUACCU C UACCUGCA | 30 | UGCAGGUA CUGAUGAGGCCGUUAGGCCGAA AGGUACUG | 1053 |
| 491 | GUACCUCU A CCUGCAGG | 31 | CCUGCAGG CUGAUGAGGCCGUUAGGCCGAA AGAGGUAC | 1054 |
| 533 | UGACACCU U CCGCGACC | 32 | GGUCGCGG CUGAUGAGGCCGUUAGGCCGAA AGGUGUCA | 1055 |
| 534 | GACACCUU C CGCGACCU | 33 | AGGUCGCG CUGAUGAGGCCGUUAGGCCGAA AAGGUGUC | 1056 |
| 552 | GGCAACCU C ACACACCU | 34 | AGGUGUGU CUGAUGAGGCCGUUAGGCCGAA AGGUUGCC | 1057 |
| 561 | ACACACCU C UUCCUGCA | 35 | UGCAGGAA CUGAUGAGGCCGUUAGGCCGAA AGGUGUGU | 1058 |
| 563 | ACACCUCU U CCUGCACG | 36 | CGUGCAGG CUGAUGAGGCCGUUAGGCCGAA AGAGGUGU | 1059 |
| 564 | CACCUCUU C CUGCACGG | 37 | CCGUGCAG CUGAUGAGGCCGUUAGGCCGAA AAGAGGUG | 1060 |
| 582 | AACCGCAU C UCCAGCGU | 38 | ACGCUGGA CUGAUGAGGCCGUUAGGCCGAA AUGCGGUU | 1061 |
| 584 | CCGCAUCU C CAGCGUGC | 39 | GCACGCUG CUGAUGAGGCCGUUAGGCCGAA AGAUGCGG | 1062 |
| 605 | GCGCGCCU U CCGUGGGC | 40 | GCCCACGG CUGAUGAGGCCGUUAGGCCGAA AGOCOCOC | 1063 |
| 606 | CGCGCCUU C CGUGGGCU | 41 | AGCCCACG CUGAUGAGGCCGUUAGGCCGAA AAGGCGCG | 1064 |
| 624 | CACAGCCU C GACCGUCU | 42 | AGACGGUC CUGAUGAGGCCGUUAGGCCGAA AGGCUGUG | 1065 |
| 631 | UCGACCGU C UCCUACUG | 43 | CAGUAGGA CUGAUGAGGCCGUUAGGCCGAA ACGGUCGA | 1066 |
| 633 | GACCGUCU C CUACUGCA | 44 | UGCAGUAG CUGAUGAGGCCGUUAGGCCGAA AGACGGUC | 1067 |
| 636 | CGUCUCCU A CUGCACCA | 45 | UGGUGCAC CUGAUGAGGCCGUUAGGCCGAA AGGAGACG | 1068 |
| 677 | GCAUGCCU U CCGUGACC | 46 | GGUCACGG CUCAUGAGGCCGUUAGGCCGAA AGGCAUGC | 1069 |
| 678 | CAUGCCUU C CGUGACCU | 47 | AGGUCACG CUCAUGAGGCCGUUAGGCCGAA AAGGCAUG | 1070 |
| 687 | CGUGACCU U GGCCGCCU | 48 | AGGCGGCC CUGAUGAGGCCGUUAGGCCGAA AGGUCACG | 1071 |
| 696 | GGCCGCCU C AUGACACU | 49 | AGUGUCAU CUGAUGAGGCCGUUAGGCCGAA AGGCGGCC | 1072 |
| 705 | AUGACACU C UAUCUGUU | 50 | AACAGAUA CUGAUGAGGCCGUUAGGCCGAA AGUGUCAU | 1073 |
| 707 | GACACUCU A UCUGUUUG | 51 | CAAACAGA CUGAUGAGGCCGUUAGGCCGAA AGAGUGUC | 1074 |
| 709 | CACUCUAU C UGUUUGCC | 52 | GGCAAACA CUGAUGAGGCCGUUAGGCCGAA AUAGAGUG | 1075 |
| 713 | CUAUCUGU U UGCCAACA | 53 | UGUUGGCA CUGAUGAGGCCGUUAGGCCGAA ACAGAUAG | 1076 |
| 714 | UAUCUGUU U GCCAACAA | 54 | UUGUUGGC CUGAUGAGGCCGUUAGGCCGAA AACAGAUA | 1077 |
| 724 | CCAACAAU C UAUCAGCG | 55 | CGCUGAUA CUGAUGAGGCCGUUAGGCCGAA AUUGUUGG | 1078 |
| 726 | AACAAUCU A UCAGCGCU | 56 | AGCGCUGA CUGAUGAGGCCGUUAGGCCGAA AGAUUGUU | 1079 |
| 728 | CAAUCUAU C AGCGCUGC | 57 | GCAGCGCU CUGAUGAGGCCGUUAGGCCGAA AUAGAUUG | 1080 |
| 773 | CCUGCAGU A CCUGAGGC | 58 | GCCUCAGG CUGAUGAGGCCGUUAGGCCGAA ACUGCAGG | 1081 |
| 783 | CUGAGGCU C AACGACAA | 59 | UUGUCGUU CUGAUGAGGCCGUUAGGCCGAA AGOCUCAG | 1082 |
| 825 | CGCCCACU C UGGGCCUG | 60 | CAGGCCCA CUGAUGAGGCCGUUAGGCCGAA AGUGGGCG | 1083 |
| 845 | GCAGAAGU U CCGCGGCU | 61 | AGCCGCGG CUGAUGAGGCCGUUAGGCCGAA ACUUCUGC | 1084 |
| 846 | CAGAAGUU C CGCGGCUC | 62 | GAGCCGCG CUGAUGAGGCCGUUAGGCCGAA AACUUCUG | 1085 |
| 854 | CCGCGGCU C CUCCUCCG | 63 | CGGAGGAG CUGAUGAGGCCGUUAGGCCGAA AGCCGCGG | 1086 |
| 857 | CGGCUCCU C CUCCGAGG | 64 | CCUCGGAG CUGAUGAGGCCGUUAGGCCGAA AGGAGCCG | 1087 |
| 860 | CUCCUCCU C CGAGGUGC | 65 | GCACCUCG CUGAUGAGGCCGUUAGGCCGAA AGGAGGAG | 1088 |
| 879 | UGCAGCCU C CCGCAACG | 66 | CGUUGCGG CUGAUGAGGCCGUUAGGCCGAA AGGCUGCA | 1089 |
| 906 | CGUGACCU C AAACGCCU | 67 | AGGCGUUU CUGAUGAGGCCGUUAGGCCGAA AGGUCACG | 1090 |
| 915 | AAACGCCU A GCUGCCAA | 68 | UUGGCAGC CUGAUGAGGCCGUUAGGCCGAA AGGCGUUU | 1091 |
| 958 | CCGGCCCU U ACCAUCCC | 69 | GGGAUGGU CUGAUGAGGCCGUUAGGCCGAA AGGGCCGG | 1092 |
| 959 | CGGCCCUU A CCAUCCCA | 70 | UGGGAUGG CUGAUGAGGCCGUUAGGCCGAA AAGGGCCG | 1093 |
| 964 | CUUACCAU C CCAUCUGG | 71 | CCAGAUGG CUGAUGAGGCCGUUAGGCCGAA AUGGUAAG | 1094 |
| 969 | CAUCCCAU C UGGACCGG | 72 | CCGGUCCA CUGAUGAGGCCGUUAGGCCGAA AUGGGAUG | 1095 |
| 1008 | CUGGGGCU U CCCAAGUG | 73 | CACUUGGG CUGAUGAGGCCGUUAGGCCGAA AGCCCCAG | 1096 |
| 1009 | UGGGGCUU C CCAAGUGC | 74 | GCACUUGG CUGAUGAGGCCGUUAGGCCGAA AAGCCCCA | 1097 |
| 1046 | CAAGGCCU C AGUACUGG | 75 | CCAGUACU CUGAUGAGGCCGUUAGGCCGAA AGGCCUUG | 1098 |
| 1050 | GCCUCAGU A CUGGAGCC | 76 | GGCUCCAG CUGAUGAGGCCGUUAGGCCGAA ACUGAGGC | 1099 |
| 1072 | GACCAGCU U CGGCAGGC | 77 | GCCUGCCG CUGAUGAGGCCGUUAGGCCGAA ACCUGGUC | 1100 |
| 1073 | ACCAGCUU C GGCAGGCA | 78 | UGCCUGCC CUGAUGAGGCCGUUAGGCCGAA AAGCUGGU | 1101 |
| 1133 | CAACGGCU C UGGCCCAC | 79 | GUGGGCCA CUGAUGAGGCCGUUAGGCCGAA AGCCGUUG | 1102 |
| 1149 | CGGCACAU C AAUGACUC | 80 | GAGUCAUU CUGAUGAGGCCGUUAGGCCGAA AUGUGCCG | 1103 |
| 1157 | CAAUGACU C ACCCUUUG | 81 | CAAAGGGU CUGAUGAGGCCGUUAGGCCGAA AGUCAUUG | 1104 |
| 1163 | CUCACCCU U UGGGACUC | 82 | GAGUCCCA CUGAUGAGGCCGUUAGGCCGAA AGGGUGAG | 1105 |
| 1164 | UCACCCUU U GGGACUCU | 83 | AGAGUCCC CUGAUGAGGCCGUUAGGCCGAA AAGGGUGA | 1106 |
| 1171 | UUGGGACU C UGCCUGGC | 84 | GCCAGGCA CUGAUGAGGCCGUUAGGCCGAA AGUCCCAA | 1107 |
| 1181 | GCCUGGCU C UGCUGAGC | 85 | GCUCAGCA CUGAUGAGGCCGUUAGGCCGAA AGCCAGGC | 1108 |
| 1197 | CCCCCGCU C ACUGCAGU | 86 | ACUGCAGU CUGAUGAGGCCGUUAGGCCGAA AGCGGGGG | 1109 |
| 1220 | CGAGGGCU C CGAGCCAC | 87 | GUGGCUCG CUGAUGAGGCCGUUAGGCCGAA AGCCCUCG | 1110 |
| 1235 | ACCAGGGU U CCCCACCU | 88 | AGGUGGGG CUGAUGAGGCCGUUAGGCCGAA ACCCUGGU | 1111 |
| 1238 | CCAGGGUU C CCCACCUC | 89 | GAGGUGGG CUGAUGAGGCCGUUAGGCCGAA AACCCUGG | 1112 |
| 1244 | CCCCACCU C GGGCCCUC | 90 | GAGGGCCC CUGAUGAGGCCGUUAGGCCGAA AGGUGGGG | 1113 |
| 1252 | CGGGCCCU C GCCGGAGG | 91 | CCUCCGGC CUGAUGAGGCCGUUAGGCCGAA AGGGCCCG | 1114 |
| 1270 | CAGGCUGU U CACGCAAG | 92 | CUUGCGUG CUGAUGAGGCCGUUAGGCCGAA ACAGCCUG | 1115 |
| 1271 | AGGCUGUU C ACGCAAGA | 93 | UCUUGCGU CUGAUGAGGCCGUUAGGCCGAA AACAGCCU | 1116 |
| 1303 | ACUGCCGU C UGGGCCAG | 94 | CUGGCCCA CUGAUGAGGCCGUUAGGCCGAA ACGGCAGU | 1117 |
| 1343 | UGGUGACU C AGAAGGCU | 95 | AGCCUUCU CUGAUGAGGCCGUUAGGCCGAA AGUCACCA | 1118 |
| 1352 | AGAAGGCU C AGGUGCCC | 96 | GGGCACCU CUGAUGAGGCCGUUAGGCCGAA AGCCUUCU | 1119 |
| 1362 | GGUGCCCU A CCCAGCCU | 97 | AGGCUGGG CUGAUGAGGCCGUUAGGCCGAA AGGGCACC | 1120 |
| 1371 | CCCAGCCU C ACCUGCAG | 98 | CUGCAGGU CUGAUGAGGCCGUUAGGCCGAA AGOCUGGG | 1121 |
| 1383 | UGCAGCCU C ACCCCCCU | 99 | AGGGGGGU CUGAUGAGGCCGUUAGGCCGAA AGGCUGCA | 1122 |
| 1422 | ACAGUGCU U GGGCCCUG | 100 | CAGGGCCC CUGAUGAGGCCGUUAGGCCGAA AGCACUGU | 1123 |

Input Sequence = AB020693. Cut Site = CH/.
Arm Length = 8. Core Sequence = CUGAUGAG GCCGUUAGGC CGAA
AB020693 (Homo sapiens mRNA for K1AA0886 protein (Nogo-A); 4053 bp)
Underlined region can be any X sequence or linker, as described herein.

Table IV
Human NOGO Receptor NCH Ribozyme and Substrate Seqeunce

| | | | | Rz |
|---|---|---|---|---|
| | | Seq | | Seq |
| Pos | Substrate | ID | Ribozyme | ID |
| 9 | CCAACCCC U ACGAUGAA | 101 | UUCAUCGU CUGAUGAGGCCGUUAGGCCGAA IGGGUUGG | 1124 |
| 27 | AGGGCGUC C GCUGGAGG | 102 | CCUCCAGC CUGAUGAGGCCGUUAGGCCGAA IACGCCCU | 1125 |
| 30 | GCGUCCGC U GGAGGGAG | 103 | CUCCCUCC CUGAUGAGGCCGUUAGGCCGAA ICGGACGC | 1126 |
| 40 | GAGGGAGC C GGCUGCUG | 104 | CAGCAGCC CUGAUGAGGCCGUUAGGCCGAA ICUCCCUC | 1127 |
| 44 | GAGCCGGC U GCUGGCAU | 105 | AUGCCAGC CUGAUGAGGCCGUUAGGCCGAA ICCGGCUC | 1128 |
| 47 | CCGGCUCC U GGCAUGGG | 106 | CCCAUGCC CUGAUGAGGCCGUUAGGCCGAA ICAGCCGG | 1129 |
| 51 | CUUCUGGC A UGGGUGCU | 107 | ACCACCCA CUGAUGAGGCCGUUAGGCCGAA ICCAGCAG | 1130 |
| 59 | AUGGGUGC U CUCGCUGC | 108 | GCAGCCAC CUGAUGAGGCCGUUAGGCCGAA ICACCCAU | 1131 |
| 65 | UCUGUGUC U GCAGGCCU | 109 | AGGCCUGC CUGAUGAGGCCGUUAGGCCGAA ICCACAGC | 1132 |
| 68 | GUGGCUGC A GGCCUGGC | 110 | GCCAGGCC CUGAUGAGGCCGUUAGGCCGAA ICAGCCAC | 1133 |
| 72 | CUGCAGGC C UGGCAGGU | 111 | ACCUGCCA CUGAUGAGGCCGUUAGGCCGAA ICCUGCAG | 1134 |
| 73 | UGCAGGCC U GGCAGGUG | 112 | CACCUGCC CUGAUGAGGCCGUUAGGCCGAA IGCCUGCA | 1135 |
| 77 | GGCCUGGC A GGUGGCAG | 113 | CUGCCACC CUGAUGAGGCCGUUAGGCCGAA ICCAGGCC | 1136 |
| 84 | CAGGUGGC A GCCCCAUG | 114 | CAUGGGGC CUGAUGAGGCCGUUAGGCCGAA ICCACCUG | 1137 |
| 87 | GUGGCAGC C CCAUGCCC | 115 | GGGCAUGG CUGAUGAGGCCGUUAGGCCGAA ICUGCCAC | 1138 |
| 88 | UGGCAGCC C CAUGCCCA | 116 | UGGGCAUG CUGAUGAGGCCGUUAGGCCGAA IGCUGCCA | 1139 |
| 89 | GGCAGCCC C AUGCCCAG | 117 | CUGGGCAU CUGAUGAGGCCGUUAGGCCGAA IGGCUGCC | 1140 |
| 90 | GCAGCCCC A UGCCCAGG | 118 | CCUGGGCA CUGAUGAGGCCGUUAGGCCGAA IGGGCUGC | 1141 |
| 94 | CCCCAUGC C CAGGUGCC | 119 | GGCACCUG CUGAUGAGGCCGUUAGGCCGAA ICAUGGGG | 1142 |
| 95 | CCCAUGCC C AGGUGCCU | 120 | AGGCACCU CUGAUGAGGCCGUUAGGCCGAA IGCAUGGG | 1143 |
| 96 | CCAUGCCC A GGUGCCUG | 121 | CAGGCACC CUGAUGAGGCCGUUAGGCCGAA IGGCAUGG | 1144 |
| 102 | CCAGGUGC C UGCGUAUG | 122 | CAUACGCA CUGAUGAGGCCGUUAGGCCGAA ICACCUGO | 1145 |
| 103 | CAGGUGUC U GCGUAUGC | 123 | GCAUACGC CUGAUCAGGCCGUUAGGCCGAA IGCACCUG | 1146 |
| 112 | GCGUAUGC U ACAAUGAG | 124 | CUCAUUGU CUGAUGAGGCCGUUAGGCCGAA ICAUACGC | 1147 |
| 115 | UAUGCUAC A AUGAGCCC | 125 | GGGCUCAU CUGAUGAGGCCGUUAGGCCGAA IUAGCAUA | 1148 |
| 122 | CAAUGAGC C CAAGGUGA | 126 | UCACCUUG CUGAUGAGGCCGUUAGGCCGAA ICUCAUUG | 1149 |
| 123 | AAUGAGCC C AAGGUGAC | 127 | GUCACCUU CUGAUGAGGCCGUUAGGCCGAA IGCUCAUU | 1150 |
| 124 | AUGAGCCC A AGGUGACG | 128 | CGUCACCU CUGAUGAGGCCGUUAGGCCGAA IGGCUCAU | 1151 |
| 135 | GUGACGAC A AGCUGCCC | 129 | GGGCAGCU CUGAUGAGGCCGUUAGGCCGAA IUCGUCAC | 1152 |
| 139 | CGACAAGC U GCCCCCAG | 130 | CUGGGGGC CUGAUGAGGCCGUUAGGCCGAA ICUUGUCG | 1153 |
| 142 | CAAGCUGC C CCCAGCAG | 131 | CUGCUGGG CUGAUGAGGCCGUUAGGCCGAA ICAGCUUG | 1154 |
| 143 | AAGCUGCC C CCAGCAGG | 132 | CCUGCUGG CUGAUGAGGCCGUUAGGCCGAA IGCAGCUU | 1155 |
| 144 | AGCUGCCC C CAGCAGGG | 133 | CCCUGCUG CUGAUGAGGCCGUUAGGCCGAA IGGCAGCU | 1156 |
| 145 | GCUGCCCC C AGCAGGGC | 134 | GCCCUGCU CUGAUGAGGCCGUUAGGCCGAA IGGGCAGC | 1157 |
| 146 | CUGCCCCC A GCAGGGCC | 135 | GGCCCUGC CUGAUGAGGCCGUUAGGCCGAA IGGGGCAG | 1158 |
| 149 | CCCCCAGC A GGGCCUGC | 136 | GCAGGCCC CUGAUGAGGCCGUUAGGCCGAA ICUGGGGG | 1159 |
| 154 | AGCAGGGC C UGCAGGCU | 137 | AGCCUGCA CUGAUGAGGCCGUUAGGCCGAA ICCCUGCU | 1160 |
| 155 | GCAGGGCC U GCAGGCUG | 138 | CAGCCUGC CUGAUGAGGCCGUUAGGCCGAA IGCCCUGC | 1161 |
| 158 | GGGCCUGC A GGCUGUGC | 139 | GCACAGCC CUGAUGAGGCCGUUAGGCCGAA ICAGGCCC | 1162 |
| 162 | CUGCAGGC U GUGCCCGU | 140 | ACGGGCAC CUGAUGAGGCCGUUAGGCCGAA ICCUGCAG | 1163 |
| 167 | GGCUGUGC C CGUGGGCA | 141 | UGCCCACG CUGAUGAGGCCGUUAGGCCGAA ICACAGCC | 1164 |
| 168 | GCUGUGCC C GUGGGCAU | 142 | AUGCCCAC CUGAUGAGGCCGUUAGGCCGAA IGCACAGC | 1165 |
| 175 | CCGUGGGC A UCCCUGCU | 143 | AGCAGGGA CUGAUGAGGCCGUUAGGCCGAA ICCCACGG | 1166 |
| 176 | UGGGCAUC C CUGCUGCC | 144 | GGCAGCAG CUGAUGAGGCCGUUAGGCCGAA IAUGCCCA | 1167 |
| 179 | GGGCAUCC C UGCUGCCA | 145 | UGGCAGCA CUGAUGAGGCCGUUAGGCCGAA IGAUGCCC | 1168 |
| 180 | GGCAUCCC U GCUGCCAG | 146 | CUGGCAGC CUGAUGAGGCCGUUAGGCCGAA IGGAUGCC | 1169 |
| 183 | AUCCCUGC U GCCAGCCA | 147 | UGGCUGGC CUGAUGAGGCCGUUAGGCCGAA ICAGGGAU | 1170 |
| 186 | CCUGCUGC C AGCCAGCG | 148 | CGCUGGCU CUGAUGAGGCCGUUAGGCCGAA ICAGCAGG | 1171 |
| 187 | CUGCUGCC A GCCAGCGC | 149 | OCUCUGOC CUGAUGAGGCCGUUAGGCCGAA IGCAGCAG | 1172 |
| 190 | CUGCCAGC C AGCGCAUC | 150 | GAUGCGCU CUGAUGAGGCCGUUAGGCCGAA ICUGUCAG | 1173 |
| 191 | UGCCAGCC A GCGCAUCU | 151 | AGAUGCGC CUGAUGAGGCCGUUAGGCCGAA IGCUGGCA | 1174 |
| 196 | GCCAGCGC A UCCUCCUG | 152 | CAGGAAGA CUGAUGAGGCCGUUAGGCCGAA ICGCUGGC | 1175 |
| 199 | AGCGCAUC U UCCUCCAC | 153 | GUGCAGGA CUGAUGAGGCCGUUAGGCCGAA IAUGCGCU | 1176 |
| 202 | GCAUCUUC C UGCACGGC | 154 | GCCGUGCA CUGAUGAGGCCGUUAGGCCCAA IAAGAUGC | 1177 |
| 203 | CAUCUUCC U GCACGGCA | 155 | UGCCGUGC CUGAUGAGGCCGUUAGGCCGAA IGAAGAUG | 1178 |
| 206 | CUUCCUGC A CGGCAACC | 156 | GGUUGCCG CUGAUGAGGCCGUUAGGCCGAA ICAGGAAG | 1179 |
| 569 | CUUCCUGC A CGGCAACC | 156 | GGUUGCCG CUGAUGAGGCCGUUAGGCCGAA ICAGGAAG | 1180 |
| 211 | UGCACGGC A ACCGCAUC | 157 | GAUGCGGU CUGAUGAGGCCGUUAGGCCGAA ICCGUGCA | 1181 |
| 574 | UGCACGGC A ACCOCAUC | 157 | GAUGCGGU CUGAUGAGGCCGUUAGGCCGAA ICCGUGCA | 1182 |
| 214 | ACGGCAAC C GCAUCUCG | 158 | CGAGAUGC CUGAUGAGGCCGUUAGGCCGAA IUUGCCGU | 1183 |
| 217 | GCAACCGC A UCUCGCAU | 159 | AUGCGAGA CUGAUGAGGCCGUUAGGCCGAA ICGGUUGC | 1184 |
| 220 | ACCGCAUC U CGCAUGUG | 160 | CACAUGCG CUGAUGAGGCCGUUAGGCCGAA IAUGCGGU | 1185 |
| 224 | CAUCUCUC A UGUGCCAG | 161 | CUGUCACA CUGAUGAGGCCGUUAGGCCGAA ICGAGAUG | 1186 |
| 230 | UCAUGUOC C AGCUGCCA | 162 | UGGCAGCU CUGAUGAGGCCGUUAGGCCGAA ICACAUGC | 1187 |
| 231 | CAUGUGCC A GCUGCCAG | 163 | CUGGCAGC CUGAUGAGGCCGUUAGGCCGAA IGCACAUG | 1188 |
| 234 | GUGCCAGC U GCCAGCUU | 164 | AAGCUGGC CUGAUGAGGCCGUUAGGCCGAA ICUGGCAC | 1139 |
| 237 | CCAGCUGC C AGCUUCCG | 165 | CGGAAGCU CUGAUGAGGCCGUUAGGCCGAA ICAGCUGG | 1190 |
| 238 | CAGCUGCC A GCUUCCGU | 166 | ACGGAAGC CUGAUGAGGCCGUUAGGCCGAA IGCAGCUG | 1191 |
| 241 | CUGCCAGC U UCCGUGCC | 167 | GGCACGGA CUGAUGAGGCCGUUAGGCCGAA ICUGGCAG | 1192 |
| 244 | CCAGCUUC C GUGCCUGC | 168 | GCAGGCAC CUGAUGAGGCCGUUAGGCCGAA IAAGCUGG | 1193 |
| 249 | UUCCGUGC C UGCCGCAA | 169 | UUGCGGCA CUGAUGAGGCCGUUAGGCCGAA ICACGGAA | 1194 |
| 250 | UCCGUGCC U GCCGCAAC | 170 | GUUGCGGC CUGAUGAGGCCGUUAGGCCGAA IGCACGGA | 1195 |
| 253 | GUGCCUGC C GCAACCUC | 171 | GAGGUUGC CUGAUGAGGCCGUUAGGCCGAA ICAGGCAC | 1196 |
| 256 | CCUGCCGC A ACCUCACC | 172 | GGUGAGGU CUGAUGAGGCCGUUAGGCCGAA ICGGCAGG | 1197 |
| 259 | GCCGCAAC C UCACCAUC | 173 | GAUGGUGA CUGAUGAGGCCGUUAGGCCGAA IUUGCGGC | 1198 |
| 260 | CCGCAACC U CACCAUCC | 174 | GGAUGGUG CUGAUGAGGCCGUUAGGCCGAA IGUUGCGG | 1199 |
| 262 | GCAACCUC A CCAUCCUG | 175 | CAGGAUGG CUGAUGAGGCCGUUAGGCCGAA IAGGUUGC | 1200 |
| 264 | AACCUCAC C AUCCUGUG | 176 | CACAGGAU CUGAUGAGGCCGUUAGGCCGAA IUGAGGUU | 1201 |
| 265 | ACCUCACC A UCCUGUGG | 177 | CCACAGGA CUGAUGAGGCCGUUAGGCCGAA IGUGAGGU | 1202 |
| 268 | UCACCAUC C UGUGGCUG | 178 | CAUCCACA CUGAUGAGGCCGUUAGGCCGAA IAUGGUGA | 1203 |
| 269 | CACCAUCC U GUGGCUGC | 179 | GCAGCCAC CUGAUGAGGCCGUUAGGCCGAA IGAUGGUG | 1204 |
| 275 | CCUGUGGC U GCACUCGA | 180 | UCGAGUGC CUGAUGAGGCCGUUAGGCCGAA ICCACAGG | 1205 |
| 278 | GUGGCUGC A CUCGAAUG | 181 | CAUUCGAG CUGAUGAGGCCGUUAGGCCGAA ICAGCCAC | 1206 |
| 280 | GGCUGCAC U CGAAUGUG | 182 | CACAUUCG CUGAUGAGGCCGUUAGGCCGAA IUGCAGCC | 1207 |
| 290 | GAAUGUGC U GGCCCGAA | 183 | UUCGGGCC CUGAUGAGGCCGUUAGGCCGAA ICACAUUC | 1208 |
| 294 | GUGCUGGC C CGAAUUGA | 184 | UCAAUUCG CUGAUGAGGCCGUUAGGCCGAA ICCAGCAC | 1209 |
| 295 | UGCUGGCC C GAAUUGAU | 185 | AUCAAUUC CUGAUCAGGCCGUUAGGCCGAA IGCCACCA | 1210 |
| 309 | GAUGCGGC U GCCUUCAC | 186 | GUGAAGGC CUGAUGAGGCCGUUAGGCCGAA ICCGCAUC | 1211 |
| 312 | GCGGCUGC C UUCACUGG | 187 | CCAGUGAA CUGAUGAGGCCGUUAGGCCGAA ICAGCCGC | 1212 |
| 313 | CGGCUGCC U UCACUGGC | 188 | GCCAGUGA CUGAUGAGGCCGUUAGGCCGAA IGCAGCCG | 1213 |
| 316 | CUGCCUUC A CUGGCCUG | 189 | CAGGCCAG CUGAUGAGGCCGUUAGGCCGAA IAAGGCAG | 1214 |
| 318 | UCCUUCAC U GGCCUGGC | 190 | GCCAGGCC CUGAUGAGGCCGUUAGGCCGAA IUGAAGGC | 1215 |
| 322 | UCACUGGC C UGGCCCUC | 191 | GAGGGCCA CUGAUGAGGCCGUUAGGCCGAA ICCAGUGA | 1216 |
| 323 | CACUGGCC U GGCCCUCC | 192 | GGAGGGCC CUGAUGAGGCCGUUAGGCCGAA IGCCAGUG | 1217 |
| 327 | GGCCUGGC C CUCCUGGA | 193 | UCCAGGAG CUGAUGAGGCCGUUAGGCCGAA ICCAGGOC | 1218 |
| 328 | GCCUGGCC C UCCUGGAG | 194 | CUCCAGGA CUGAUGAGGCCGUUAGGCCGAA IGCCAGGC | 1219 |
| 329 | CCUGGCCC U CCUGGAGC | 195 | GCUCCAGG CUGAUGAGGCCGUUAGGCCGAA IGGCCAGG | 1220 |
| 331 | UGGCCCUC C UGGAGCAG | 196 | CUCCUCCA CUGAUGAGGCCGUUAGGCCGAA IAGGGCCA | 1221 |
| 332 | GGCCCUCC U GGAGCAGC | 197 | GCUGCUCC CUGAUGAGGCCGUUAGGCCGAA IGAGGGCC | 1222 |
| 338 | CCUGGAGC A GCUCGACC | 198 | GGUCCAGC CUGAUGAGGCCGUUAGGCCGAA ICUCCAGG | 1223 |
| 341 | GGAGCAGC U GUACCUCA | 199 | UGAGGUCC CUGAUGAGGCCGUUAGGCCGAA ICUGCUCC | 1224 |
| 346 | ACCUGGAC C UCAGCGAU | 200 | AUCGCUGA CUGAUGAGGCCGUUAGGCCGAA IUCCAGCU | 1225 |
| 347 | GCUGGACC U CAGCGAUA | 201 | UAUCGCUG CUGAUGAGGCCGUUAGGCCGAA IGUCCAGC | 1226 |
| 349 | UGGACCUC A GCGAUAAU | 202 | AUUAUCGC CUGAUGAGGCCGUUAGGCCGAA IAGGUCCA | 1227 |
| 360 | GAUAAUGC A CAGCUCCG | 203 | CGGAGCUG CUGAUGAGGCCGUUAGGCCGAA ICAUUAUC | 1228 |
| 362 | UAAUGCAC A GCUCCGGU | 204 | ACCGGAGC CUGAUGAGGCCGUUAGGCCGAA IUGCAUUA | 1229 |
| 365 | UGCACAGC U COGGUCUG | 205 | CAGACCGG CUGAUGAGGCCGUUAGGCCGAA ICUGUGCA | 1230 |
| 367 | CACAUCUC C GGUCUGUG | 206 | CACAGACC CUGAUGAGGCCGUUAGGCCGAA IAGCUGUG | 1231 |
| 372 | CUCCOGUC U GUGGACCC | 207 | GGGUCCAC CUGAUGAGGCCGUUAGGCCGAA IACCGGAG | 1232 |
| 379 | CUGUGGAC C CUGCCACA | 208 | UGUGGCAG CUGAUGAGGCCGUUAGGCCGAA IUCCACAG | 1233 |
| 380 | UGUGGACC C UGCCACAU | 209 | AUGUGGCA CUGAUGAGGCCGUUAGGCCGAA IGUCCACA | 1234 |
| 381 | GUGGACCC U GCCACAUU | 210 | AAUGUGGC CUGAUGAGGCCGUUAGGCCGAA IGGUCCAC | 1235 |
| 384 | GACCCUGC C ACAUUCCA | 211 | UGGAAUGU CUGAUGAGGCCGUUAGGCCGAA ICAGUCUC | 1236 |
| 385 | ACCCUGCC A CAUUCCAC | 212 | GUGGAAUG CUGAUGAGGCCGUUAGGCCGAA IGCAGGGU | 1237 |
| 387 | UCUGOCAC A UUCCACGG | 213 | CCGUGGAA CUGAUGAGGCCGUUAGGCCGAA IUGGCAGG | 1238 |
| 391 | CCACAUUC C ACGGCCUG | 214 | CAGGCCGU CUGAUGAGGCCGUUAGGCCGAA IAAUGUGG | 1239 |
| 392 | CACAUUCC A CGGCCUGG | 215 | CCAGGCCG CUGAUGAGGCCGUUAGGCCGAA IGAAUGUG | 1240 |
| 397 | UCCACGGC C UGGGCCGC | 216 | GCGGCCCA CUGAUGAGGCCGUUAGGCCGAA ICCOUGGA | 1241 |
| 398 | CCACGGCC U GGGCCGCC | 217 | GGCGGCCC CUGAUGAGGCCGUUAGGCCGAA IGCCGUGG | 1242 |
| 403 | GCCUGGGC C GCCUACAC | 218 | GUGUAGGC CUGAUGAGGCCGUUAGGCCGAA ICCCAGGC | 1243 |
| 406 | UGGGCCGC C UACACACG | 219 | CGUGUGUA CUGAUGAGGCCGUUAGGCCGAA ICGGCCCA | 1244 |
| 407 | GGGCCGCC U ACACACGC | 220 | GCGUGUGU CUGAUGAGGCCGUUAGGCCGAA IGCGGCCC | 1245 |
| 410 | CCGCCUAC A CACGCUGC | 221 | GCAGCGUG CUGAUGAGGCCGUUAGGCCGAA IUAGGCGG | 1246 |
| 412 | GCCUACAC A CGCUGCAC | 222 | GUGCAGCG CUGAUGAGGCCGUUAGGCCGAA IUGUAGGC | 1247 |
| 416 | ACACACGC U GCACCUGG | 223 | CCAGGUGC CUGAUGAGGCCGUUAGGCCGAA ICGUGUGU | 1248 |
| 419 | CACGCUGC A CCUGGACC | 224 | GGUCCAGG CUGAUGAGGCCGUUAGGCCGAA ICAGCGUG | 1249 |
| 421 | CGCUGCAC C UGGACCGC | 225 | GCGGUCCA CUGAUGAGGCCGUUAGGCCGAA IUGCAGCG | 1250 |
| 422 | GCUGCACC U GGACCGCU | 226 | AGCGGUCC CUGAUGAGGCCGUUAGGCCGAA IGUGCAGC | 1251 |
| 427 | ACCUGGAC C GCUGCGGC | 227 | GCCGCAGC CUGAUGAGGCCGUUAGGCCGAA IUCCAGGU | 1252 |
| 430 | UGGACCGC U GCGGCCUG | 228 | CAGGCCGC CUGAUGAGGCCGUUAGGCCGAA ICGGUCCA | 1253 |
| 436 | GCUGCGGC C UGCAGGAG | 229 | CUCCUGCA CUGAUGAGGCCGUUAGGCCGAA ICCGCAGC | 1254 |
| 437 | CUGCGGCC U GCAGGAGC | 230 | GCUCCUGC CUGAUGAGGCCGUUAGGCCGAA IGCCGCAG | 1255 |
| 440 | CGGCCUGC A GGAGCUGG | 231 | CCAGCUCC CUGAUGAGGCCGUUAGGCCGAA ICAGGCCG | 1256 |
| 446 | GCAGGAGC U GGGCCCGG | 232 | CCGGGCCC CUGAUGAGGCCGUUAGGCCGAA ICUCCUGC | 1257 |
| 451 | AGCUGGGC C CGGGGCUG | 233 | CAGCCCCG CUGAUGAGGCCGUUAGGCCGAA ICCCAGCU | 1258 |
| 452 | GCUGGGCC C GGGGCUGU | 234 | ACAGCCCC CUGAUGAGGCCGUUAGGCCGAA IGCCCAGC | 1259 |
| 458 | CCCGGGGC U GUUCCGCG | 235 | CGCGGAAC CUGAUGAGGCCGUUAGGCCGAA ICCCCGGG | 1260 |
| 463 | GGCUGUUC C GCGGCCUG | 236 | CAGGCCGC CUGAUGAGGCCGUUAGGCCGAA IAACAGCC | 1261 |
| 469 | UCCGCGGC C UGGCUGCC | 237 | GGCAGCCA CUGAUGAGGCCGUUAGGCCGAA ICCGCGGA | 1262 |
| 470 | CCGCGGCC U GGCUGCCC | 238 | GGGCAGCC CUGAUGAGGCCGUUAGGCCGAA IGCCGCGG | 1263 |
| 474 | GGCCUGGC U GCCCUGCA | 239 | UGCAGGGC CUGAUGAGGCCGUUAGGCCGAA ICCAGGCC | 1264 |
| 477 | CUGGCUGC C CUGCAGUA | 240 | UACUGCAG CUGAUGAGGCCGUUAGGCCGAA ICAGCCAG | 1265 |
| 478 | UGGCUGCC C UGCAGUAC | 241 | GUACUGCA CUGAUGAGGCCGUUAGGCCGAA IGCAGCCA | 1266 |
| 479 | GGCUGCCC U GCAGUACC | 242 | GGUACUGC CUGAUGAGGCCGUUAGGCCGAA IGGCAGCC | 1267 |
| 482 | UGCCCUGC A GUACCUCU | 243 | AGAGGUAC CUGAUGAGGCCGUUAGGCCGAA ICAGGGCA | 1268 |
| 487 | UGCAGUAC C UCUACCUG | 244 | CAGGUAGA CUGAUGAGGCCGUUAGGCCGAA IUACUGCA | 1269 |
| 488 | GCAGUACC U CUACCUGC | 245 | GCAGGUAG CUGAUGAGGCCGUUAGGCCGAA IGUACUGC | 1270 |
| 490 | AGUACCUC U ACCUCCAG | 246 | CUGCAGGU CUGAUGAGGCCGUUAGGCCGAA IAGGUACU | 1271 |
| 493 | ACCUCUAC C UGCAGGAC | 247 | GUCCUGCA CUGAUGAGGCCGUUAGGCCGAA IUAGAGGU | 1272 |
| 494 | CCUCUACC U GCAGGACA | 248 | UGUCCUGC CUGAUGAGGCCGUUAGGCCGAA IGUAGAGG | 1273 |
| 497 | CUACCUGC A GGACAACG | 249 | CGUUGUCC CUGAUGAGGCCGUUAGGCCGAA ICAGGUAG | 1274 |
| 502 | UCCAGGAC A ACGCGCUG | 250 | CAGCGCGU CUGAUGAGGCCGUUAGGCCGAA IUCCUGCA | 1275 |
| 509 | CAACGCGC U GCAGGCAC | 251 | GUGCCUGC CUGAUGAGGCCGUUAGGCCGAA ICGCGUUG | 1276 |
| 512 | CUCUCUOC A GGCACUGC | 252 | GCAGUGCC CUGAUGAGGCCGUUAGGCCGAA ICAGCGCG | 1277 |
| 516 | CUGCAGGC A CUGCCUGA | 253 | UCAGGCAG CUGAUGAGGCCGUUAGGCCGAA ICCUGCAG | 1278 |
| 518 | UCAGUCAC U GCCUGAUG | 254 | CAUCAGUC CUGAUGAGGCCGUUAGGCCGAA IUGCCUGC | 1279 |
| 521 | GGCACUGC C UGAUGACA | 255 | UGUCAUCA CUGAUGAGGCCGUUAGGCCGAA ICAGUGCC | 1280 |
| 522 | GCACUGCC U GAUGACAC | 256 | GUGUCAUC CUGAUGAGGCCGUUAGGCCGAA IGCAGUGC | 1281 |
| 529 | CUGAUGAC A CCUUCCGC | 257 | GCGGAAGG CUGAUGAGGCCGUUAGGCCGAA IUCAUCAG | 1282 |
| 531 | GAUGACAC C UUCCGCGA | 258 | UCGCGGAA CUGAUGAGGCCGUUAGGCCGAA IUGUCAUC | 1283 |
| 532 | AUGACACC U UCCGCGAC | 259 | GUCGCGGA CUGAUGAGGCCGUUAGGCCGAA IGUGUCAU | 1284 |
| 535 | ACACCUUC C GCGACCUG | 260 | CAGGUCGC CUGAUGAGGCCGUUAGGCCGAA IAAGGUGU | 1285 |
| 541 | UCCGCGAC C UGGGCAAC | 261 | GUUGCCCA CUGAUGAGGCCGUUAGGCCGAA IUCGCGGA | 1286 |
| 542 | CCGCGACC U GGGCAACC | 262 | GGUUGCCC CUGAUGAGGCCGUUAGGCCGAA IGUCGCGG | 1287 |
| 547 | ACCUGGUC A ACCUCACA | 263 | UGUGAGGU CUGAUGAGGCCGUUAGGCCGAA ICCCAGGU | 1288 |
| 550 | UGGGCAAC C UCACACAC | 264 | GUGUGUGA CUGAUGAGGCCGUUAGGCCGAA IUUGCCCA | 1289 |
| 551 | GGGCAACC U CACACACC | 265 | GGUGUGUG CUGAUGAGGCCGUUAGGCCGAA IGUUGCCC | 1290 |
| 553 | GCAACCUC A CACACCUC | 266 | GAGGUGUG CUGAUGAGGCCGUUAGGCCGAA IAGGUUGC | 1291 |
| 555 | AACCUCAC A CACCUCUG | 267 | AAGAGGUG CUGAUGAGGCCGUUAGGCCGAA IUGAGGUU | 1292 |
| 557 | CCUCACAC A CCUCUUCC | 268 | GGAAGAGG CUGAUGAGGCCGUUAGGCCGAA IUGUGAGG | 1293 |
| 559 | UCACACAC C UCUUCCUG | 269 | CAGGAAGA CUGAUGAGGCCGUUAGGCCGAA IUGUGUGA | 1294 |
| 550 | CACACACC U CUUCCUGC | 270 | GCAGGAAG CUGAUGAGGCCGUUAGGCCGAA IGUGUGUG | 1295 |
| 562 | CACACCUC U UCCUGCAC | 271 | GUGCAGGA CUGAUGAGGCCGUUAGGCCGAA IAGGUGUG | 1296 |
| 565 | ACCUCUUC C UGCACGGC | 272 | GCCGUGCA CUGAUGAGGCCGUUAGGCCGAA IAAGAGGU | 1297 |
| 566 | CCUCUUCC U GCACGGCA | 273 | UGCCGUGC CUGAUGAGGCCGUUAGGCCGAA IGAAGAGG | 1298 |
| 577 | ACGGCAAC C GCAUCUCC | 274 | GGAGAUGC CUGAUGAGGCCGUUAGGCCGAA IUUGCCGU | 1299 |
| 580 | GCAACCGC A UCUCCAGC | 275 | GCUGGAGA CUGAUGAGGCCGUUAGGCCGAA ICGGUUGC | 1300 |
| 583 | ACCGCAUC U CCAGCGUG | 276 | CACGCUGG CUGAUGAGGCCGUUAGGCCGAA IAUGCGGU | 1301 |
| 585 | CGCAUCUC C AGCGUGCC | 277 | GGCACGCU CUGAUGAGGCCGUUAGGCCGAA IAGAUGCG | 1302 |
| 586 | GCAUCUCC A GCGUGCCC | 278 | GGGCACGC CUGAUGAGGCCGUUAGGCCGAA IGAGAUGC | 1303 |
| 593 | CAGCGUGC C CGAGCGCG | 279 | CGCGCUCG CUGAUGAGGCCGUUAGGCCGAA ICACGCUG | 1304 |
| 594 | AGCGUGCC C GAGCGCGC | 280 | GCGCGCUC CUGAUGAGGCCGUUAGGCCGAA IGCACGCU | 1305 |
| 603 | GAGCGCGC C UUCCGUGG | 281 | CCACGGAA CUGAUGAGGCCGUUAGGCCGAA ICGCGCUC | 1306 |
| 604 | AGCGCGCC U UCCGUGGG | 282 | CCCACGGA CUGAUGAGGCCGUUAGGCCGAA IGCGCGCU | 1307 |
| 607 | GCGCCUUC C GUGGGCUG | 283 | CAGCCCAC CUGAUGAGGCCGUUAGGCCGAA IAAGGCGC | 1308 |
| 614 | CCGUGGGC U GCACAGCC | 284 | GGCUGUGC CUGAUGAGGCCGUUAGGCCGAA ICCCACGG | 1309 |
| 617 | UGGUCUOC A CAGCCUCG | 285 | CGAGGCUG CUGAUGAGGCCGUUAGGCCGAA ICAGCCCA | 1310 |
| 619 | GGCUGCAC A GCCUCGAC | 286 | GUCGAGGC CUGAUGAGGCCGUUAGGCCGAA IUGCAGCC | 1311 |
| 622 | UGCACAGC C UCGACCGU | 287 | ACGGUCGA CUGAUGAGGCCGUUAGGCCGAA ICUGUGCA | 1312 |
| 623 | GCACAGCC U CGACCGUC | 288 | GACGGUCG CUGAUGAGGCCGUUAGGCCGAA IGCUGUGC | 1313 |
| 628 | GCCUCGAC C GUCUCCUA | 289 | UAGGAGAC CUGAUGAGGCCGUUAGGCCGAA IUCGAGGC | 1314 |
| 632 | CGACCGUC U CCUACUGC | 290 | GCAGUAGG CUGAUGAGGCCGUUAGGCCGAA IACGGUCG | 1315 |
| 634 | ACCGUCUC C UACUGCAC | 291 | GUGCAGUA CUGAUGAGGCCGUUAGGCCGAA IAGACGGU | 1316 |
| 635 | CCGUCUCC U ACUGCACC | 292 | GGUGCAGU CUGAUGAGGCCGUUAGGCCGAA IGAGACGG | 1317 |
| 638 | UCUCCUAC U GCACCAGA | 293 | UCUGGUGC CUGAUGAGGCCGUUAGGCCGAA IUAGGAGA | 1318 |
| 641 | CCUACUGC A CCAGAACC | 294 | GGUUCUGG CUGAUGAGGCCGUUAGGCCGAA ICAGUAGG | 1319 |
| 643 | UACUGCAC C AGAACCGC | 295 | GCGGUUCU CUGAUGAGGCCGUUAGGCCGAA IUGCAGUA | 1320 |
| 644 | ACUGCACC A GAACCGCG | 296 | CGCGGUUC CUGAUGAGGCCGUUAGGCCGAA IGUGCAGU | 1321 |
| 649 | ACCAGAAC C GCGUGGCC | 297 | GGCCACGC CUGAUGAGGCCGUUAGGCCGAA IUUCUGGU | 1322 |
| 657 | CGCGUGGC C CAUGUGCA | 298 | UGCACAUG CUGAUGAGGCCGUUAGGCCGAA ICCACGCG | 1323 |
| 658 | GCGUGCCC C AUGUGCAC | 299 | GUCCACAU CUGAUGAGGCCGUUAGGCCGAA IGCCACGC | 1324 |
| 659 | CGUGGCCC A UGUGCACC | 300 | GGUGCACA CUGAUGAGGCCGUUAGGCCGAA IGGCCACG | 1325 |
| 665 | CCAUGUGC A CCCGCAUG | 301 | CAUGCGGG CUGAUGAGGCCGUUAGGCCGAA ICACAUGG | 1326 |
| 667 | AUGUGOAC C CGCAUGCC | 302 | GGCAUGCG CUGAUGAGGCCGUUAGGCCGAA IUGCACAU | 1327 |
| 668 | UGUGCACC C GCAUGCCU | 303 | AGGCAUGC CUGAUGAGGCCGUUAGGCCGAA IGUGCACA | 1328 |
| 671 | GCACCCGC A UGCCUUCC | 304 | GGAAGGCA CUGAUGAGGCCGUUAGGCCGAA ICGGGUGC | 1329 |
| 675 | CCGCAUGC C UUCCGUGA | 305 | UCACGGAA CUGAUGAGGCCGUUAGGCCGAA ICAUGCGG | 1330 |
| 676 | CGCAUGCC U UCCGUGAC | 306 | GUCACGGA CUGAUCAGGCCGUUAGGCCGAA IGCAUGCG | 1331 |
| 679 | AUGCCUUC C GUGACCUG | 307 | AAGGUCAC CUGAUGAGGCCGUUAGGCCGAA IAAGGCAU | 1332 |
| 685 | UCCGUGAC C UUGGCCGC | 308 | GCGGCCAA CUGAUGAGGCCGUUAGGCCGAA IUCACGGA | 1333 |
| 686 | CCGUGACC U UGGCCGCC | 309 | GGCGGCCA CUGAUGAGGCCGUUAGGCCGAA IGUCACGG | 1334 |
| 691 | ACCUUGGC C GOCUCAUG | 310 | CAUGAGGC CUGAUGAGGCCGUUAGGCCGAA ICCAAGGU | 1335 |
| 694 | UUGGCCGC C UCAUGACA | 311 | UGUCAUGA CUGAUGAGGCCGUUAGGCCGAA ICGGCCAA | 1336 |
| 695 | UGGCCGCC U CAUGACAC | 312 | GUGUCAUG CUGAUGAGGCCGUUAGGCCGAA IGCGGCCA | 1337 |
| 697 | GCCGCCUC A UGACACUC | 313 | GAGUGUCA CUGAUGAGGCCGUUAGGCCGAA IAGGCGGC | 1338 |
| 702 | CUCAUGAC A CUCUAUCU | 314 | AGAUAGAG CUGAUGAGGCCGUUAGGCCGAA IUCAUGAG | 1339 |
| 704 | CAUGACAC U CUAUCUGU | 315 | ACAGAUAG CUGAUGAGGCCGUUAGGCCGAA IGGUCAUG | 1340 |
| 706 | UGACACUC U AUCUGUUU | 316 | AAACAGAU CUGAUGAGGCCGUUAGGCCGAA IAGUGUCA | 1341 |
| 710 | ACUCUAUC U GUUUGCCA | 317 | UGGCAAAC CUGAUGAGGCCGUUAGGCCGAA IAUAGAGU | 1342 |
| 717 | CUGUUUGC C AACAAUCU | 318 | AGAUUGUU CUGAUGAGGCCGUUAGGCCGAA ICAAACAG | 1343 |
| 718 | UGUUUGCC A ACAAUCUA | 319 | UAGAUUGU CUGAUGAGGCCGUUAGGCCGAA IGCAAACA | 1344 |
| 721 | UUGCCAAC A AUCUAUCA | 320 | UGAUAGAU CUGAUGAGGCCGUUAGGCCGAA IUUGGCAA | 1345 |
| 725 | CAACAAUC U AUCAGCGC | 321 | GCGCUGAU CUGAUGAGGCCGUUAGGCCGAA IAUUGUUG | 1346 |
| 729 | AAUCUAUC A GCGCUGCC | 322 | GGCAGCGC CUGAUGAGGCCGUUAGGCCGAA IAUAGAUU | 1347 |
| 734 | AUCAGCGC U GCCCACUG | 323 | CAGUGGGC CUGAUGAGGCCGUUAGGCCGAA ICGCUGAU | 1348 |
| 737 | AGCGCUGC C CACUGAGO | 324 | CCUCAGUG CUGAUGAGGCCGUUAGGCCGAA ICAGCGCU | 1349 |
| 736 | GCGCUGCC C ACUGAGGC | 325 | GCCUCAGU CUGAUGAGGCCGUUAGGCCGAA IGCAGCGC | 1350 |
| 739 | CGCUGCCC A CUGAGGCC | 326 | GGCCUCAG CUGAUGAGGCCGUUAGGCCGAA IGGCAGCG | 1351 |
| 741 | CUGCCCAC U GAGGCCCU | 327 | AGGGCCUC CUGAUGAGGCCGUUAGGCCGAA IUGGOCAG | 1352 |
| 747 | ACUGAGGC C CUGGCCCC | 328 | GGGOCCAG CUGAUGAGGCCGUUAGGCCGAA ICCUCAGU | 1353 |
| 748 | CUGAGGCC C UGGCCCCC | 329 | GGGGGCCA CUGAUGAGGCCGUUAGGCCGAA IGCCUCAG | 1354 |
| 749 | UGAGGCCC U GGCCCCCC | 330 | GGGGGGCC CUGAUGAGGCCGUUAGGCCGAA IGGCCUCA | 1355 |
| 753 | GCCCUGGC C CCCCUGCG | 331 | CGCAGGGG CUGAUGAGGCCGUUAGGCCGAA ICCAGGGC | 1356 |
| 754 | CCCUGGCC C CCCUGCGU | 332 | ACGCAGGG CUGAUGAGGCCGUUAGGCCGAA IGCCAGGG | 1357 |
| 755 | CCUGGCCC C CCUGCGUG | 333 | CACGCAGG CUGAUGAGGCCGUUAGGCCGAA IGGCCAGG | 1358 |
| 756 | CUGGCCCC C CUGCGUGC | 334 | GCACGCAG CUGAUGAGGCCGUUAGGCCGAA IGGGCCAG | 1359 |
| 757 | UGGCCCCC C UGCGUGCC | 335 | GGCACGCA CUGAUGAGGCCGUUAGGCCGAA IGGGGCCA | 1360 |
| 758 | GGCCCCCC U GCGUGCCC | 336 | GGGCACGC CUGAUGAGGCCGUUAGGCCGAA IGGGGGCC | 1361 |
| 765 | CUGCGUGC C CUGCAGUA | 337 | UACUGCAG CUGAUGAGGCCGUUAGGCCGAA ICACGCAG | 1362 |
| 766 | UGCGUGCC C UGCAGUAC | 338 | GUACUGCA CUGAUGAGGCCGUUAGGCCGAA IGCACGCA | 1363 |
| 767 | GCGUGCCC U GCAGUACC | 339 | GGUACUGC CUGAUGAGGCCGUUAGGCCGAA IGGCACGC | 1364 |
| 770 | UGCCCUGC A GUACCUGA | 340 | UCAGGUAC CUGAUGAGGCCGUUAGGCCGAA ICAGGGCA | 1365 |
| 775 | UGCAGUAC C UGAGGCUC | 341 | GAGCCUCA CUGAUGAGGCCGUUAGGCCGAA IUACUGCA | 1366 |
| 776 | GCAGUACC U GAGGCUCA | 342 | UGAGCCUC CUGAUGAGGCCGUUAGGCCGAA IGUACUGC | 1367 |
| 782 | CCUGAGGC U CAACGACA | 343 | UGUCGUUG CUGAUGAGGCCGUUAGGCCGAA ICCUCAGG | 1368 |
| 784 | UGAGGCUC A ACGACAAC | 344 | GUUGUCGU CUGAUGAGGCCGUUAGGCCGAA IAGOCUCA | 1369 |
| 790 | UCAACGAC A ACCCCUGG | 345 | CCAGGGGU CUGAUGAGGCCGUUAGGCCGAA IUCGUUGA | 1370 |
| 793 | ACGACAAC C CCUGGGUG | 346 | CACCCAGG CUGAUGAGGCCGUUAGGCCGAA IUUGUCGU | 1371 |
| 794 | CGACAACC C CUGGGUGU | 347 | ACACCCAG CUGAUGAGGCCGUUAGGCCGAA IGUUGUCG | 1372 |
| 795 | GACAACCC C UGGGUGUG | 348 | CACACCCA CUGAUGAGGCCGUUAGGCCGAA IGGUUGUC | 1373 |
| 796 | ACAACCCC U GGGUGUGU | 349 | ACACACCC CUGAUGAGGCCGUUAGGCCGAA IGGGUUGU | 1374 |
| 808 | UGUGUGAC U GCCGGGCA | 350 | UGCCCGGC CUGAUGAGGCCGUUAGGCCGAA IUCACACA | 1375 |
| 811 | GUGACUGC C GGGCACGC | 351 | GCGUGCCC CUGAUGAGGCCGUUAGGCCGAA ICAGUCAC | 1376 |
| 816 | UGCCGGGC A CGCCCACU | 352 | AGUGGGCG CUGAUGAGGCCGUUAGGCCGAA ICCCGGCA | 1377 |
| 820 | GGGCACGC C CACUCUOG | 353 | CCAGAGUG CUGAUGAGGCCGUUAGGCCGAA ICGUGCCC | 1378 |
| 821 | GGCACGCC C ACUCUGOG | 354 | CCCAGAGU CUGAUGAGGCCGUUAGGCCGAA IGCGUGCC | 1379 |
| 822 | GCACGCCC A CUCUGGGC | 355 | GCCCAGAG CUGAUGAGGCCGUUAGGCCGAA IGGCGUGC | 1380 |
| 824 | ACGCCCAC U CUGGGCCU | 356 | AGGCCCAG CUGAUGAGGCCGUUAGGCCGAA IUGGGCGU | 1381 |
| 826 | GCCCACUC U GGGCCUGG | 357 | CCAGGCCC CUGAUGAGGCCGUUAGGCCGAA IAGUGGGC | 1382 |
| 831 | CUCUGGGC C UGGCUGCA | 358 | UGCAGCCA CUGAUGAGGCCGUUAGGCCGAA ICCCAGAG | 1383 |
| 832 | UCUGGGCC U GGCUGCAG | 359 | CUGCAGCC CUGAUGAGGCCGUUAGGCCGAA IGCCCAGA | 1384 |
| 838 | GCCCUGGC U GCAGAAGU | 360 | ACUUCUGC CUGAUGAGGCCGUUAGGCCGAA ICCAGGCC | 1385 |
| 839 | CUGGCUGC A GAAGUUCC | 361 | GGAACUUC CUGAUGAGGCCGUUAGGCCGAA ICAUCCAG | 1386 |
| 847 | AGAAGUUC C GCGGCUCC | 362 | GGAGCCGC CUGAUGAGGCCGUUAGGCCGAA IAACUUCU | 1387 |
| 853 | UCCGCGGC U CCUCCUCC | 363 | GGAGGAGG CUGAUGAGGCCGUUAGGCCGAA ICCGCGGA | 1388 |
| 855 | CGCGGCUC C UCCUCCGA | 364 | UCUGAGGA CUGAUGAGGCCGUUAGGCCGAA IAGCCGCG | 1389 |
| 856 | GCGGCUCC U CCUCCGAG | 365 | CUCGGAGG CUGAUGAGGCCGUUAGGCCGAA IGAGCCGC | 1390 |
| 858 | GGCUCCUC C UCCGAGGU | 366 | ACCUCGGA CUGAUGAGGCCGUUAGGCCGAA IAGGAGCC | 1391 |
| 859 | GCUCCUCC U CCGAGGUG | 367 | CACCUCGG CUGAUGAGGCCGUUAGGCCGAA IGAGGAGC | 1392 |
| 861 | UCCUCCUC C GAGGUGCC | 368 | GGCACCUC CUGAUGAGGCCGUUAGGCCGAA IAGGAGGA | 1393 |
| 869 | CGAGGUGC C CUGCAGCC | 369 | GGCUGCAG CUGAUGAGGCCGUUAGGCCGAA ICACCUCG | 1394 |
| 870 | GAGGUGCC C UGCAGCCU | 370 | AGGCUGCA CUGAUGAGGCCGUUAGGCCGAA IGOACCUC | 1395 |
| 871 | AGGUGCCC U GCAGCCUC | 371 | GAGGCUGC CUGAUGAGGCCGUUAGGCCGAA IGGCACCU | 1396 |
| 874 | UGCCCUGC A GCCUCCCG | 372 | CGGGAGGC CUGAUGAGGCCGUUAGGCCGAA ICAGGGCA | 1397 |
| 877 | CCUGCAGC C UCCCGCAA | 373 | UUGCGGGA CUGAUGAGGCCGUUAGGCCGAA ICUGCAGG | 1398 |
| 878 | CUGCAGCC U CCCGCAAC | 374 | GUUGCGGG CUGAUGAGGCCGUUAGGCCGAA IGCUGCAG | 1399 |
| 880 | GCAGCCUC C CGCAACGC | 375 | GCGUUGCG CUGAUGAGGCCGUUAGGCCGAA IAGGCUGC | 1400 |
| 881 | CAGCCUCC C GCAACGCC | 376 | GGCGUUGC CUGAUGAGGCCGUUAGGCCGAA IGAGGCUG | 1401 |
| 884 | CCUCCCGC A ACGCCUGG | 377 | CCAGGCGU CUGAUGAGGCCGUUAGGCCGAA ICGGGAGG | 1402 |
| 889 | CGCAACGC C UGGCUGGC | 378 | GCCAGCCA CUGAUGAGGCCGUUAGGCCGAA ICGUUGCG | 1403 |
| 890 | GCAACGCC U GGCUGGCC | 379 | GGCCAGCC CUGAUGAGGCCGUUAGGCCGAA IGCGUUGC | 1404 |
| 894 | CGCCUGGC U GGCCGUGA | 380 | UCACGGCC CUGAUGAGGCCGUUAGGCCGAA ICCACGCG | 1405 |
| 898 | UGGCUGGC C GUGACCUC | 381 | GAGGUCAC CUGAUGAGGCCGUUAGGCCGAA ICCAGCCA | 1406 |
| 904 | GCCGUGAC C UCAAACGC | 382 | GCGUUUGA CUGAUGAGGCCGUUAGGCCGAA IUCACGGC | 1407 |
| 905 | CCCUGACC U CAAACGCC | 383 | GGCGUUUG CUGAUGAGGCCGUUAGGCCGAA IGUCACGG | 1408 |
| 907 | GUGACCUC A AACGCCUA | 384 | UAGGCGUU CUGAUGAGGCCGUUAGGCCGAA TAGGUCAC | 1409 |
| 913 | UCAAACGC C UAGCUGCC | 385 | GGCAGCUA CUGAUGAGGCCGUUAGGCCGAA ICGUUUGA | 1410 |
| 914 | CAAACGCC U AGCUGCCA | 386 | UGGCAGCU CUGAUGAGGCCGUUAGGCCGAA IGCGUUUG | 1411 |
| 918 | CGCCUAGC U GCCAAUGA | 387 | UCAUUGGC CUGAUGAGGCCGUUAGGCCGAA ICUAGGCG | 1412 |
| 921 | CUAGCUGC C AAUGACCU | 388 | AGGUCAUC CUGAUGAGGCCGUUAGGCCGAA ICAGCUAG | 1413 |
| 922 | UAGCUGCC A AUGACCUG | 389 | CAGGUCAU CUGAUGAGGCCGUUAGGCCGAA IGCAGCUA | 1414 |
| 928 | CCAAUGAC C UGCAGGGC | 390 | GCCCUGCA CUGAUGAGGCCGUUAGGCCGAA IUCAUUGG | 1415 |
| 929 | CAAUGACC U GCAGGGCU | 391 | AGCCCUGC CUGAUGAGGCCGUUAGGCCGAA IGUCAUUG | 1416 |
| 932 | UGACCUOC A GGGCUGCG | 392 | CGCAGCCC CUGAUGAGGCCGUUAGGCCGAA ICAGGUCA | 1417 |
| 937 | UGCAGGGC U GCGCUGUG | 393 | CACAGCGC CUGAUGAGGCCGUUAGGCCGAA ICCCUGCA | 1418 |
| 942 | GGCUGCGC U GUGGCCAC | 394 | GUGGCCAC CUGAUGAGGCCGUUAGGCCGAA ICGCAGCC | 1419 |
| 948 | GCUGUGGC C ACCGGCCC | 395 | GGGCCGGU CUGAUGAGGCCGUUAGGCCGAA ICCACAGC | 1420 |
| 949 | CUGUGGCC A CCGGCCCU | 396 | AGGGCCGG CUGAUGAGGCCGUUAGGCCGAA IGCCACAG | 1421 |
| 951 | GUGGCCAC C GGCCCCUA | 397 | UAAGGGCC CUGAUGAGGCCGUUAGGCCGAA IUGGCCAC | 1422 |
| 955 | CCACCGGC C CUUACCAU | 398 | AUGGUAAG CUGAUGAGGCCGUUAGGCCGAA ICCGGUGG | 1423 |
| 956 | CACCGGCC C UUACCAUC | 399 | GAUGGUAA CUGAUGAGGCCGUUAGGCCGAA IGCCGGUG | 1424 |
| 957 | ACCGGCCC U UACCAUCC | 400 | GGAUGGUA CUGAUGAGGCCGUUAGGCCGAA IGGCCGGU | 1425 |
| 961 | GCCCUUAC C AUCCCAUC | 401 | GAUGGGAU CUGAUGAGGCCGUUAGGCCGAA IUAAGGGC | 1426 |
| 962 | CCCUUACC A UCCCAUCU | 402 | AGAUGGGA CUGAUGAGGCCGUUAGGCCGAA IGUAAGGG | 1427 |
| 965 | UUACCAUC C CAUCUGGA | 403 | UCCAGAUG CUGAUGAGGCCGUUAGGCCGAA IAUGGUAA | 1428 |
| 966 | UACCAUCC C AUCUGGAC | 404 | GUCCAGAU CUGAUGAGGCCGUUAGGCCGAA IGAUGGUA | 1429 |
| 967 | ACCAUCCC A UCUGGACC | 405 | GGUCCAGA CUGAUGAGGCCGUUAGGCCGAA IGGAUGGU | 1430 |
| 970 | AUCCCAUC U GGACCGGC | 406 | GCCGGUCC CUGAUCAGGCCGUUAGGCCGAA IAUGGGAU | 1431 |
| 975 | AUCUGGAC C GGCAGGGC | 407 | GCCCUGCC CUGAUGAGGCCGUUAGGCCGAA IUCCAGAU | 1432 |
| 979 | GGACCGGC A GGGCCACC | 408 | GGUGGCCC CUGAUGAGGCCGUUAGGCCGAA ICCGGUCC | 1433 |
| 984 | GGCAGGGC C ACCGAUGA | 409 | UCAUCGGU CUGAUGAGGCCGUUAGGCCGAA TCCCUGCC | 1434 |
| 985 | GCAGGGCC A CCGAUGAG | 410 | CUCAUCOG CUGAUGAGGCCGUUAGGCCGAA IGCCCUGC | 1435 |
| 987 | AGGGCCAC C GAUGAGGA | 411 | UCCUCAUC CUGAUGAGGCCGUUAGGCCGAA IUGGCCCU | 1436 |
| 998 | UGAGGAGC C GCUGGGGC | 412 | GCCCCAGC CUGAUGAGGCCGUUAGGCCGAA ICUCCUCA | 1437 |
| 1001 | GGAGCCGC U GGGGCUUC | 413 | GAAGCCCC CUGAUGAGGCCGUUAGGCCGAA ICGGCUCC | 1438 |
| 1007 | GCUGGGGC U UCCCAAGU | 414 | ACUUGGGA CUGAUGAGGCCGUUAGGCCGAA ICCCCAGC | 1439 |
| 1010 | GGGGCUUC C CAAGUGCU | 415 | AGCACUUG CUGAUGAGGCCGUUAGGCCGAA IAAGCCCC | 1440 |
| 1011 | GGGCUUCC C AAGUGCUG | 416 | CAGCACUU CUGAUGAGGCCGUUAGGCCGAA IGAAGCCC | 1441 |
| 1012 | GGCUUCCC A AGUGCUGC | 417 | GCAGCACU CUGAUGAGGCCGUUAGGCCGAA IGGAAGCC | 1442 |
| 1018 | CCAAGUGC U GCCAGCCA | 418 | UGGCUGGC CUGAUGAGGCCGUUAGGCCGAA ICACUUGG | 1443 |
| 1021 | AGUGCUGC C AGCCAGAU | 419 | AUCUGGCU CUGAUGAGGCCGUUAGGCCGAA ICAGCACU | 1444 |
| 1022 | GUGCUGCC A GCCAGAUG | 420 | CAUCUGGC CUGAUGAGGCCGUUAGGCCGAA IGCAGCAC | 1445 |
| 1025 | CUGCCAGC C AGAUGCCG | 421 | CGGCAUCU CUGAUGAGGCCGUUAGGCCGAA ICUGGCAG | 1446 |
| 1026 | UGCCAGCC A GAUGCCGC | 422 | GCGGCAUC CUGAUGAGGCCGUUAGGCCGAA IGCUGGCA | 1447 |
| 1032 | CCAGAUGC C GCUGACAA | 423 | UUGUCAGC CUGAUGAGGCCGUUAGGCCGAA ICAUCUGG | 1448 |
| 1035 | GAUGCCGC U GACAAGGC | 424 | GCCUUGUC CUGAUGAGGCCGUUAGGCCGAA ICGGCAUC | 1449 |
| 1039 | CCGCUGAC A AGGCCUCA | 425 | UGAGGCCU CUGAUGAGGCCGUUAGGCCGAA IUCAGCGG | 1450 |
| 1044 | GACAAGGC C UCAGUACU | 426 | AGUACUGA CUGAUGAGGCCGUUAGGCCGAA ICCUUGUC | 1451 |
| 1045 | ACAAGGCC U CAGUACUG | 427 | CAGUACUG CUGAUGAGGCCGUUAGGCCGAA IGCCUUGU | 1452 |
| 1047 | AAGGCCUC A GUACUGGA | 428 | UCCAGUAC CUGAUGAGGCCGUUAGGCCGAA IAGGCCUU | 1453 |
| 1052 | CUCAGUAC U GGAGCCUG | 429 | CAGGCUCC CUGAUGAGGCCGUUAGGCCGAA IUACUGAG | 1454 |
| 1058 | ACUGGAGC C UGGAAGAC | 430 | GUCUUCCA CUGAUGAGGCCGUUAGGCCGAA ICUCCAGU | 1455 |
| 1059 | CUGGAGCC U GGAAGACC | 431 | GGUCUUCC CUGAUGAGGCCGUUAGGCCGAA IGCUCCAG | 1456 |
| 1067 | UGGAAGAC C AGCUUCGG | 432 | CCGAAGCU CUGAUGAGGCCGUUAGGCCGAA IUCUUCCA | 1457 |
| 1068 | GGAAGACC A GCUUCGGC | 433 | GCCGAAGC CUGAUGAGGCCGUUAGGCCGAA IGUCUUCC | 1458 |
| 1071 | AGACCAGC U UCGGCAGG | 434 | CCUGCCGA CUGAUGAGGCCGUUAGGCCGAA ICUGGUCU | 1459 |
| 1077 | GCUUCGGC A GGCAAUGC | 435 | GCAUUGCC CUGAUGAGGCCGUUAGGCCGAA ICCGAAGC | 1460 |
| 1081 | CGGCAGGC A AUGCGCUG | 436 | CAGCGCAU CUGAUGAGGCCGUUAGGCCGAA ICCUGCCG | 1461 |
| 1088 | CAAUGCGC U GAAGGGAC | 437 | GUCCCUUC CUGAUGAGGCCGUUAGGCCGAA ICGCAUUG | 1462 |
| 1103 | ACGCGUGC C GCCCGGUG | 438 | CACCGGGC CUGAUGAGGCCGUUAGGCCGAA ICACGCGU | 1463 |
| 1106 | CGUGCCGC C CGGUGACA | 439 | UGUCACCG CUGAUGAGGCCGUUAGGCCGAA ICGGCACG | 1464 |
| 1107 | GUGCCGCC C GGUGACAG | 440 | CUGUCACC CUGAUGAGGCCGUUAGGCCGAA IGCGGCAC | 1465 |
| 1114 | CCGGUGAC A GCCCGCCG | 441 | CGGCGGGC CUGAUGAGGCCGUUAGGCCGAA IUCACCGG | 1466 |
| 1117 | GUGACAGC C CGCCGGGC | 442 | GCCCGGCG CUGAUGAGGCCGUUAGGCCGAA ICUGUCAC | 1467 |
| 1118 | UGACAGCC C GCCGGGCA | 443 | UGCCCGGC CUGAUGAGGCCGUUAGGCCGAA IGCUGUCA | 1468 |
| 1121 | CAGCCCGC C GGGCAACG | 444 | CGUUGCCC CUGAUGAGGCCGUUAGGCCGAA ICGGGCUG | 1469 |
| 1126 | CGCCGGGC A ACGGCUCU | 445 | AGAGCCGU CUGAUGAGGCCGUUAGGCCGAA ICCCGGCG | 1470 |
| 1132 | GCAACGGC U CUGGCCCA | 446 | UGGGCCAG CUGAUGAGGCCGUUAGGCCGAA ICCGUUGC | 1471 |
| 1134 | AACGGCUC U GGCCCACG | 447 | CGUGGGCC CUGAUGAGGCCGUUAGGCCGAA IAGCCGUU | 1472 |
| 1138 | GCUCUGGC C CACGGCAC | 448 | GUGCCGUG CUGAUGAGGCCGUUAGGCCGAA ICCAGAGC | 1473 |
| 1139 | CUCUGGCC C ACGGCACA | 449 | UGUGCCGU CUGAUGAGGCCGUUAGGCCGAA IGCCAGAG | 1474 |
| 1140 | UCUGGCCC A CGGCACAU | 450 | AUGUGCCG CUGAUGAGGCCGUUAGGCCGAA IGGCCAGA | 1475 |
| 1145 | CCCACGGC A CAUCAAUG | 451 | CAUUGAUG CUGAUGAGGCCGUUAGGCCGAA ICCGUGGG | 1476 |
| 1147 | CACGGCAC A UCAAUGAC | 452 | GUCAUUGA CUGAUGAGGCCGUUAGGCCGAA IUGCCGUG | 1477 |
| 1150 | GGCACAUC A AUGACUCA | 453 | UGAGUCAU CUGAUGAGGCCGUUAGGCCGAA IAUGUGUC | 1478 |
| 1156 | UCAAUGAC U CACCCUUU | 454 | AAAGGGUG CUGAUGAGGCCGUUAGGCCGAA IUCAUGGA | 1479 |
| 1158 | AAUGACUC A CCCUUUGG | 455 | CCAAAGGG CUGAUGAGGCCGUUAGGCCGAA IAGUCAUG | 1480 |
| 1160 | UGACUCAC C CUUUGGGA | 456 | UCCCAAAG CUGAUGAGGCCGUUAGGCCGAA IUGAGUCA | 1481 |
| 1161 | GACUCACC C UUUGGGAC | 457 | GUCCCAAA CUGAUGAGGCCGUUAGGCCGAA IGUGAGUC | 1482 |
| 1162 | ACUCACCC U UUGGGACU | 458 | AGUCCCAA CUGAUGAGGCCGUUAGGCCGAA IGGUGAGU | 1483 |
| 1170 | UUUGGGAC U CUGCCUGG | 459 | CCAGGCAG CUGAUGAGGCCGUUAGGCCGAA IUCCCAAA | 1484 |
| 1172 | UGUGACUC U GCCUGGCU | 460 | AGCCAGGC CUGAUGAGGCCGUUAGGCCGAA IAGUCCCA | 1485 |
| 1175 | GACUCUGC C UGGCUCUG | 461 | CAGAUCCA CUGAUGAGGCCGUUAGGCCGAA ICAGAGUC | 1486 |
| 1176 | ACUCUGCC U GGCUCUGC | 462 | GCAGAGCC CUGAUGAGGCCGUUAGGCCGAA IGCAGAGU | 1487 |
| 1180 | UGCCUGGC U CUGCUGAG | 463 | CUCAGCAG CUGAUGAGGCCGUUAGGCCGAA ICCAGGCA | 1488 |
| 1182 | CCUGGCUC U GCUGAGCC | 464 | GGCUCAGC CUGAUGAGGCCGUUAGGCCGAA IAGCCAGG | 1489 |
| 1185 | GGCUCUGC U GAGCCCCC | 465 | GGGGGCUC CUGAUGAGGCCGUUAGGCCGAA ICAGAGCC | 1490 |
| 1190 | UGCUGAGC C CCCGCUCA | 466 | UGAGCGGG CUGAUGAGGCCGUUAGGCCGAA ICUCAGCA | 1491 |
| 1191 | GCUGAGCC C CCGCUCAC | 467 | GUGAGCGG CUGAUGAGGCCGUUAGGCCGAA IGCUCAGC | 1492 |
| 1192 | CUGAGCCC C CGCUCACU | 468 | AGUGAGCG CUGAUGAGGCCGUUAGGCCGAA IGGCUCAG | 1493 |
| 1193 | UGAGCCCC C GCUCACUG | 469 | CAGUGAGC CUGAUGAGGCCGUUAGGCCGAA IGGGCUCA | 1494 |
| 1196 | GCCCCCGC U CACUGCAG | 470 | CUGCAGUG CUGAUGAGGCCGUUAGGCCGAA ICGGGGGC | 1495 |
| 1198 | CCCCGCUC A CUOCAGUG | 471 | CACUGCAG CUGAUGAGGCCGUUAGGCCGAA IAGCGGGG | 1496 |
| 1200 | CCGCUCAC U GCAGUGCG | 472 | CGCACUGC CUGAUGAGGCCGUUAGGCCGAA IUGAGCGG | 1497 |
| 1203 | CUCACUGC A GUGCGGCC | 473 | GGCCGCAC CUGAUGAGGCCGUUAGGCCGAA ICAGUGAG | 1498 |
| 1211 | AGUGCGGC C CGAGGGCU | 474 | AGCCCUCG CUGAUGAGGCCGUUAGGCCGAA ICCGCACU | 1499 |
| 1212 | GUGCGGCC C GAGGGCUC | 475 | GAGCCCUC CUGAUGAGGCCGUUAGGCCGAA IGCCGCAC | 1500 |
| 1219 | CCGAGGGC U CCGAGCCA | 476 | UGGCUCGG CUGAUGAGGCCGUUAGGCCGAA ICCCUCGG | 1501 |
| 1221 | GAGGGCUC C GAGCCACC | 477 | GGUGGCUC CUGAUGAGGCCGUUAGGCCGAA IAGCCCUC | 1502 |
| 1226 | CUCCGAGC C ACCAGGGU | 478 | ACCCUGGU CUGAUGAGGCCGUUAGGCCGAA ICUCGGAG | 1503 |
| 1227 | UCCGAGCC A CCAGGGUU | 479 | AACCCUGG CUGAUGAGGCCGUUAGGCCGAA IGCUCGGA | 1504 |
| 1229 | CGAGCCAC C AGGGUUCC | 480 | GGAACCCU CUGAUGAGGCCGUUAGGCCGAA IUGGCUCG | 1505 |
| 1230 | GAGCCACC A GGGUGCCC | 481 | GGGAACCC CUGAUGAGGCCGUUAGGCCGAA IGUGGCUC | 1506 |
| 1237 | CAGGGUUC C CCACCUCG | 482 | CGAGGUGG CUGAUGAGGCCGUUAGGCCGAA IAACCCUG | 1507 |
| 1238 | AGGGUUCC C CACCUCGG | 483 | CCGAGGUG CUGAUGAGGCCGUUAGGCCGAA IGAACCCU | 1508 |
| 1239 | GGGUUCCC C ACCUCGGG | 484 | CCCGAGGU CUGAUGAGGCCGUUAGGCCGAA IGGAACCC | 1509 |
| 1240 | GGUUCCCC A CCUCGGGC | 485 | GCCCGAGG CUGAUGAGGCCGUUAGGCCGAA IGGGAACC | 1510 |
| 1242 | UUCCCCAC C UCGGGCCC | 486 | GGGCCCGA CUGAUGAGGCCGUUAGGCCGAA IUGGGGAA | 1511 |
| 1243 | UCCCCACC U CGGGCCCU | 487 | AGGGCCCG CUGAUGAGGCCGUUAGGCCGAA IGUGGGGA | 1512 |
| 1249 | CCUCGGGC C CUCGCCGG | 488 | CCGGCGAG CUGAUGAGGCCGUUAGGCCGAA ICCCGAGG | 1513 |
| 1250 | CUCGGGCC C UCGCCGGA | 489 | UCCGGCGA CUGAUGAGGCCGUUAGGCCGAA IGCCCGAG | 1514 |
| 1251 | UCGGGCCC U CGCCGGAG | 490 | CUCCGGCG CUGAUGAGGCCGUUAGGCCGAA IGGCCCGA | 1515 |
| 1255 | GCCCUCGC C GGAGGCCA | 491 | UGGCCUCC CUGAUGAGGCCGUUAGGCCGAA ICGAGGGC | 1516 |
| 1262 | CCGGAGGC C AGGCUGUU | 492 | AACAGCCU CUGAUGAGGCCGUUAGGCCGAA ICCUCCGG | 1517 |
| 1263 | CGGAGGCC A GGCUGUUC | 493 | GAACAGCC CUGAUGAGGCCGUUAGGCCGAA IGCCUCCG | 1518 |
| 1267 | GGCCAGGC U GUUCACGC | 494 | GCGUGAAC CUGAUGAGGCCGUUAGGCCGAA ICCUGGCC | 1519 |
| 1272 | GGCUGUUC A CGCAAGAA | 495 | UUCUUGCG CUGAUGAGGCCGUUAGGCCGAA IAACAGCC | 1520 |
| 1276 | GUUCACGC A AGAACCGC | 496 | GCGGUUCU CUGAUGAGGCCGUUAGGCCGAA ICGUGAAC | 1521 |
| 1282 | GCAAGAAC C GCACCCGC | 497 | GCGGGUGC CUGAUGAGGCCGUUAGGCCGAA IUUCUUGC | 1522 |
| 1285 | AGAACCGC A CCCGCAGC | 498 | GCUGCGGG CUGAUGAGGCCGUUAGGCCGAA ICGGUUCU | 1523 |
| 1287 | AACCGCAC C CGCAGCCA | 499 | UGGCUGCG CUGAUGAGGCCGUUAGGCCGAA IUGCGGUU | 1524 |
| 1288 | ACCGCACC C GCAGCCAC | 500 | GUGGCUGC CUGAUGAGGCCGUUAGGCCGAA IGUGCGGU | 1525 |
| 1291 | GCACCCGC A GCCACUGC | 501 | GCAGUGGC CUGAUGAGGCCGUUAGGCCGAA ICGGGUGC | 1526 |
| 1294 | CCCGCAGC C ACUGCCGU | 502 | ACGGCAGU CUGAUGAGGCCGUUAGGCCGAA ICUGCOGG | 1527 |
| 1295 | CCCCAGCC A CUGCCGUC | 503 | GACGCCAG CUGAUGAGGCCGUUAGGCCGAA IGCUGCGG | 1528 |
| 1297 | GCAGCCAC U GCCGUCUG | 504 | CAGACCGC CUGAUGAGGCCGUUAGGCCGAA IUGGCUGC | 1529 |
| 1300 | GCCACUGC C GUCUGGGC | 505 | GCCCAGAC CUGAUGAGGCCGUUAGGCCGAA ICAGUGGC | 1530 |
| 1304 | CUGCCGUC U GGGCCAGG | 506 | CCUGGCCC CUGAUGAGGCCGUUAGGCCGAA IACCGCAG | 1531 |
| 1309 | CUCUGGGC C AGGCAGGC | 507 | GCCUGCCU CUGAUGAGGCCGUUAGGCCGAA ICCCAGAC | 1532 |
| 1310 | UCUGGGCC A GGCAGGCA | 508 | UCCCUGCC CUGAUGAGGCCGUUAGGCCGAA IGCCCAGA | 1533 |
| 1314 | GGCCAGGC A CGCAGCGG | 509 | CCGCUGCC CUGAUGAGGCCGUUAGGCCGAA ICCUGGCC | 1534 |
| 1318 | AGGOAGOC A GCGGGGGU | 510 | ACCCCCGC CUGAUGAGGCCGUUAGGCCGAA ICCUGCCU | 1535 |
| 1335 | GGCGGGAC U GGUGACUC | 511 | GAGUCACC CUGAUGAGGCCGUUAGGCCGAA IUCCCGCC | 1536 |
| 1342 | CUGGUGAC U CAGAAGGC | 512 | GCCUUCUG CUGAUGAGGCCGUUAGGCCGAA IUCACCAG | 1537 |
| 1344 | GGUGACUC A GAAGGCUC | 513 | GAGCCUUC CUGAUGAGGCCGUUAGGCCGAA IAGUCACC | 1538 |
| 1351 | CAGAAGGC U CAGGUGCC | 514 | GGCACCUG CUGAUGAGGCCGUUAGGCCGAA ICCUUCUG | 1539 |
| 1353 | GAAGGCUC A GGUGCCCU | 515 | AGGGCACC CUGAUGAGGCCGUUAGGCCGAA IAGCCUUC | 1540 |
| 1359 | UCAGGUGC C CUACCCAG | 516 | CUGGGUAG CUGAUGAGGCCGUUAGGCCGAA ICACCUGA | 1541 |
| 1360 | CAGGUGCC C UACCCAGC | 517 | GCUGGGUA CUGAUGAGGCCGUUAGGCCGAA IGCACCUG | 1542 |
| 1361 | AGGUGCCC U ACCCAGCC | 518 | GGCUGGGU CUGAUGAGGCCGUUAGGCCGAA IGGCACCU | 1543 |
| 1364 | UGCCCUAC C CAUCCUCA | 519 | UGAGGCUG CUGAUGAGGCCGUUAGGCCGAA IUAGGGCA | 1544 |
| 1365 | GCCCUACC C AGCCUCAC | 520 | GUGAGGCU CUGAUGAGGCCGUUAGGCCGAA IGUAGGGC | 1545 |
| 1366 | CCCUACCC A GCCUCACC | 521 | GGUGAGGC CUGAUGAGGCCGUUAGGCCGAA IGGUAGGG | 1546 |
| 1369 | UACCCAGC C UCACCUGC | 522 | GCAGGUGA CUGAUGAGGCCGUUAGGCCGAA ICUGGGUA | 1547 |
| 1370 | ACCCAGCC U CACCUGCA | 523 | UGCAGGUG CUGAUGAGGCCGUUAGGCCGAA IGCUGGGU | 1548 |
| 1372 | CCAGCCUC A CCUGCAGC | 524 | GCUGCAGG CUGAUGAGGCCGUUAGGCCGAA IAGGCUGG | 1549 |
| 1374 | AGCCUCAC C UGCAGCCU | 525 | AGGCUGCA CUGAUGAGGCCGUUAGGCCGAA IUGAGGCU | 1550 |
| 1375 | GCCUCACC U GCAGCCUC | 526 | GAGGCUGC CUGAUGAGGCCGUUAGGCCGAA IGUGAGGC | 1551 |
| 1378 | UCACCUGC A GCCUCACC | 527 | GGUGAGGC CUGAUGAGGCCGUUAGGCCGAA ICAGGUGA | 1552 |
| 1381 | CCUGCAGC C UCACCCCC | 528 | GGGGGUGA CUGAUGAGGCCGUUAGGCCGAA ICUGCAGG | 1553 |
| 1382 | CUGCAGCC U CACCCCCC | 529 | GGGGGGUG CUGAUGAGGCCGUUAGGCCGAA IGCUGCAG | 1554 |
| 1384 | GCAGCCUC A CCCCCCUG | 530 | CAGGGGGG CUGAUGAGGCCGUUAGGCCGAA IAGGCUGC | 1555 |
| 1386 | AGCCUCAC C CCCCUGGG | 531 | CCCAGGGG CUGAUGAGGCCGUUAGGCCGAA IUGAGGCU | 1556 |
| 1387 | GCCUCACC C CCCUGGGC | 532 | GCCCAGGG CUGAUGAGGCCGUUAGGCCGAA IGUGAGOC | 1557 |
| 1388 | CCUCACCC C CCUGGGCC | 533 | GGCCCAGG CUGAUGAGGCCGUUAGGCCGAA IGGUGAGG | 1558 |
| 1389 | CUCACCCC C CUGGGCCU | 534 | AGGCCCAG CUGAUGAGGCCGUUAGGCCGAA IGGGUGAG | 1559 |
| 1390 | UCACCCCC C UGGGCCUG | 535 | CAGGCCCA CUGAUGAGGCCGUUAGGCCGAA IGGGGUGA | 1560 |
| 1391 | CACCCCCC U GGGCCUGG | 536 | CCAGGCCC CUGAUGAGGCCGUUAGGCCGAA IGGGGGUG | 1561 |
| 1396 | CCCUGGGC C UGGCGCUG | 537 | CAGCGCCA CUGAUGAGGCCGUUAGGCCGAA ICCCAGGG | 1562 |
| 1397 | CCUGGGCC U GGCGCUGG | 538 | CCAGCGCC CUGAUGAGGCCGUUAGGCCGAA IGCCCAGG | 1563 |
| 1403 | CCUGGCGC U GGUGCUGU | 539 | ACAGCACC CUGAUGAGGCCGUUAGGCCGAA ICGCCAGG | 1564 |
| 1409 | GCUGGUGC U GUGGACAG | 540 | CUGUCCAC CUGAUGAGGCCGUUAGGCCGAA ICACCAGC | 1565 |
| 1416 | CUGUGGAC A GUGCUUGG | 541 | CCAAGCAC CUGAUGAGGCCGUUAGGCCGAA IUCCACAG | 1566 |
| 1421 | GACAGUGC U UGGGCCCU | 542 | AGGGCCCA CUGAUGAGGCCGUUAGGCCGAA ICACUGUC | 1567 |
| 1427 | GCUUGGGC C CUGCUGAC | 543 | GUCAGCAG CUGAUGAGGCCGUUAGGCCGAA ICCCAAGC | 1568 |
| 1428 | CUUGGGCC C UGCUGACC | 544 | GGUCAGCA CUGAUGAGGCCGUUAGGCCGAA IGCCCAAG | 1569 |
| 1429 | UUGGGCCC U GCUGACCC | 545 | GGGUCAGC CUGAUGAGGCCGUUAGGCCGAA IGGCCCAA | 1570 |
| 1432 | GGCCCUGC U GACCCCCA | 546 | UGGGGGUC CUGAUGAGGCCGUUAGGCCGAA ICAGGGCC | 1571 |

Input Sequence = AB020693. Cut Site = CH/.
Arm Length = 8. Core Sequence = CUGAUGAG GCCGUUAGGC CGAA
AB020693 (Homo sapiens mRNA for K1AA0886 protein (Nogo-A); 4053 bp)
Underlined region can be any X sequence or linker, as described herein. I = Inosine

TABLE V
Human NOGO Receptor Zinzyme Ribozyme and Substrate
Sequence

| | | Seq | | Rz Seq |
|---|---|---|---|---|
| Pos | Substrate | ID | Ribozyme | ID |
| 22 | UGAAGAGG G CGUCCGCU | 547 | AGCGGACG GCCGAAAGGCGAGUGAGGUCU CCUCUUCA | 1572 |
| 24 | AAGAGGGC G UCCGCUGG | 548 | CCAGCGGA GCCGAAAGGCGAGUGAGGUCU GCCCUCUU | 1572 |
| 28 | GGGCGUCC G CUGGAGGG | 549 | CCCUCCAG GCCGAAAGGCGAGUGAGGUCU GGACGCCC | 1574 |
| 38 | UGGAGGGA G CCGGCUGC | 550 | GCAGCCGG GCCGAAAGGCGAGUGAGGUCU UCCCUCCA | 1575 |
| 42 | GGGAGCCG G CUGCUGGC | 551 | GCCAGCAG GCCGAAAGGCCAGUGAGGUCU CGGCUCCC | 1576 |
| 45 | AGCCGGCU G CUGGCAUG | 552 | CAUGCCAC GCCGAAAGGCGACUGAGGUCU ACCCGGCU | 1577 |
| 49 | GCCUCCUC G CAUCCGUG | 553 | CACCCAUG GCCGAAAGGCCAGUGAGGUCU CAGCAGCC | 1578 |
| 55 | UGGCAUCG G UGCUGUGG | 554 | OCACACCA GCCGAAAGGCGAGUGAGGUCU CCAUGCCA | 1579 |
| 57 | GCAUCGGU G CUCUGCCU | 555 | AGCCACAG CCCGAAACGCGAGUCAGGUCU ACCCAUGC | 1580 |
| 60 | UCCGUGCU G UGGCUCCA | 556 | UGCAGCCA GCCGAAAGGCCAGUGACCUCU ACCACOCA | 1581 |
| 63 | GUGCUCUG G CUGCAGGC | 557 | GCCUCCAC GCCGAAACCCCACUCACGUCU CACACCAC | 1582 |
| 66 | CUGUCCCU G CAGGOCUC | 558 | CACGCCUC GCCGAAAGGCCACUGAGGUCU AGCCACAC | 1583 |
| 70 | CGCUGCAG G CCUCCCAG | 559 | CUGCCAGG GCCGAAAGGCGACUCAGGUCU CUGCACCC | 1584 |
| 75 | CAGGCCUG G CAGCUCCC | 560 | GCCACCUC GCCGAAAGCCGAGUGAGGUCU CAGGCCUG | 1585 |
| 79 | CCUCCCAC G UCGCACCC | 561 | CCCUGCCA GCCGAAAGCCGAGUGACCUCU CUCCCACG | 1586 |
| 82 | GGCACGUC G CAGCCCCA | 562 | UGGCGCUG GCCGAAAGGCGAGUGAGCUCU CACCUGCC | 1587 |
| 85 | ACGUGGCA G CCCCAUGC | 563 | CCAUGCGG GCCGAAACCCGAGUGAGGUCU UGCCACCU | 1588 |
| 92 | AGCCCCAU G CCCACCUG | 564 | CACCUCCG GCCGAAAGCCCAGUGAGGUCU AUCCCCCU | 1589 |
| 98 | AUGCCCAG G UCCCUCCG | 565 | CCCAGCCA CCCGAAAGCCGAGUCACCUCU CUCGCCAU | 1590 |
| 100 | GCCCAGGU G CCUCCCUA | 566 | UACCCACG GCCGAAAGGCCACUCACGUCU ACCUGGGC | 1591 |
| 104 | ACCUGCCU G CGUAUCCU | 567 | ACCAUACG GCCGAAAGCCGACUGAGGUCU AGGCACCU | 1592 |
| 106 | CUCCCUGC G UAUGCUAC | 568 | GUACCAUA GCCGAAAGGCCAGUGAGGUCU CCAGGCAC | 1593 |
| 110 | CUCCCUAU G CUACAAUC | 569 | CACUCUAG GCCGAAACCCGAGUGACGUCU AUACGCAC | 1594 |
| 120 | UACAAUCA G CCCAAGGU | 570 | ACCUUCCG GCCGAAAGGCGAGUCAGGUCU UCACUCCA | 1595 |
| 127 | AGCCCAAC G UGACCACA | 571 | UGUCCUCA GCCGAAACCCGAGUGAGGUCU CUUCCGCU | 1596 |
| 137 | CACCACAA G CUCCCCCC | 572 | CCCCCCAC CCCGAAAGCCCACUCACGUCU CUCUCCUC | 1597 |
| 140 | CACAACCU G CCCCCACC | 573 | CCUCCCCC GCCGAAAGGCGACUCACCUCU ACCUUCUC | 1598 |
| 147 | UCCCCCCA G CACCCCCU | 574 | ACCCCCUC GCCGAAACCCGAGUGACCUCU UCGGGGCA | 1599 |
| 152 | CCACCACG G CCUGCACG | 575 | CCUCCAGC GCCGAAAGCCCACUGACCUCU CCUCCUGC | 1600 |
| 156 | CACCCCCU G CACCCUCU | 576 | ACACCCUC GCCGAAACGCCAGUGAGCUCU ACCCCCUG | 1601 |
| 160 | CCCUCCAC G CUCUCCCC | 577 | CCCCACAC GCCGAAACCCGACUCACCUCU CUCCACCC | 1602 |
| 163 | UCCACCCU G UCCCCCUC | 578 | CACCCCCA GCCGAAAGGCGACUCACCUCU ACCCUGCA | 1603 |
| 165 | CACCCUCU G CCCCUCCC | 579 | CCCACCCC GCCGAAAGGCCACUCACGUCU ACACCCUC | 1604 |
| 169 | CUCUCCCC G UCCCCAUC | 580 | CAUCCCCA GCCGAAACGCGACUCACCUCU CCCCACAG | 1605 |
| 173 | CCCCCUCC G CAUCCCUC | 581 | CACCGAUC GCCGAAACCCGAGUCACCUCU CCACCCCC | 1606 |
| 181 | CCAUCCCU G CUCCCACC | 582 | CCUCCCAC GCCGAAAGCCCACUCAGCUCU ACCCAUCC | 1607 |
| 184 | UCCCUCCU G CCACCCAC | 583 | CUCCCUCC GCCGAAACCCCACUCACCUCU ACCACCCA | 1608 |
| 188 | UCCUCCCA G CCACCCCA | 584 | UCCGCUCG GCCGAAACCCCACUCACCUCU UCCCACCA | 1609 |
| 192 | CCCACCCA G CCCAUCUU | 585 | AACAUCCC GCCGAAAGCCCACUCACCUCU UCCCUGGC | 1610 |
| 194 | CACCCACC G CAUCUUCC | 586 | GCAACAUG GCCGAAAGGCGACUCAGGUCU CCUCCCUC | 1611 |
| 204 | AUCUUCCU G CACGGCAA | 587 | UUCCCGUG GCCGAAAGGCGAGUCAGCUCU ACCAAGAU | 1612 |
| 209 | CCUCCACC G CAACCCCA | 588 | UCCCGUUC GCCGAAAGCCGACUGAGGUCU CCUCCACG | 1613 |
| 572 | CCUCCACC G CAACCCCA | 588 | UCCGCUUC GCCGAAAGCCCAGUGACGUCU CCUCCAGG | 1614 |
| 215 | CCCCAACC G CAUCUCCC | 589 | CCCACAUG GCCGAAACCCCACUCACCUCU CCUUGCCG | 1615 |
| 222 | CCCAUCUC G CAUCUCCC | 590 | CCCACAUC GCCGAAACGCGACUCACCUCU CACAUCCC | 1616 |
| 226 | UCUCCCAU G UCCCACCU | 591 | ACCUCCCA GCCGAAAGCCGACUCACCUCU AUCCCACA | 1617 |
| 228 | UCGCAUGU G CCAGCUGC | 592 | GCAGCUGG GCCGAAAGGCGAGUGAGGUCU ACAUGCGA | 1618 |
| 232 | AUGUGCCA G CUGOCAGO | 593 | GCUGGCAG GCCGAAAGGCGAGUGAGGUCU UGGCACAU | 1619 |
| 235 | UGCCAGCU G CCAGCUUC | 594 | GAAGCUGG GCCGAAAGGCGAGUGAGGUCU AGCUGGCA | 1620 |
| 239 | AGCUGCCA G CUUCCGUG | 595 | CACGGAAG GCCGAAAGGCGAGUGAGGUCU UGGCAGCU | 1621 |
| 245 | CAGCUUCC G UGCCUGCC | 596 | GGCAGGCA GCCGAAAGGCGAGUGAGGUCU GGAAGCUG | 1622 |
| 247 | GCUUCCGU G CCUGCCGC | 597 | CCGGCAGG GCCGAAAGGCGAGUGAGGUCU ACGGAACC | 1623 |
| 251 | CCGUGCCU G CCGCAACC | 598 | GGUUGCCG GCCGAAAGCCGAGUGACGUCU ACGCACGC | 1624 |
| 254 | UCCCUGCC G CAACCUCA | 599 | UGACGUUC GCCGAAAGGCGAGUGAGGUCU GGCAGCCA | 1625 |
| 270 | ACCAUCCU G UGGCUCCA | 600 | UCCACCCA GCCCAAACCCCAGUCACGUCU AGGAUGGU | 1626 |
| 273 | AUCCUGUG G CUGCACUC | 601 | GAGUCCAG GCCGAAAGGCGAGUGAGGUCU CACAGGAU | 1627 |
| 276 | CUCUGCCU G CACUCCAA | 602 | UUCGACUG GCCGAAACCCGAGUGAGGUCU AUCCACAC | 1628 |
| 286 | ACUCGAAU G UGCUGCCC | 603 | GGCCAGCA GCCGAAAGGCGAGUGAGGUCU AUUCGAGU | 1629 |
| 288 | UCGAAUGU G CUGGCCCG | 604 | CGGGCCAG GCCGAAAGGCGAGUGAGGUCU ACAUUCCA | 1630 |
| 292 | AUGUCCUC G CCCCAAUU | 605 | AAUUCCGC GCCGAAAGGCCAGUCAGCUCU CAGCACAU | 1631 |
| 304 | GAAUUCAU G CGGCUCCC | 606 | GGCAGCCG GCCGAAAGGCGAGUGAGGUCU AUCAAUUC | 1632 |
| 307 | UUGAUCCC G CUGCCUUC | 607 | GAAGCCAG GCCGAAAGGCGAGUGAGGUCU CGCAUCAA | 1633 |
| 310 | AUGCGGCU G CCUUCACU | 608 | AGUGAAGG GCCGAAAGGCGAGUGAGGUCU AGCCGCAU | 1634 |
| 320 | CUUCACUG G CCUCGCCC | 609 | GCCCCAGG GCCGAAAGGCCAGUGACGUCU CAGUCAAG | 1635 |
| 325 | CUGGCCUG G CCCUCCUG | 610 | CACGAGGG GCCGAAAGGCCAGUGACGUCU CACCCCAG | 1636 |
| 336 | CUCCUCCA G CAGCUCCA | 611 | UCCACCUG GCCGAAAGCCGAGUCAGGUCU UCCAGGAC | 1637 |
| 339 | CUCCAGCA G CUGCACCU | 612 | ACCUCCAC GCCGAAAGGCCACUGACCUCU UCCUCCAC | 1638 |
| 350 | GGACCUCA G CGAUAAUC | 613 | CAUUAUCG GCCGAAAGGCGAGUGAGGUCU UGAGGUCC | 1639 |
| 358 | CCCAUAAU G CACAGCUC | 614 | CACCUCUG GCCGAAAGCCGAGUCACGUCU AUUAUCGC | 1640 |
| 363 | AAUGCACA G CUCCGCUC | 615 | GACCGGAG GCCGAAAGGCGAGUGAGGUCU UGUGCAUU | 1641 |
| 369 | CACCUCCC G UCUCUCCA | 616 | UCCACACA GCCGAAAGGCGAGUGAGCUCU CCCACCUC | 1642 |
| 373 | UCCCCUCU G UCCACCCU | 617 | ACCGUCCA GCCGAAAGCCCAGUGACGUCU ACACCCGA | 1643 |
| 382 | UCCACCCU G CCACAUUC | 618 | CAAUCUCG GCCGAAACCCGAGUCACCUCU ACCCUCCA | 1644 |
| 395 | AUUCCACC G CCUCCGCC | 619 | CCCCCAGC GCCGAAACGCGACUCAGGUCU CCUCGAAU | 1645 |
| 401 | CCCCCUCC G CCCCCUAC | 620 | CUACGCGG GCCGAAACGCGAGUCAGGUCU CCACGCCG | 1646 |
| 404 | CCUCCCCC G CCUACACA | 621 | UCUCUACC GCCGAAAGGCGAGUGAGGUCU CCCCCACG | 1647 |
| 414 | CUACACAC G CUGCACCU | 622 | ACCUCCAG GCCGAAAGGCGAGUGAGGUCU CUCUCUAG | 1648 |
| 417 | CACACCCU G CACCUCCA | 623 | UCCACCUC GCCCAAAGGCCACUGACCUCU ACCCUCUC | 1649 |
| 428 | CCUCCACC G CUGCCCCC | 624 | CCCCCCAG GCCGAAAGGCGAGUCAGGUCU CCUCCAGC | 1650 |
| 431 | CCACCCCU G CCCCCUCC | 625 | CCAGCCCG GCCGAAAGGCGAGUGAGGUCU ACCCGUCC | 1651 |
| 434 | CCCCUCCC G CCUGCACG | 626 | CCUCCAGG GCCGAAAGGCGAGUGAGGUCU CGCACCGG | 1652 |
| 438 | UCCCCCCU G CAGCACCU | 627 | ACCUCCUG GCCGAAAGGCGACUCAGGUCU ACCCCGCA | 1653 |
| 444 | CUCCACCA G CUCCGCCC | 628 | CCCCCCAG GCCGAAAGGCGAGUCACCUCU UCCUCCAG | 1654 |
| 449 | CCAGCUCC G CCCGGCCC | 629 | CCCCCGCG GCCGAAAGGCCAGUGACGUCU CCACCUCC | 1655 |
| 456 | CCCCCCCC G CUCUUCCC | 630 | CCCAACAG GCCGAAAGGCGAGUGAGCUCU CCCCGCCC | 1656 |
| 459 | CCCCGCCU G UUCCCCCC | 631 | CCGCGGAA GCCGAAAGGCGAGUGAGGUCU AGCCCCGG | 1657 |
| 464 | CCUCUUCC G CCCCCUCC | 632 | CCACGCCC GCCGAAAGCCGAGUCACCUCU CCAACACC | 1658 |
| 467 | CUUCCCCC G CCUCCCUC | 633 | CACCCAGG GCCGAAAGCCGAGUCACGUCU CCCCCAAC | 1659 |
| 472 | GCCCCCUG G CUCCCCUC | 634 | CACGGCAG GCCGAAAGGCGAGUGAGGUCU CACCCCGC | 1660 |
| 475 | CCCUCCCU G CCCUCCAG | 635 | CUCCAGGC GCCGAAACCCGAGUCAGGUCU ACCCACGC | 1661 |
| 480 | GCUCCCCU G CAGUACCU | 636 | AGGUACUC GCCGAAAGCCCAGUCAGGUCU AGCGCAGC | 1662 |
| 483 | CCCCUCCA G UACCUCUA | 637 | UACAGCUA GCCGAAAGCCCACUCACGUCU UGCAGGGC | 1663 |
| 495 | CUCUACCU G CACCACAA | 638 | UGGUCCUG GCCGAAACGCCAGUGACCUCU ACCUACAG | 1664 |
| 505 | ACCACAAC G CGCUGCAG | 639 | CUGCAGCG GCCGAAAGGCGAGUGAGGUCU GUUCUCCU | 1665 |
| 507 | CACAACCC G CUGCACCC | 640 | CCCUGCAG GCCGAAAGGCGAGUGAGGUCU GCGUUGUC | 1666 |
| 510 | AACCCCCU G CAGGCACU | 641 | ACUCCCUC GCCGAAAGCCGAGUGAGGUCU ACCGCGUU | 1667 |
| 514 | CCCUCCAC G CACUCCCU | 642 | ACCCAGUG GCCGAAAGCCGACUCACGUCU CUCCAGCG | 1668 |
| 519 | CAGGCACU G CCUGAUGA | 643 | UCAUCAGG GCCGAAAGGCGAGUGAGGUCU AGUGCCUG | 1669 |
| 536 | CACCUUCC G CGACCUGG | 644 | CCAGGUCG GCCGAAAGGCGAGUGAGGUCU GGAAGGUG | 1670 |
| 545 | CGACCUGG G CAACCUCA | 645 | UGAGGUUG GCCGAAAGGCGAGUGAGGUCU CCAGGUCG | 1671 |
| 567 | CUCUUCCU G CACGGCAA | 646 | UUGCCGUG GCCGAAAGGCGAGUGAGGUCU AGCAAGAG | 1672 |
| 578 | CGGCAACC G CAUCUCCA | 647 | UGGAGAUG GCCGAAAGGCGAGUGAGGUCU GGUUGCCG | 1673 |
| 587 | CAUCUCCA G CGUGCCCG | 648 | CGGGCACG GCCGAAAGGCGAGUGAGGUCU UGGAGAUG | 1674 |
| 589 | UCUCCAGC G UGCCCGAG | 649 | CUCCGGCA GCCGAAAGGCGAGUGAGGUCU GCUGGAGA | 1675 |
| 591 | UCCAGCGU G CCCGAGCG | 650 | CGCUCGGG GCCGAAAGGCGAGUGAGGUCU ACGCUGGA | 1676 |
| 597 | GUGCCCGA G CGCGCCUU | 651 | AAGGCGCG GCCGAAAGGCGAGUGAGGUCU UCGGGCAC | 1677 |
| 599 | GCCCGAGC G CGCCUUCC | 652 | GGAACGCG GCCCAAAGGCGAGUCAGGUCU CCUCGGCC | 1678 |
| 601 | CCGACCGC G CCUUCCGU | 653 | ACCGAAGG GCCGAAAGGCGAGUGAGGUCU GCCCUCGG | 1679 |
| 608 | CCCCUUCC G UGGGCUGC | 654 | GCAGCCCA GCCGAAAGGCGACUGAGGUCU GGAAGGCG | 1680 |
| 612 | UUCCGUCC G CUGCACAG | 655 | CUGUGCAG GCCGAAAGGCCAGUCAGCUCU CCACCGAA | 1681 |
| 615 | CGUGGGCU G CACAGCCU | 656 | AGGCUGUG GCCGAAAGGCGAGUGAGGUCU AGCCCACG | 1682 |
| 620 | GCUGCACA G CCUCGACC | 657 | GGUCGAGG GCCGAAAGGCGAGUGAGCUCU UGUGCAGC | 1683 |
| 629 | CCUCGACC G UCUCCUAC | 658 | GUAGGAGA GCCGAAAGGCGAGUGAGGUCU GGUCGAGG | 1684 |
| 639 | CUCCUACU G CACCAGAA | 659 | UUCUGGUG GCCGAAAGGCGAGUGAGGUCU AGUACCAG | 1685 |
| 650 | CCAGAACC G CGUGGCCC | 660 | GGGCCACG GCCGAAAGGCGAGUGACGUCU GGUUCUGG | 1686 |
| 652 | ACAACCGC G UGGCCCAU | 661 | AUGCGCCA GCCGAAAGGCCAGUGAGGUCU GCGGUUCU | 1687 |
| 655 | ACCGCGUG G CCCAUGUG | 662 | CACAUGGG GCCGAAAGGCGAGUGAGGUCU CACGCGGU | 1688 |
| 661 | UGGCCCAU G UGCACCCC | 663 | CGGGUGCA GCCGAAAGGCGAGUGAGGUCU AUGGGCCA | 1689 |
| 663 | GCCCAUGU G CACCCGCA | 664 | UGCGGCUG GCCGAAACGCGAGUGAGGUCU ACAUGGCC | 1690 |
| 669 | CUCCACCC G CAUGCCUU | 665 | AACGCAUG GCCGAAAGGCCACUGAGGUCU CGGUGCAC | 1691 |
| 673 | ACCCGCAU G CCUUCCGU | 666 | ACCGAACG GCCGAAACCCGACUCAGGUCU AUGCCCCU | 1692 |
| 680 | UGCCUUCC G UGACCUUG | 667 | CAAGGUCA GCCCAAAGGCGAGUGACCUCU GCAAGGCA | 1693 |
| 689 | UGACCUUG G CCGCCUCA | 668 | UGACCCCG GCCGAAAGCCGAGUGAGGUCU CAACGUCA | 1694 |
| 692 | CCUUCCCC G CCUCAUCA | 669 | UCAUGACC GCCGAAAGCCCACUGAGCUCU CCCCAACG | 1695 |
| 711 | CUCUAUCU G UUUCCCAA | 670 | UUCCCAAA GCCGAAACGCGAGUGAGCUCU ACAUACAC | 1696 |
| 715 | AUCUCUUU G CCAACAAU | 671 | AUUCUUCC GCCGAAAGCCCACUCACCUCU AAACACAU | 1697 |
| 730 | AUCUAUCA G CCCUCCCC | 672 | CCCCACCC GCCCAAAGCCCAGUCACCUCU UCAUAGAU | 1698 |
| 732 | CUAUCACC G CUCCCCAC | 673 | CUCCCCAC GCCGAAACCCGAGUCACCUCU CCUCAUAC | 1699 |
| 735 | UCACCCCU G CCCACUGA | 674 | UCACUCCC GCCGAAAGGCGAGUGACCUCU AGCGCUCA | 1700 |
| 745 | CCACUGAG G CCCUCGCC | 675 | GCCCAGGG GCCGAAAGGCGAGUGAGGUCU CUCAGUCG | 1701 |
| 751 | ACCCCCUG G CCCCCCUC | 676 | CACGGCCC GCCGAAAGGCCAGUGACGUCU CAGGGCCU | 1702 |
| 759 | CCCCCCCU G CCUGCCCU | 677 | AGGGCACC GCCGAAAGCCGAGUGAGGUCU ACCCCCCC | 1703 |
| 761 | CCCCCUCC G UCCCCUCC | 678 | CCACCCCA GCCGAAAGGCCACUGACCUCU CCACCCCC | 1704 |
| 763 | CCCUCCCU G CCCUCCAC | 679 | CUCCACCC GCCGAAACCCCACUCACCUCU ACCCACCC | 1705 |
| 768 | CCUCCCCU G CACUACCU | 680 | ACCUACUC GCCGAAACGCGAGUCAGCUCU ACCCCACC | 1706 |
| 771 | CCCCUCCA G UACCUCAC | 681 | CUCACCUA GCCGAAAGCCCACUCACGUCU UCCACGCC | 1707 |
| 780 | UACCUCAC G CUCAACCA | 682 | UCCUUCAC GCCGAAACCCCACUCACCUCU CUCACCUA | 1708 |
| 799 | ACCCCUCC G UCUCUCAC | 683 | GUCACACA GCCGAAACCCGAGUCACGUCU CCAGCGGU | 1709 |
| 801 | CCCUCCCU G UCUCACUC | 684 | CACUCACA GCCGAAACCCGACUCACCUCU ACCCACCC | 1710 |
| 803 | CUCCCUCU G UCACUCCC | 685 | CCCAGUCA GCCGAAACCCGAGUCACCUCU ACACCCAC | 1711 |
| 809 | CUCUCACU G CCCCCCAC | 686 | CUCCCCCC GCCGAAACGCCACUCAGCUCU ACUCACAC | 1712 |
| 814 | ACUCCCCC G CACCCCCA | 687 | UCCCCCUC GCCGAAAGCCCACUGAGGUCU CCCCCACU | 1713 |
| 818 | CCCCCCAC G CCCACUCU | 688 | ACACUGCC GCCGAAACCCGAGUGACGUCU CUCCCCCC | 1714 |
| 829 | CACUCUCC G CCUCGCUC | 689 | CACCCAGG GCCGAAACGCGAGUCACCUCU CCACAGUC | 1715 |
| 834 | UCCCCCUC G CUCCACAA | 690 | UUCUGCAC GCCGAAAGGCGAGUGAGCUCU CACCCCCA | 1716 |
| 837 | CCCUCCCU G CACAACUU | 691 | AACUUCUC GCCGAAACCCCAGUCAGCUCU ACCCAGCC | 1717 |
| 843 | CUCCACAA G UUCCCCCC | 692 | CCCCCCAA GCCGAAACCCCACUCACCUCU UUCUCCAC | 1718 |
| 848 | CAACUUCC G CCCCUCCU | 693 | ACCACCCC GCCGAAACCCCACUGACCUCU CCAACUUC | 1719 |
| 851 | GUUCCGCG G CUCCUCCU | 694 | AGGAGGAG GCCGAAAGGCGAGUGAGGUCU CGCGGAAC | 1720 |
| 865 | CCUCCGAG G UGCCCUGC | 695 | GCAGGGCA GCCGAAAGGCGAGUGAGGUCU CUCGGAGG | 1721 |
| 867 | UCCGAGGU G CCCUGCAG | 696 | CUGCAGGG GCCGAAAGGCGAGUGAGGUCU ACCUCOGA | 1722 |
| 872 | GGUGCCCU G CAGCCUCC | 697 | GGAGGCUG GCCGAAAGGCGAGUGAGGUCU AGGGCACC | 1723 |
| 875 | GCCCUGCA G CCUCCCGC | 698 | GCGGGAGG GCCGAAAGGCGAGUGAGGUCU UGCACGGC | 1724 |
| 882 | AGCCUCCC G CAACGCCU | 699 | AGGCGUUG GCCGAAAGGCGAGUGAGGUCU GGGAGGCU | 1725 |
| 887 | CCCGCAAC G CCUGGCUG | 700 | CAGCCAGG GCCGAAAGGCGAGUGAGGUCU GUUGCGGG | 1726 |
| 892 | AACGCCUG G CUGGCCGU | 701 | ACGGCCAG GCCGAAAGGCGAGUGAGGUCU CAGGCGUU | 1727 |
| 896 | CCUGGCUG G CCGUGACC | 702 | GGUCACGG GCCGAAAGGCGAGUGAGGUCU CAGCCAGG | 1728 |
| 899 | GGCUGGCC G UGACCUCA | 703 | UGAGGUCA GCCGAAAGGCGAGUGAGGUCU GGCCAGCC | 1729 |
| 511 | CCUCAAAC G CCUAGCUG | 704 | CAGCUAGG GCCGAAAGGCGAGUGAGGUCU GUUUGAGG | 1730 |
| 916 | AACGCCUA G CUGCCAAU | 705 | AUUGGCAG GCCGAAAGGCGAGUGAGGUCU UAGGCGUU | 1731 |
| 919 | GCCUAGCU G CCAAUGAC | 706 | GUCAUUGG GCCGAAAGGCGAGUGAGGUCU AGCUAGGC | 1732 |
| 930 | AAUGACCU G CAGGOCUG | 707 | CAGCCCUG GCCGAAAGGCGAGUGAGGUCU AGGUCAUG | 1733 |
| 935 | CCUGCAGG G CUGCGCUG | 708 | CAGCGCAG GCCGAAAGGCGAGUGAGGUCU CCUGCAGG | 1734 |
| 938 | GCAGGGCU G CGCUGUGG | 709 | CCACAGCG GCCGAAAGGCGAGUGAGGUCU AGCCCUGC | 1735 |
| 940 | AGGGCUGC G CUGUGGCC | 710 | GGCCACAG GCCGAAAGGCGAGUGAGGUCU GCAGCCCU | 1736 |
| 943 | GCUGCGCU G UGGCCACC | 711 | GGUGGCCA GCCGAAAGGCGAGUGAGGUCU AGCGCAGC | 1737 |
| 946 | GCGCUGUG G CCACCGGC | 712 | GCCGGUGG GCCGAAAGGCGAGUGAGGUCU CACAGCGC | 1738 |
| 953 | GGCCACCG G CCCUUACC | 713 | GGUAACGG GCCGAAAGGCCAGUGACGUCU CCGUCCCC | 1739 |
| 977 | CUGGACCG G CAGGGCCA | 714 | UGGCCCUG GCCGAAAGGCGAGUGAGGUCU COGUCCAG | 1740 |
| 982 | CCGGCAGG G CCACCGAU | 715 | AUCGGUGG GCCGAAAGGCGAGUGAGGUCU CCUGCCGG | 1741 |
| 996 | GAUGAGGA G CCGCUGGG | 716 | CCCAGCGG GCCGAAAGGCGAGUGAGGUCU UCCUCAUC | 1742 |
| 999 | GAGGAGCC G CUGGGGCU | 717 | AGCCCCAG GCCGAAAGGCGAGUGAGGUCU GGCUCCUC | 1743 |
| 1005 | CCGCUGGG G CUUCCCAA | 718 | UUGGGAAG GCCGAAAGCCGAGUGAGGUCU CCCAGCGG | 1744 |
| 1014 | CUUCCCAA G UGCUCCCA | 719 | UGGCAGCA GCCGAAAGGCGAGUGAGGUCU UUGGCAAG | 1745 |
| 1016 | UCCCAAGU G CUGCCAGC | 720 | GCUGGCAG GCCGAAAGGCGAGUGAGGUCU ACUUGGGA | 1746 |
| 1019 | CAAGUGCU G CCAGCCAG | 721 | CUGGCUGG GCCGAAAGGCGAGUGAGGUCU AGCACUUG | 1747 |
| 1023 | UGCUGCCA G CCAGAUCC | 722 | GCAUCUGG GCCGAAAGGCGAGUGAGGUCU UCOCACCA | 1748 |
| 1030 | AGCCAGAU G CCGCUGAC | 723 | GUCAGCGG GCCGAAAGGCGAGUGAG0UCU AUCUGGCU | 1749 |
| 1033 | CAGAUCCC G CUGACAAG | 724 | CUGGUCAG GCCGAAAGGCGAGUGAGGUCU COCAUCUG | 1750 |
| 1042 | CUGACAAG G CCUCACUA | 725 | UACUCACG GCCGAAAGGCGAGUGAGGUCU CUGGUCAG | 1751 |
| 1048 | ACOCCUCA G UACUCGAG | 726 | CUCCACUA GCCGAAAGGCGAGUGAGGUCU UCACGCCU | 1752 |
| 1056 | GUACUGGA G CCUGGAAC | 727 | CUUCCACC GCCGAAAGGCGAGUGACGUCU UCCAGUAC | 1753 |
| 1069 | GAAGACCA G CUUCGGCA | 728 | UGCCGAAG GCCGAAAGGCGAGUGAGGUCU UGGUCUUC | 1754 |
| 1075 | CAGCUUCG G CAGGCAAU | 729 | AUUGCCUG GCCGAAAGGCGAGUGAGGUCU CGAAGCUG | 1755 |
| 1079 | UUCGCCAG G CAAUGCGC | 730 | GCGCAUUG GCCCAAAGGCGAGUGAGGUCU CUGCCGAA | 1756 |
| 1084 | CAGGCAAU G CGCUGAAG | 731 | CUUCAGCG GCCGAAAGGCGAGUGAGGUCU AUUGCCUG | 1757 |
| 1086 | CGCAAUGC G CUGAAGGG | 732 | CCCUUCAC GCCGAAAGGCGAGUGAGGUCU GCAUUGCC | 1758 |
| 1097 | GAACCGAC G CCUCCCGC | 733 | GCGGCACG GCCGAAAGGCGAGUGAGGUCU GUCCCUUC | 1759 |
| 1099 | AGGGACGC G UCCCCCCC | 734 | GOCCOOCA GCCGAAAGGCCAGUGAGGUCU CCGUCCCU | 1760 |
| 1101 | CGACGCGU G CCGCCCGG | 735 | CCGGGCGG GCCGAAAGGCGAGUGAGGUCU ACGCGUCC | 1761 |
| 1104 | CCCCUCCC G CCCGGUGA | 736 | UCACCGGG GCCGAAAGGCGAGUGAGGUCU GGCACGCG | 1762 |
| 1109 | GCCCCCCC G UCACACCC | 737 | GOCUCUCA GCCGAAAGGCGAGUCAGGUCU CGGGCGGC | 1763 |
| 1115 | CGGUGACA G CCCGCCGG | 738 | CCGGCGGG GCCGAAAGGCGAGUGAGGUCU UGUCACCG | 1764 |
| 1119 | CACACCCC G CCGGGCAA | 739 | UUGCCCGG GCCGAAAGGCGAGUGAGGUCU GGGCUGUC | 1765 |
| 1124 | CCGCCCGG G CAACGGCU | 740 | AGCCGUUG GCCGAAAGGCGAGUGAGGUCU CCCGCGCG | 1766 |
| 1130 | GCGCAACG G CUCUGCCC | 741 | GGCCAGAG GCCCAAAGGCGAGUGAGGUCU CGUUGCCC | 1767 |
| 1136 | COCCUCUG G CCCACGGC | 742 | GCCGUGGG GCCGAAAGGCGAGUGAGGUCU CAGAGCCG | 1768 |
| 1143 | CGCCCACC G CACAUCAA | 743 | UUGAUCUG GCCGAAAGGCGAGUGAGGUCU CCUCCGCC | 1769 |
| 1173 | CCCACUCU G CCUCGCUC | 744 | GACCCAGG GCCGAAAGGCGAGUCAGGUCU AGAGUCCC | 1770 |
| 1178 | UCUGCCUG G CUCUGCUG | 745 | CAGCAGAG GCCGAAAGGCGAGUGAGGUCU CAGGCAGA | 1771 |
| 1182 | CUGGCUCU G CUGAGCCC | 746 | GGGCUCAG GCCGAAAGGCGAGUGAGGUCU AGAGCCAG | 1772 |
| 1188 | UCUGCUGA G CCCCCGCU | 747 | AGCGGGGG GCCGAAAGGCGAGUGAGGUCU UCAGCAGA | 1773 |
| 1194 | CAGCCCCC G CUCACUGC | 748 | GCAGUGAG GCCGAAAGGCGAGUGAGGUCU GGGGGCUC | 1774 |
| 1201 | CGCUCACU G CAGUGCGG | 749 | CCGCACUG GCCGAAAGGCGAGUGAGGUCU AGUGAGCG | 1775 |
| 1204 | UCACUGCA G UGCGGCCC | 750 | GGGCCGCA GCCGAAAGGCGAGUGAGGUCU UGCAGUGA | 1776 |
| 1206 | ACUGCAGU G CGGCCCGA | 751 | UCGGGCCG GCCGAAAGGCGAGUGAGGUCU ACUGCAGU | 1777 |
| 1209 | GCAGUGCG G CCCGAGGG | 752 | CCCUCGGG GCCGAAAGGCGAGUGAGGUCU CGCACUGC | 1778 |
| 1217 | GCCCGAGG G CUCCGAGC | 753 | GCUCGGAG GCCGAAAGGCGAGUGAGGUCU CCUCGGGC | 1779 |
| 1224 | GGCUCCGA G CCACCAGG | 754 | CCUGGUGG GCCGAAAGGCGAGUGAGGUCU UCCGAGCC | 1780 |
| 1233 | CCACCAGG G UUCCCCAC | 755 | GUGGGGAA GCCGAAAGGCGAGUGAGGUCU CCUGGUGG | 1781 |
| 1247 | CACCUCGG G CCCUCGCC | 756 | GGCGAGGG GCCGAAAGGCGAGUGAGGUCU CCGAGGUG | 1782 |
| 1253 | GGGCCCUC G CCGGAGGC | 757 | GCCUCCGG GCCGAAAGGCGAGUGAGGUCU GAGGGCCC | 1783 |
| 1260 | CGCCGGAG G CCAGGCUG | 758 | CAGCCUGG GCCGAAAGGCGAGUGAGGUCU CUCCGGCG | 1784 |
| 1265 | GAGOCCAG G CUGUUCAC | 759 | GUGAACAG GCCGAAAGGCGAGUGAGGUCU CUGGCCUC | 1785 |
| 1268 | GCCAGGCU G UUCACGCA | 760 | UGCGUGAA GCCGAAAGGCGAGUGAGGUCU AGCCUGGC | 1786 |
| 1274 | CUGUUCAC G CAAGAACC | 761 | GGUUCUUG GCCGAAAGGCGAGUGAGGUCU GUGAACAG | 1787 |
| 1253 | CAAGAACC G CACCCGCA | 762 | UGCGGGUG GCCGAAAGGCGAGUGAGGUCU GGUUCUUG | 1788 |
| 1289 | CCGCACCC G CAGCCACU | 763 | AGUGOCUG GCCGAAAGGCGAGUGAGGUCU GGGUGCGG | 1789 |
| 1292 | CACCCGCA G CCACUGCC | 764 | GGCAGUGG GCCGAAAGGCGAGUGAGGUCU UGCGGGUG | 1790 |
| 1298 | CAGCCACU G CCGUCUGG | 765 | CCAGACGG GCCGAAAGGCGAGUGAGGUCU AGUGGCUG | 1791 |
| 1301 | CCACUGCC C UCUGGGCC | 766 | GGCCCAGA GCCGAAAGGCGAGUGAGGUCU GGCAGUGG | 1792 |
| 1307 | CCGUCUGG G CCAGGCAG | 767 | CUGCCUGG GCCGAAAGGCGAGUGAGGUCU CCAGACGG | 1793 |
| 1312 | UGGOCCAG G CAGGCAGC | 768 | GCUGCCUG GCCGAAAGGCGAGUGAGGUCU CUGGCCCA | 1794 |
| 1316 | CCAGGCAG G CAGCGGGG | 769 | CCCCGCUG GCCGAAAGGCGAGUGAGGUCU CUGCCUGG | 1795 |
| 1319 | GGCAGGCA G CGGGCGUG | 770 | CACCCCCG GCCCAAAGGCGACUCAGGUCU UGCCUGCC | 1796 |
| 1325 | CAGCGGGG G UGCCGGGA | 771 | UCCCGCCA GCCGAAAGCCGAGUCAGGUCU CCCCGCUG | 1797 |
| 1328 | COGOGGUG G CGGGACUG | 772 | CAGUCCCG GCCGAAAGGCGAGUGAGGUCU CACCCCCG | 1798 |
| 1337 | CGGGACUG G UGACUCAC | 773 | CUCAGUCA GCCGAAACGCGAGUGAGGUCU CAGUCCCG | 1799 |
| 1349 | CUCAGAAG G CUCAGGUG | 774 | CACCUGAG GCCGAAAGGCGAGUCAGGUCU CUUCUGAG | 1800 |
| 1355 | AGOCUCAG G UGCCCUAC | 775 | GUAGGOCA GCCGAAAGGCGACUGAGGUCU CUGAGCCU | 1801 |
| 1357 | GCUCAGGU G CCCUACCC | 776 | GGGUAGGG GCCGAAAGGCGAGUGAGGUCU ACCUGAGC | 1802 |
| 1367 | CCUACCCA G CCUCACCU | 777 | AGGUGAGG GCCGAAACGCGAGUGAGCUCU UGCGUACG | 1803 |
| 1376 | CCUCACCU G CAGCCUCA | 778 | UGAGGCUG GCCGAAAGGCGAGUGAGGUCU AGGUGAGG | 1804 |
| 1379 | CACCUCCA G CCUCACCC | 779 | GGGUGACG GCCGAAAGCCGAGUGAGGUCU UCCAGGUG | 1805 |
| 1394 | CCCCCUGG G CCUGGCGC | 780 | GCGCCAGG GCCGAAAGGCGAGUGAGGUCU CCAGGGGG | 1806 |
| 1399 | UCGCCCUC G CGCUGCUC | 781 | CACCAGCG GCCGAAAGCCGAGUGAGGUCU CAGCCCCA | 1807 |
| 1401 | CGCCUGGC G CUGGUGCU | 782 | ACCACCAG GCCGAAAGGCGAGUGACGUCU CCCAGGCC | 1808 |
| 1405 | UGGCGCUG G UGCUGUGG | 783 | CCACAGCA GCCGAAAGGCGAGUGAGGUCU CAGCGCCA | 1809 |
| 1407 | GCGCUGGU G CUGUGGAC | 784 | CUCCACAG GCCGAAAGGCGAGUGAGGUCU ACCAGCGC | 1810 |
| 1410 | CUGGUGCU G UGGACAGU | 785 | ACUCUCCA GCCGAAAGGCGAGUGAGGUCU AGCACCAG | 1811 |
| 1417 | UGUGGACA G UGCUUGGC | 786 | CCCAAGCA GCCGAAAGGCGAGUGAGGUCU UCUCCACA | 1812 |
| 1419 | UCGACAGU G CUUGGGCC | 787 | GGCCCAAG GCCGAAAGGCGAGUGAGGUCU ACUGUCCA | 1813 |
| 1425 | GUGCUUGG G CCCUCCUC | 788 | CAGCAGGG GCCGAAAGGCGAGUGAGGUCU CCAAGCAC | 1814 |
| 1430 | UGGGCCCU G CUGACCCC | 789 | GGGGUCAG GCCCAAAGGCGAGUGAGGUCU AGGGCCCA | 1815 |

Input Sequence = AF283463. Cut Site = G/Y
Arm Length = S. Core Sequence = GCcgaaagGCGaGuCaaGGuCu
AF283463 (Homo sapiens Nogo receptor mRNA, complete cds.; 1441 bp)

TABLE VI
Human NOGO Receptor DNAzyme and Substrate Sequence

| | | | | |
|---|---|---|---|---|
| | | Seq | | |
| Pos | Substrate | ID | DNAzyme | Seq ID |

| | | | | |
|---|---|---|---|---|
| 10 | CAACCCCU A CGAUGAAG | 1 | CTTCATCG GGCTAGCTACAACGA AGGGGTTG | 1816 |
| 108 | GCCUGCGU A UGCUACAA | 3 | TTGTAGCA GGCTAGCTACAACGA ACGCAGGC | 1817 |
| 113 | CGUAUGCU A CAAUGAGC | 4 | GCTCATTG GGCTAGCTACAACGA AGCATACG | 1818 |
| 408 | GGCCGCCU A CACACGCU | 26 | AGCGTCTG GGCTAGCTACAACGA AGGCGGCC | 1819 |
| 485 | CCUGCAGU A CCUCUACC | 29 | CGTAGAGG GGCTAGCTACAACGA ACTGCAGG | 1820 |
| 491 | GUACCUCU A CCUGCAGG | 31 | CCTGCAGG GGCTAGCTACAACGA AGAGGTAC | 1821 |
| 636 | CGUCUCCU A CUGCACCA | 45 | TGGTGCAG GGCTAGCTACAACGA AGGAGACG | 1822 |
| 707 | GACACUCU A UCUGUUUG | 51 | CAAACAGA GGCTAGCTACAACGA ACAGTGTC | 1823 |
| 726 | AACAAUCU A UCAGCGCU | 56 | AGCGCTGA GGCTAGCTACAACGA AGATTGTT | 1824 |
| 773 | CCUGCAGU A CCUGAGGC | 58 | GCCTCAGG GGCTAGCTACAACGA ACTGCACG | 1825 |
| 959 | CGGCCCUU A CCAUCCCA | 70 | TGGGATGG GGCTAGCTACAACGA AAGGGCCG | 1826 |
| 1050 | GCCUCAGU A CUGGAGCC | 76 | GGCTCCAG GGCTAGCTACAACGA ACTGAGGC | 1827 |
| 1362 | GGUGCCCU A CCCAGCCU | 97 | AGGCTGGG GGCTAGCTACAACGA AGGGCACC | 1828 |
| 51 | CUGCUGGC A UGGGUGCU | 107 | AGCACCCA GGCTAGCTACAACGA GCCAGCAG | 1829 |
| 90 | GCAGCCCC A UGCCCAGG | 118 | CCTGGGCA GGCTAGCTACAACGA GGGGCTGC | 1830 |
| 175 | CCGUGGGC A UCCCUGCU | 143 | AGCAGGGA GGCTAGCTACAACGA GCCCACGG | 1831 |
| 196 | GCCAGCGC A UCUUCCUG | 152 | CAGGAAGA GGCTAGCTACAACGA GCGCTGGC | 1832 |
| 206 | CUUCCUGC A CGGCAACC | 156 | GGTTGCCG GGCTAGCTACAACGA GCAGGAAG | 1833 |
| 569 | CUUCCUGC A CGGCAACC | 156 | GGTTGCCG GGCTAGCTACAACGA GCAGGAAG | 1834 |
| 217 | GCAACCGC A UCUCGCAU | 159 | ATGCGAGA GGCTAGCTACAACGA GCGGTTGC | 1835 |
| 224 | CAUCUCGC A UGUOCCAG | 161 | CTGGCACA GGCTAGCTACAACGA GCGAGATG | 1836 |
| 262 | GCAACCUC A CCAUCCUG | 175 | CAGGATGG GGCTAGCTACAACGA GAGGTTGC | 1837 |
| 265 | ACCUCACC A UCCUGUOG | 177 | CCACAGGA GGCTAGCTACAACGA GGTGAGGT | 1838 |
| 278 | GUGGCUGC A CUCGAAUG | 181 | CATTCGAG GGCTAGCTACAACGA GCAGCCAC | 1839 |
| 316 | CUGCCUUC A CUGGCCUG | 189 | CAGGCCAG GGCTAGCTACAACGA GAAGGCAG | 1840 |
| 360 | GAUAAUGC A CAGCUCCG | 203 | CGGAGCTG GGCTAGCTACAACGA GCATTATC | 1841 |
| 385 | ACCCUGCC A CAUUCCAC | 212 | GTGGAATG GGCTAGCTACAACGA GGCAGGGT | 1842 |
| 387 | CCUGCCAC A UUCCACGG | 213 | CCGTGGAA GGCTAGCTACAACCA GTGGCAGG | 1843 |
| 392 | CACAUUCC A CGGCCUGG | 215 | CCAGGCCG GGCTAGCTACAACGA GGAATGTG | 1844 |
| 410 | CCGCCUAC A CACGCUGC | 221 | GCAGCGTG GGCTAGCTACAACGA GTAGGCGG | 1849 |
| 412 | GCCUACAC A CGCUGCAC | 222 | GTGCAGCG GGCTAGCTACAACGA GTGTAGGC | 1846 |
| 419 | CACGCUGC A CCUGGACC | 224 | GGTCCAGG GGCTAGCTACAACGA GCAGCGTG | 1847 |
| 516 | CUGCAGGC A CUGCCUGA | 253 | TCAGGCAG GGCTAGCTACAACGA GCCTGCAG | 1848 |
| 529 | CUGAUGAC A CCUUCCGC | 257 | GCGGAAGG GGCTAGCTACAACGA GTCATCAG | 1849 |
| 553 | GCAACCUC A CACACCUC | 266 | GAGGTGTG GGCTAGCTACAACGA GAGGTTGC | 1850 |
| 555 | AACCUCAC A CACCUCUU | 267 | AAGAGGTG GGCTAGCTACAACGA GTGAGGTT | 1851 |
| 557 | CCUCACAC A CCUCUUCC | 268 | GGAAGAGG GGCTAGCTACAACGA GTGTGAGG | 1852 |
| 580 | GCAACCGC A UCUCCAGC | 275 | GCTGGAGA GGCTAGCTACAACGA GCGGTTGC | 1853 |
| 617 | UGGGCUGC A CAGCCUCG | 285 | CGAGGCTG GGCTAGCTACAACGA GCAGCCCA | 1854 |
| 641 | CCUACUGC A CCAGAACC | 294 | GGTTCTGG GGCTAGCTACAACGA GCAGTAGG | 1855 |
| 659 | CGUGGCCC A UGUGCACC | 300 | GGTGCACA GGCTAGCTACAACGA GGGCCACG | 1856 |
| 665 | CCAUGUGC A CCCGCAUG | 301 | CATGCGGG GGCTAGCTACAACGA GCACATGG | 1857 |
| 671 | GCACCCGC A UGCCUUCC | 304 | GGAAGGCA GGCTAGCTACAACGA GCGGGTGC | 1858 |
| 697 | GCCGCCUC A UGACACUC | 313 | GAGTGTCA GGCTAGCTACAACGA GAGGCGGC | 1859 |
| 702 | CUCAUGAC A CUCUAUCU | 314 | AGATAGAG GGCTAGCTACAACGA GTCATGAG | 1860 |
| 739 | CGCUGCCC A CUGAGGCC | 326 | GGCCTCAG GGCTAGCTACAACGA GGGCAGCG | 1861 |
| 816 | UGCCGGGC A CGCCCACU | 352 | AGTGGGCG GGCTAGCTACAACGA GCCCGGCA | 1862 |
| 822 | GCACGCCC A CUCUGGGC | 353 | GCCCAGAG GGCTAGCTACAACGA GGGCGTGC | 1863 |
| 949 | CUGUGGCC A CCGGCCCU | 396 | AGGGCCGG GGCTAGCTACAACGA GGCCACAG | 1864 |
| 962 | CCCUUACC A UCCCAUCU | 402 | AGATGGGA GGCTAGCTACAACGA GGTAAGGG | 1865 |
| 967 | ACCAUCCC A UCUGGACC | 405 | GGTCCAGA GGCTAGCTACAACGA GGGATGGT | 1866 |
| 985 | GCAGGGCC A CCGAUGAG | 410 | CTCATCGG GGCTAGCTACAACGA GGCCCTGC | 1867 |
| 1140 | UCUGGCCC A CGGCACAU | 450 | ATGTGCCG GGCTAGCTACAACGA GGGCCAGA | 1868 |
| 1145 | CCCACCGC A CAUCAAUG | 451 | CATTGATG GGCTAGCTACAACGA GCCGTGGG | 1869 |
| 1147 | CACGGCAC A UCAAUGAC | 452 | GTCATTGA GGCTAGCTACAACGA GTGCCGTG | 1870 |
| 1158 | AAUGACUC A CCCUUUGG | 455 | CCAAAGGG GGCTAGCTACAACGA GAGTCATT | 1871 |
| 1198 | CCCCGCUC A CUGCAGUG | 471 | CACTUCAG GGCTAGCTACAACGA GAGCGGGG | 1872 |
| 1227 | UCCGAGCC A CCAGGGUU | 479 | AACCCTGG GGCTAGCTACAACGA GGCTCGGA | 1873 |
| 1240 | GGUUCCCC A CCUCGGGC | 485 | GCCCGAGG GGCTAGCTACAACGA GGGGAACC | 1874 |
| 1272 | GGCUGUUC A CGCAAGAA | 495 | TTCTTGCG GGCTAGCTACAACGA GAACAGCC | 1875 |
| 1285 | AGAACCGC A CCCGCAGC | 498 | GCTGCGGG GGCTAGCTACAACGA GCGGTTCT | 1876 |
| 1295 | CCGCAGCC A CUGCCGUC | 503 | GACQOCAG GGCTAGCTACAACGA GGCTGCGG | 1877 |
| 1372 | CCAGCCUC A CCUGCAGC | 524 | GCTGCAGG GGCTAGCTACAACGA GAGGCTGG | 1878 |
| 1384 | GCAGCCUC A CCCCCCUG | 530 | CAGGGGGG GGCTAGCTACAACGA GAGGCTGC | 1879 |
| 22 | UGAAGAGG G CGUCCGCU | 547 | AGCGGACG GGCTAGCTACAACGA CCTCTTCA | 1880 |
| 24 | AAGAGGGC G UCCGCUGG | 548 | CCAGCGGA GGCTAGCTACAACGA GCCCTCTT | 1881 |
| 28 | GGGCGUCC G CUGGAGGG | 549 | CCCTCCAG GGCTAGCTACAACGA GGACGCCC | 1882 |
| 38 | UGGAGGGA G CCGGCUGC | 550 | GCAGCCGG GGCTAGCTACAACGA TCCCTCCA | 1883 |
| 42 | GGGAGCCG G CUGCUGGC | 551 | GCCAGCAG GGCTAGCTACAACGA CGGCTCCC | 1884 |
| 45 | AGCCGGCU G CUGGCAUG | 552 | CATGCCAG GGCTAGCTACAACGA AGCCGGCT | 1885 |
| 49 | GGCUGCUG G CAUGGGUG | 553 | CACCCATG GGCTAGCTACAACGA CAGCAGCC | 1886 |
| 55 | UGGCAUGG G UGCUGUGG | 554 | CCACAGCA GGCTAGCTACAACGA CCATGCCA | 1887 |
| 57 | GCAUGGGU G CUGUGGCU | 555 | AGCCACAG GGCTAGCTACAACGA ACCCATGC | 1888 |
| 60 | UGGGUGCU G UGGCUGCA | 556 | TGCAGCCA GGCTAGCTACAACGA AGCACCCA | 1889 |
| 63 | GUCCUGUG G CUGCAGGC | 557 | GCCTGCAG GGCTAGCTACAACGA CACAGCAC | 1890 |
| 66 | CUGUGGCU G CAGGCCUG | 558 | CAGGCCTG GGCTAGCTACAACGA AGCCACAG | 1891 |
| 70 | GGCUGCAG G CCUGGCAG | 559 | CTGCCAGG GGCTAGCTACAACGA CTGCAGCC | 1892 |
| 75 | CAGGCCUG G CAGGUGGC | 560 | GCCACCTG GGCTAGCTACAACGA CAGGCCTG | 1893 |
| 79 | CCUGGCAG G UGGCAGCC | 561 | GGCTGCCA GGCTAGCTACAACGA CTGCCAGG | 1894 |
| 82 | GGCAGGUG G CAGCCCCA | 562 | TGGGGCTG GGCTAGCTACAACGA CACCTGCC | 1895 |
| 85 | AGGUGGCA G CCCCAUGC | 563 | GCATGGGG GGCTAGCTACAACGA TGCCACCT | 1896 |
| 92 | AGCCCCAU G CCCAGGUG | 564 | CACCTGGG GGCTAGCTACAACGA ATGGGGCT | 1897 |
| 98 | AUGCCCAG G UGCCUGCG | 565 | CGCAGGCA GGCTAGCTACAACGA CTGGGCAT | 1898 |
| 100 | GCCCAGGU G CCUGCGUA | 566 | TACGCAGG GGCTAGCTACAACGA ACCTGGGC | 1899 |
| 104 | AGGUGCCU G CGUAUGCU | 567 | AGCATACG GGCTAGCTACAACGA AGGCACCT | 1900 |
| 106 | GUGCCUGC G UAUGCUAC | 568 | GTAGCATA GGCTAGCTACAACGA GCAGGCAC | 1901 |
| 110 | CUGCGUAU G CUACAAUG | 569 | CATTGTAG GGCTAGCTACAACGA ATACGCAG | 1902 |
| 120 | UACAAUGA G CCCAAGGU | 570 | ACCTTGGG GGCTAGCTACAACGA TCATTGTA | 1903 |
| 127 | AGCCCAAG G UGACGACA | 571 | TGTCGTCA GGCTAGCTACAACGA CTTGGGCT | 1904 |
| 137 | GACGACAA G CUGCCCCC | 572 | GGGGGCAG GGCTAGCTACAACGA TTGTCGTC | 1905 |
| 140 | GACAAGCU G CCCCCAGC | 573 | GCTGGGGG GGCTAGCTACAACGA AGCTTGTC | 1906 |
| 147 | UGCCCCCA G CAGGGCCU | 574 | AGGCCCTG GGCTAGCTACAACGA TGGGGGCA | 1907 |
| 152 | CCAGCAGG G CCUGCAGG | 575 | CCTGCAGG GGCTAGCTACAACGA CCTGCTGG | 1908 |
| 156 | CAGGGCCU G CAGGCUGU | 576 | ACAGCCTG GGCTAGCTACAACGA AGGCCCTG | 1909 |
| 160 | GCCUGCAG G CUGUGCCC | 577 | GGGCACAG GGCTAGCTACAACGA CTGCAGGC | 1910 |
| 163 | UGCAGGCU G UGCCCUUG | 578 | CACGGGCA GGCTAGCTACAACGA AGCCTGCA | 1911 |
| 165 | CAGGCUGU G CCCGUGGG | 579 | CCCACGGG GGCTAGCTACAACGA ACAGCCTG | 1912 |
| 169 | CUGUGCCC G UGGGCAUC | 580 | GATGCCCA GGCTAGCTACAACGA GGGCACAG | 1913 |
| 173 | GCCCGUGG G CAUCCCUG | 581 | CACCGATG GGCTAGCTAOAACGA CCACGGGC | 1914 |
| 181 | GCAUCCCU C CUCCCACC | 582 | GCTCGCAG CCCTAGCTACAACGA AGGGATGC | 1915 |
| 184 | UCCCUGCU G CCAGCCAG | 583 | CTGGCTGG GGCTAGCTACAACGA AGCAGGGA | 1916 |
| 188 | UCCUGCCA G CCAGCGCA | 584 | TGCGCTGG GGCTAGCTACAACGA TGGCAGCA | 1917 |
| 192 | GCCACCCA G CGCAUCUU | 585 | AACATGCG GGCTAGCTACAACGA TGCCTCCC | 1918 |
| 194 | CACCCAGC G CAUCUUCC | 586 | GGAACATG GGCTAGCTACAACGA GCTGGCTG | 1919 |
| 204 | AUCUUCCU G CACGGCAA | 587 | TTGCCCTG GGCTACCTACAACGA ACGAACAT | 1920 |
| 209 | CCUCCACC G CAACCCCA | 588 | TGCCCTTC GGCTAGCTACAACGA CGTCCAGC | 1921 |
| 572 | CCUCCACC C CAACCCCA | 588 | TGCGGTTC GGCTAGCTACAACGA CCTCCACC | 1922 |
| 215 | CGGCAACC G CAUCUCGC | 589 | GCGAGATG GGCTAGCTACAACGA GGTTGCCG | 1923 |
| 222 | CGCAUCUC G CAUGUCCC | 590 | GGCACATG GGCTAGCTACAACGA GAGATGCG | 1924 |
| 226 | UCUCGCAU G UGCCAGCU | 591 | AGCTCCCA GGCTAGCTACAACGA ATCCCACA | 1925 |
| 228 | UCCCAUGU G CCACCUCC | 592 | CCAGCTCG GGCTACCTACAACGA ACATCCCA | 1926 |
| 232 | AUCUGCCA G CUCCCACC | 593 | GCTCCCAG GCCTACCTACAACCA TCCCACAT | 1927 |
| 235 | UGCCACCU C CCAGCUUC | 594 | GAAGCTGG GGCTAGCTACAACGA AGCTGGCA | 1928 |
| 239 | ACCUOCCA C CUUCCGUC | 595 | CACGGAAC GGCTAGCTACAACCA TGGCACCT | 1929 |
| 245 | CACCUUCC C UCCCUGCC | 596 | UCCAGOCA GGCTAGCTACAACGA GGAACCTC | 1930 |
| 247 | CCUUCCCU C CCUGCCCC | 597 | CCCGCAGC GGCTAGCTACAACGA ACCCAACC | 1931 |
| 251 | CCGUCCCU G CCGCAACC | 598 | CCTTCCGG CGCTAGCTACAACGA AGCCACGC | 1932 |
| 254 | UCCCUGCC G CAACCUCA | 599 | TCAGGTTG GGCTAGCTACAACCA CGCAGGCA | 1933 |
| 270 | ACCAUCCU G UCCCUCCA | 600 | TGCACCCA GGCTAGCTACAACGA ACGATGGT | 1934 |
| 273 | AUCCUCUG G CUCCACUC | 601 | GAGTCCAG GGCTAGCTACAACGA CACACCAT | 1935 |
| 276 | CUCUGGCU C CACUCGAA | 602 | TTCCACTG CGCTAGCTACAACGA ACCCACAG | 1936 |
| 286 | ACUCGAAU C UGCUCGCC | 603 | GGCCAGCA GGCTAGCTACAACGA ATTCGACT | 1937 |
| 288 | UCCAAUGU C CUCGCCCC | 604 | CCGGCCAG GGCTAGCTACAACGA ACATTCCA | 1938 |
| 292 | AUCUCCUG C CCCGAAUU | 605 | AATTCGGG CGCTAGCTACAACGA CAGCACAT | 1939 |
| 304 | GAAUUGAU C CCCCUCCC | 606 | CCCACCCG GGCTAGCTACAACGA ATCAATTC | 1940 |
| 307 | UUCAUGCG G CUGCCUUC | 607 | CAAGCCAG CCCTACCTACAACGA CCCATCAA | 1941 |
| 310 | AUCCCCCU C CCUUCACU | 608 | AGTGTAAGG GGCTAGCTACAACCA AGCCGCAT | 1942 |
| 320 | CUUGACUC C CCUGGCCC | 609 | GGGCCAGG GGCTAGCTACAACCA CAGTCAAG | 1943 |
| 325 | CUCCCCUC G CCCUCCUC | 610 | CAGGACUC GGCTACCTACAACCA CAGCCCAC | 1944 |
| 336 | CUCCUCCA C CACCUCCA | 611 | TCCACCTC CGCTACCTACAACCA TCGACCAG | 1945 |
| 339 | CUCCACCA C CUGCACCU | 612 | ACCTCCAG GGCTAGCTACAACGA TGCTCCAG | 1946 |
| 350 | CCACCUCA C CCAUAAUC | 613 | CATTATCG GGCTAGCTACAACCA TCACGTCC | 1947 |
| 358 | CCCAUAAU C CAGACCUC | 614 | CACCTCTG GCCTACCTAGAACGA ATTATCCC | 1948 |
| 363 | AAUCCAGA C CUCCCCUC | 615 | GACCCCAG CGCTACCTACAACGA TCTCCATT | 1949 |
| 369 | CACCUCCC C UCUCUCCA | 616 | TCCACACA GGCTAGCTACAACCA CGGACCTG | 1950 |
| 373 | UCCGGUCU G UGGACCCU | 617 | AGGGTCCA GGCTAGCTAGCTACAACGA AGACCGGA | 1951 |
| 382 | UCCACCCU C CCACAUUC | 618 | GAATCTCG CGCTAGCTACAACGA AGGGTCCA | 1952 |
| 395 | AUUCCACC C CCUGGCCC | 619 | CCCCCAGG GCCTAGCTACAACCA CCTCCAAT | 1953 |
| 401 | CCCCCUCC C CCCCCUAC | 620 | CTACCCCC CGCTAGCTACAACCA CCACCCCC | 1954 |
| 404 | CCUCCCCC C CCUACACA | 621 | TGTGTAGG GGCTAGCTACAACCA CCCCCACC | 1955 |
| 414 | CUACACAC C CUCCACCU | 622 | ACCTGCAC GGCTAGCTACAACGA CTCTCFAC | 1956 |
| 417 | CACACGCU G CACCUGGA | 623 | TCCAGGTG GGCTAGCTACAACGA AGCGTGTG | 1957 |
| 428 | CCUCGACC C CUCCCCCC | 624 | GGCCGCAG GGCTACCTACAACGA CCTCGAGC | 1958 |
| 431 | CCACCCCU C CCGCCUCC | 625 | GCAGGCCG GGCTACCTACAACGA ACCCCTCC | 1959 |
| 434 | CCCCUCCC C CCUCCAGC | 626 | CCTGCAGG CCCTAGCTACAACCA CCCACCCC | 1960 |
| 438 | UCCCCCCU G CACCACCU | 627 | ACCTCCTC CGCTAGCTACAACCA ACCCCGCA | 1961 |
| 444 | CUCCACCA G CUGCCCCC | 628 | CCGCCCAG CCCTACCTACAACGA TCCTGCAC | 1962 |
| 449 | CCAGCUCC C CCCGCGCC | 629 | GCCCCGGC CCCTAGCTACAACGA CCAGCTCC | 1963 |
| 456 | GGCCCGGG G CUCUUCCC | 630 | CGGAACAG CCCTAGCTACAACCA CCCGGGCC | 1964 |
| 459 | CCGGGGCU G UUCCGCGG | 631 | CCGCGGAA GGCTAGCTACAACGA AGCCCCGG | 1965 |
| 464 | GCUGUUCC G CGGCCUGG | 632 | CCAGGCCG GGCTAGCTACAACGA GGAACAGC | 1966 |
| 467 | GUUCCGCG G CCUOGCUG | 633 | CAGCCAGO GGCTAGCTACAACGA CGCGGAAC | 1967 |
| 472 | GCOGCCUG G CUGCCCUG | 634 | CAGGOCAG GGCTAGCTACAACGA CAGGCCGC | 1968 |
| 475 | GCCUGGCU G CCCUGCAG | 635 | CTGCAGGG GGCTAGCTACAACGA AGCCAGGC | 1969 |
| 480 | GCUGCCCU G CAGUACCU | 636 | AGGTACTG CGCTAGCTACAACGA AGGGCAGC | 1970 |
| 483 | GCCCUGCA G UACCUCUA | 637 | TAGAGOTA GGCTAGCTACAACGA TGCAGGGC | 1971 |
| 495 | CUCUACCU G CAGGACAA | 638 | TTGTCCTG GGCTAGCTACAACGA AGGTAGAG | 1972 |
| 505 | AGGACAAC C CGCUGCAG | 639 | CTGCAGCG GGCTAGCTACAACGA GTTGTCCT | 1973 |
| 507 | GACAACGC G CUGCAGGC | 640 | GCCTGCAG GGCTAGCTACAACGA GCGTTGTC | 1974 |
| 510 | AACGCGCU G CAGGCACU | 641 | AGTGCCTG GGCTAGCTACAACGA AGCGCGTT | 1975 |
| 514 | CGCUGCAG G CACUGCCU | 642 | AGGCAGTG GGCTAGCTACAACGA CTGCAGCG | 1976 |
| 519 | CAGGCACU G CCUGAUGA | 643 | TCATCAGG GGCTAGCTACAACGA AGTGCCTG | 1977 |
| 536 | CACCUUCC G CGACCUGG | 644 | CCAGGTCG GGCTAGCTACAACGA GGAAGGTG | 1978 |
| 545 | CGACCUGG G CAACCUCA | 645 | TGAGGTTG GGCTAGCTACAACGA CCAGGTCG | 1979 |
| 667 | CUCUUCCU G CACGGCAA | 646 | TTGCCGTG GGCTAGCTACAACGA AGGAAGAG | 1980 |
| 578 | CGGCAACC C CAUCUCCA | 647 | TGGAGATG GGCTAGCTACAACGA GGTTGCCG | 1981 |
| 587 | CAUCUCCA G CGUGCCCG | 648 | CGGGCACG GGCTAGCTACAACGA TGGAGATG | 1982 |
| 589 | UCUCCAGC C UGCCCGAG | 649 | CTCGGGCA GGCTAGCTACAACGA GCTOGAGA | 1983 |
| 591 | UCCAGCGU G CCCGAGCG | 650 | CGCTCGGG GGCTAGCTACAACGA ACGCTGGA | 1984 |
| 597 | GUGCCCGA G CGCGCCUU | 651 | AAGGCGCG GGCTAGCTACAACGA TCGGGCAC | 1985 |
| 599 | GCCCGAGC G CGCCUUCC | 652 | GGAAGGCG GGCTAGCTACAACGA GCTCGGGC | 1986 |
| 601 | CCGAGCGC G CCUUCCGU | 653 | ACGGAAGG GGCTAGCTACAACGA GCGCTCGG | 1987 |
| 608 | CGCCUUCC C UGGGCUGC | 654 | GCAGCCCA GGCTAGCTACAACGA GGAAGGCG | 1988 |
| 612 | CUCCOUGO G CUCCACAG | 655 | CTGTGCAG GGCTAGCTACAACGA CCACGGAA | 1989 |
| 615 | CGUGGGCU G CACAGCCU | 656 | AGGCTGTG GGCTAGCTACAACGA AGCCCACG | 1990 |
| 620 | OCUOCACA G CCUCGACC | 657 | GGTCGAGG GGCTAGCTACAACGA TGTGCAGC | 1991 |
| 629 | CCUCGACC G UCUCCUAC | 658 | GTAGGAGA GGCTAGCTACAACGA GGTCGAGG | 1992 |
| 639 | CUCCUACU G CACCAGAA | 659 | TTCTGGTG GGCTAGCTACAACGA ACTAGGAG | 1993 |
| 650 | CCAGAACC C CGUGGCCC | 660 | GGGCCACG GGCTAGCTACAACGA GGTTCTGG | 1994 |
| 652 | AGAACCGC C UGGCCCAU | 661 | ATOGOCCA GGCTAGCTACAACGA GCGGTTCT | 1995 |
| 655 | ACCGCGUG G CCCAUGUG | 662 | CACATGGG GGCTAGCTACAACGA CACGCGGT | 1996 |
| 661 | UGGCCCAU C UGCACCCG | 663 | CGGGTGCA GGCTAGCTACAACGA ATGGGCCA | 1997 |
| 663 | CCCCAUGU C CACCCGCA | 664 | TGCGGGTG GGCTAGCTACAACCA ACATGGGC | 1998 |
| 669 | GUGCACCC C CAUGCCUU | 665 | AAGGCATG GGCTAGCTACAACGA GGGTGCAC | 1999 |
| 673 | ACCCGCAU G CCUUCCGU | 666 | ACGGAAGG GGCTAGCTACAACGA ATGCGGGT | 2000 |
| 680 | UGCCUUCC C UGACCUUG | 667 | CAAGGTCA GGCTAGCTACAACGA GGAAGGCA | 2001 |
| 689 | UGACCUUG C CCGCCUCA | 668 | TGAGGCGG GGCTAGCTACAACGA CAAGGTCA | 2002 |
| 692 | CCUUGGCC C CCUCAUGA | 669 | TCATGAGG GGCTAGCTACAACGA GGCCAAGG | 2003 |
| 711 | CUCUAUCU G UUUGCCAA | 670 | TTGGCAAA GGCTAGCTACAACGA AGATAGAG | 2004 |
| 715 | AUCUGUUU G CCAACAAU | 671 | ATTGTTGG GGCTAGCTACAACGA AAACAGAT | 2005 |
| 730 | AUCUAUCA C CGCUGCCC | 672 | GGGCACCG GGCTAGCTACAACGA TGATAGAT | 2006 |
| 732 | CUAUCAGC C CUGCCCAC | 673 | GTGGGCAG GGCTAGCTACAACGA GCTOATAG | 2007 |
| 735 | UCAGCGCU C CCCACUGA | 674 | TCAGTGGG GGCTAGCTACAACGA AGCGCTGA | 2008 |
| 745 | CCACUGAC C CCCUGGCC | 675 | GGCCAGGG GGCTAGCTACAACGA CTCAGTGG | 2009 |
| 751 | AGGCCCUG C CCCCCCUG | 676 | CAGGGGGG GGCTAGCTACAACGA CAGGGCCT | 2010 |
| 759 | CCCCCCCU C CCUCCCCU | 677 | AGGGCACG GGCTAGCTACAACGA AGGGGGGC | 2011 |
| 761 | CCCCCUCC C UGCCCUGC | 678 | GCAGGGCA GGCTAGCTACAACGA GCAGGGGG | 2012 |
| 763 | CCCUGCGU C CCCUGCAC | 679 | CTGCAGGG GGCTAGCTACAACGA ACOCAGGG | 2013 |
| 768 | CGUGCCCU C CAGUACCU | 680 | AGCTACTG GGCTAGCTACAACGA ACOCCACG | 2014 |
| 771 | CCCCUCCA C UACCUCAC | 681 | CTCAGCTA GGCTAGCTACAACGA TGCAGGGC | 2015 |
| 780 | UACCUGAG G CUCAACGA | 682 | TCGTTGAG GGCTAGCTACAACGA CTCAGGTA | 2016 |
| 799 | ACCCCUGG G UGUGUGAC | 683 | GTCACACA GGCTAGCTACAACGA CCAGGGGT | 2017 |
| 801 | CCCUGGGU C UGUGACUG | 684 | CAGTCACA GGCTAGCTACAACGA ACCCAGGG | 2018 |
| 803 | CUGGGUGU G UGACUGCC | 685 | GGCAGTCA GGCTAGCTACAACGA ACACCCAG | 2019 |
| 809 | GUGUGACU C CCGCGCAC | 686 | GTGCCCGG GGCTAGCTACAACGA AGTCACAC | 2020 |
| 814 | ACUGCCGG C CACGCCCA | 687 | TGGGCGTG GGCTAGCTACAACGA CCGGCAGT | 2021 |
| 818 | CCGGGCAC G CCCACUCU | 688 | AGAGTGGG GGCTAGCTACAACGA GTGCCCCG | 2022 |
| 829 | CACUCUGG C CCUGGCUG | 689 | CACCCACC GGCTAGCTACAACGA CCAGAGTG | 2023 |
| 834 | UGGCCCUG G CUCCAGAA | 690 | TTCTGCAG CGCTAGCTACAACCA CACGCCCA | 2024 |
| 837 | GCCUGCCU C CAGAAGUU | 691 | AACTTCTC CGCTAGCTACAACCA ACCCAGGC | 2025 |
| 843 | CUGCAGPA G UUCCCCCG | 692 | CCGCGGAA GGCTAGCTACAACGA TTCTGCAG | 2026 |
| 848 | GAAGUUCC G CCGCUCCU | 693 | AGGACCCC GGCTAGCTACAACCA CGAACTTC | 2027 |
| 851 | GUUCCGCC G CUCCUCCU | 694 | AGGAGGAC GGCTACCTACAACCA CGCGCAAC | 2028 |
| 865 | CCUCCCAG C UGCCCUGC | 695 | GCAGGGCA GCCTAGCTACAACGA CTCGCAGC | 2029 |
| 867 | UCCGAGGU C CCCUCCAC | 696 | CTCCACGG CCCTAGCTACAACCA ACCTCGCA | 2030 |
| 872 | GCUCCCCU G CAGCCUCC | 697 | CCACGCTG GGCTAGCTACAACGA AGGGCACC | 2031 |
| 875 | CCCCUGCA G CCUCCCCC | 698 | CCGGCACG CGCTACCTACAACGA TGCACCCC | 2032 |
| 882 | AGCCUCCC C CAACCCCU | 699 | AGGCGTTG CCCTAGCTACAACGA CCCACCCT | 2033 |
| 887 | CCCGCAAC C CCUGGCUC | 700 | CAGCCAGG GGCTACCTACAACGA GTTCCCCC | 2034 |
| 892 | AACCCCUG C CUCGCCCU | 701 | ACGGCCAG GGCTAGCTACAACGA CACGCCTT | 2035 |
| 896 | COUGUCUC G CCGUCACC | 702 | CGTCACCC CCCTACCTACAACGA CAGCCACC | 2036 |
| 899 | GCCUGGCC G UCACCUCA | 703 | TCACCTCA CGCTAGCTACAACCA GCCCACCC | 2037 |
| 911 | CCUCAAAC C CCUACCUC | 704 | CACCTACC CGCTAGCTACAACCA CTTTCACC | 2038 |
| 916 | AACGCCUA G CUCCCAAU | 705 | ATTGGCAG GCCTAGCTACAACGA TAGCCCTT | 2039 |
| 919 | GCCUACCU C CCAAUGAC | 706 | GTCATTGG CGCTAGCTACAACGA AGCTAGGC | 2040 |
| 930 | AAUGACCU G CACCCCUG | 707 | CAGCCCTG GGCTAGCTACAACCA AGGTCATT | 2041 |
| 935 | CCUCCACG C CUGCCCUC | 708 | CAGCGCAG CGCTAGCTACAACCA CCTGCAGC | 2042 |
| 938 | CCAGGGCU C CGCUGUCG | 709 | CCACAGCG GGCTACCTACAACGA ACCCCTGC | 2043 |
| 940 | AGGGCUGC G CUCUCCCC | 710 | CCCCACAG CCCTACCTACAACCA CCACCCCT | 2044 |
| 943 | GCUCCGCU G UGCCCACC | 711 | CCTCCCCA CGCTACCTACAACCA AGCCCACC | 2045 |
| 946 | GCGCUGUC C CCACCCCC | 712 | GCCGGTCG GGCTAGCTACAACGA CACAGCGC | 2046 |
| 953 | CCCCACCC C CCCUUACC | 713 | GGTAACCC CGCTAGCTACAACGA CGGTGGCC | 2047 |
| 977 | CUCCACOG C CACGGCCA | 714 | TCCCCCTG GGCTACCTACAACCA CCGTCCAC | 2048 |
| 982 | CCCGCACC C CCACCGAU | 715 | ATCGCTCC GCCTAGCTACAACCA CCTCCCCC | 2049 |
| 998 | CAUCACCA C CCCCUCCC | 716 | CCCACCCC CCCTACCTACAACCA TCCTCATC | 2050 |
| 999 | CACCACCC C CUCCCCCU | 717 | ACCCCCAC CCCTACCTACAACCA CCCTCCTC | 2051 |
| 1005 | CCCCUCCC C CUUCCCAA | 718 | TTGCGAAG CCCTACCTACAACCA CCCACCCC | 2052 |
| 1014 | CUUCCCAA C UCCUCCCA | 719 | TCCCACCA CCCTACCTACAACCA TTCCCAAC | 2053 |
| 1016 | UCCCAACU C CUCCCACC | 720 | CCTCCCAG CGCTAGCTACAACGA ACTTCGGA | 2054 |
| 1019 | CAAGUCCU C CCAGCCAC | 721 | CTGGCTGG GGCTACCTACAACGA AGCACTTC | 2055 |
| 1023 | UCCUCCCA C CCACAUCC | 722 | CCATCTCC CCCTACCTACAACGA TCCCACCA | 2056 |
| 1030 | ACCCACAU C CCCCUCAC | 723 | CTCACCGC GCCTAGCTACAACGA ATCTCCCT | 2057 |
| 1033 | CACAUCCC C CUCACAAG | 724 | CTTCTCAC GGCTAGCTACAACCA GCCATCTG | 2058 |
| 1042 | CUCACAAC C CCUCACUA | 725 | TACTCACG GGCTAGCTACAACCA CTTGTCAC | 2059 |
| 1048 | ACCCCUCA C UACUGGAC | 726 | CTCCAGTA GGCTAGCTACAACGA TGAGGCCT | 2060 |
| 1056 | GUACUCCA C CCUCCAAC | 727 | CTTCCAGG GGCTACCTACAACGA TCCACTAC | 2061 |
| 1069 | GAAGACCA C CUUCGCCA | 728 | TCCCCAAC CCCTACCTACAACCA TCCTCTTC | 2062 |
| 1075 | CAGCUUCC G CAGGCAAU | 729 | ATTGCCTG GCCTACCTACAACGA CCAACCTC | 2063 |
| 1079 | UUCCCCAC C CAAUCCCC | 730 | CCCCATTC CCCTACCTACAACCA CTCCCCAA | 2064 |
| 1084 | CACCCAAU C CCCUCAAC | 731 | CTTCACCG CCCTACCTACAACGA ATTCCCTC | 2065 |
| 1086 | CCCAAUGC C CUCAAGGG | 732 | CCCTTCAG CCCTACCTACAACCA CCATTCCC | 2066 |
| 1097 | GAAGGGAC G CGUGCCGC | 733 | GCGGCACG GGCTAGCTACAACGA GTCCCTTC | 2067 |
| 1099 | AGGGACGC G UGCCGCCC | 734 | GGGCGGCA GGCTAGCTACAACGA GCGTCCCT | 2068 |
| 1101 | GGACGCGU G CCGCCCGG | 735 | CCGGGCGG GGCTAGCTACAACGA ACGCGTCC | 2069 |
| 1104 | CGCGUGCC G CCCGGUGA | 736 | TCACCGGG GGCTAGCTACAACGA GGCACGCG | 2070 |
| 1109 | GCCGCCCG G UGACAGCC | 737 | GGCTGTCA GGCTAGCTACAACGA CGGGCGGC | 2071 |
| 1115 | CGGUGACA C CCCGCCGG | 738 | CCGGCCCG GGCTAGCTACAACGA TCTCACCC | 2072 |
| 1119 | GACAGCCC C CCGGGCAA | 739 | TTGCCCGG GCCTAGCTACAACGA GCGCTCTC | 2073 |
| 1124 | CCCCCCGG G CAACGGCU | 740 | AGCCGTTG GGCTAGCTACAACGA CCGGCGGG | 2074 |
| 1130 | GCGCAACG G CUCUGGCC | 741 | GGCCAGAG GGCTAGCTACAACGA CGTTGCCC | 2075 |
| 1136 | CGGCUCUG G CCCACGGC | 742 | GCCGTGGG GGCTAGCTACAACGA CAGAGCCG | 2076 |
| 1143 | GOCOCACO C CACAUCAA | 743 | TTGATGTG GCCTAGCTACAACCA CCTCCCCC | 2077 |
| 1173 | CCGACUCU C CCUGGCUC | 744 | GACCCACG GCCTACCTACAACCA ACACTCCC | 2078 |
| 1178 | UCUGCCUG G CUCUCCUC | 745 | CACCAGAG CCCTACCTACAACCA CACCCACA | 2079 |
| 1183 | CUGCCUCU G CUCAGCCC | 746 | CGGCTCAC CGCTAGCTACAACGA AGAGCCAG | 2080 |
| 1188 | UCUGCUCA G CCCCCCCU | 747 | AGCGGGGG GGCTAGCTACAACGA TCAGCAGA | 2081 |
| 1194 | GACCCCCC G CUCACUCC | 748 | GCAGTCAG GGCTAGCTACAACGA CCGCGCTC | 2082 |
| 1201 | CCCUCACU C CACUCCCG | 749 | CCGCACTC GCCTACCTACAACGA AGTCAGCG | 2083 |
| 1204 | UCACUCCA G UCCGGCCC | 750 | CCGCCGCA CGCTAGCTACAACGA TCCAGTGA | 2084 |
| 1206 | ACUCCACU G CGGCCCCA | 751 | TCCCGCCC GGCTAGCTACAACGA ACTCCAGT | 2085 |
| 1209 | GCACUCCG C CCCGAGCG | 752 | CCCTCGGC GCCTACCTACAACGA CCCACTGC | 2086 |
| 1217 | GCCCCAGC C CUCCCACC | 753 | CCTCCGAG GCCTACCTACAACGA CCTCGCGC | 2087 |
| 1224 | GGCUCCCA C CCACCACC | 754 | CCTGCTGG GCCTAGCTACAACGA TCGCACCC | 2088 |
| 1233 | CCACCAGC G UUCCCCAC | 755 | GTCGGCAA GGCTAGCTACAACGA CCTGGTGG | 2089 |
| 1247 | CACCUCGG G CCCUCGCC | 756 | GCCCAGGG CGCTACCTACAACCA CCCACGTC | 2090 |
| 1253 | CCGCCCUC G CCGCACCC | 757 | CCCTCCGG GCCTACCTACAACGA CAGCCCCC | 2091 |
| 1260 | CCCCCCAC C CCAGCCUC | 758 | CAGCCTGC CGCTACCTACAACGA CTCCCGCC | 2092 |
| 1265 | CAGCCCAC C CUCUUCAC | 759 | CTGAACAG CCCTAGCTACAACCA CTGGCCTC | 2093 |
| 1268 | GCCACGCU G UUCACGCA | 760 | TCCGTCAA GGCTAGCTACAACGA AGCCTGGC | 2094 |
| 1274 | CUCUCCAC G CAAGAACC | 761 | GGTTCTTG GGCTAGCTACAACGA GTGAACAC | 2095 |
| 1283 | CAACAACC C CACCCGCA | 762 | TGCGGCTG GCCTAGCTACAACGA GCTTCTTC | 2096 |
| 1289 | CCCCACCC C CACCCACU | 763 | ACTCCCTC GCCTAGCTACAACCA CCGTCCCC | 2097 |
| 1292 | CACCCCCA C CCACUCCC | 764 | CCCACTCC CCCTACCTACAACCA TCCCCCTC | 2098 |
| 1298 | CACCCACU C CCCUCUCC | 765 | CCACACCC CCCTACCTACAACCA AGTCCCTC | 2099 |
| 1301 | CCACUCCC C UCUCCCCC | 766 | CGCCCACA CCCTAGCTACAACCA CCCACTCC | 2100 |
| 1307 | CCCUCUCC C CCACCCAC | 767 | CTCCCTCG GGCTACCTACAACCA CCACACGC | 2101 |
| 1312 | UCCCCCAC C CACCCACC | 768 | CCTCCCTC CCCTACCTACAACCA CTCCCCCA | 2102 |
| 1316 | CCACCCAC C CACCCCCC | 769 | CCCCCCTC CCCTAGCTACAACCA CTCCCECC | 2103 |
| 1319 | CCCACCCA C CCCCCCUC | 770 | CACCCCCC CGCTACCTACAACCA TCCCTCCC | 2104 |
| 1325 | CACCCCCC C UCCCCCCA | 771 | TCCCCCCA GGCTACCTACAACCA CCCCCCTC | 2105 |
| 1328 | CCCCCCUC C CCCCACUC | 772 | CACTCCCC CCCTACCTACAACCA CACCCCCC | 2106 |
| 1337 | CCCCACUC C UCACUCAC | 773 | CTCACTCA CCCTACCTACAACCA CACTCCCC | 2107 |
| 1349 | CUCAGAAC C CUCACCUG | 774 | CACCTGAG CGCTAGCTACAACCA CTTCTCAG | 2108 |
| 1355 | AGOCUCAC C UCCCCUAC | 775 | CTAGGCCA GGCTAGCTACAACGA CTGACCCT | 2109 |
| 1357 | CCUCACGU C CCCUACCC | 776 | CGGTACCC GGCTAGCTACAACGA ACCTCACC | 2110 |
| 1367 | CCUACCCA C CCUCACCU | 777 | ACCTCACC CCCTACCTACAACCA TCCCTACC | 2111 |
| 1376 | CCUCACCU C CACCCUCA | 778 | TCACCCTC CCCTACCTACAACCA ACCTCACC | 2112 |
| 1379 | CACCUCCA C CCUCACCC | 779 | CGCTCAGG CCCTAGCTACAACCA TGCACCTG | 2113 |
| 1394 | CCCCCUCC C CCUCCCCC | 780 | CCCCCACC CCCTACCTACAACCA CCACCCCC | 2114 |
| 1399 | UCCGCCUC G CCCUCCUC | 781 | CACCACCG GCCTAGCTACAACCA CACGCCCA | 2115 |
| 1401 | CCCCUCCC C CUCCUCCU | 782 | ACCACCAC CCCTAGCTACAACCA CCCACCCC | 2116 |
| 1405 | UCCCCCUC C UCCUCUCC | 783 | CCACACCA CCCTACCTACAACCA CACCCCCA | 2117 |
| 1407 | GCGCUGGU G CUGUGGAC | 784 | GTCCACAG GGCTAGCTACAACGA ACCAGCGC | 2118 |
| 1410 | CUGGUGCU G UGGACAGU | 785 | ACTGTCCA GGCTAGCTACAACGA AGCACCAG | 2119 |
| 1417 | UGUGGACA U UGCUUGGG | 786 | CCCAAGCA GGCTAGCTACAACGA TGTCCACA | 2120 |
| 1419 | UGGACAGU G CUUGGGCC | 787 | GGCCCAAG GGCTAGCTACAACGA ACTGTCCA | 2121 |
| 1425 | GUGCUUGG C CCCUGCUG | 788 | CAGCAGUG GGCTAGCTACAACGA CCAAGCAC | 2122 |
| 1430 | UGGGCCCU C CUGACCCC | 789 | GGGGTCAG GGCTAGCTACAACGA AGGGCCCA | 2123 |
| 13 | CCCCUACG A UGAAGAGG | 790 | CCTCTTCA GGCTAGCTACAACGA CGTAGGGG | 2124 |
| 116 | AUGCUACA A UGAGCCCA | 791 | TGGGCTCA GGCTAGCTACAACGA TGTAGCAT | 2125 |
| 130 | CCAAGGUG A CGACAAGC | 792 | GCTTGTCG GGCTAGCTACAACGA CACCTTGG | 2126 |
| 133 | AGGUGACG A CAAGCUGC | 793 | GCAGCTTG GGCTAGCTACAACGA CGTCACCT | 2127 |
| 212 | GCACGGCA A CCGCAUCU | 794 | AGATGCGG GGCTAGCTACAACGA TGCCGTGC | 2128 |
| 575 | GCACGGCA A CCGCAUCU | 794 | AGATGCGG GGCTAGCTACAACGA TGCCGTGC | 2129 |
| 257 | CUGCCGCA A CCUCACCA | 795 | TGGTGAGG GGCTAGCTACAACGA TGCGGCAG | 2130 |
| 284 | GCACUCGA A UGUGCUGG | 796 | CCAGCACA GGCTAGCTACAACGA TCGAGTGC | 2131 |
| 298 | UGGCCCGA A UUGAUGCG | 797 | CGCATCAA GGCTAGCTACAACGA TCGGGCCA | 2132 |
| 302 | CCGAAUUG A UGCGGCUG | 798 | CAGCCGCA GGCTAGCTACAACGA CAATTCGG | 2133 |
| 344 | GCAGCUGG A COUCAGOG | 799 | CGCTGAGG GGCTAGCTACAACGA CCAGCTGC | 2134 |
| 353 | CCUCAGCG A UAAUGCAC | 800 | GTGCATTA GGCTAGCTACAACGA CGCTGAGG | 2135 |
| 356 | CAGCGAUA A UCCACAUC | 801 | GCTGTGCA GGCTAGCTACAACGA TATCGCTG | 2136 |
| 377 | GUCUGUGG A CCCUGCCA | 802 | TGGCAGGG GGCTAGCTACAACGA CCACAGAC | 2137 |
| 425 | GCACCUGG A CCGCUGCG | 803 | CGCAGCGG GGCTAGCTACAACGA CCAGGTGC | 2138 |
| 500 | CCUGCAGG A CAACGCGC | 804 | GCGCGTTG GGCTAGCTACAACGA CCTGCAGG | 2139 |
| 503 | GCAGGACA A CGCGCUGC | 805 | GCAGCGCG GGCTAGCTACAACGA TGTCCTGC | 2140 |
| 524 | ACUGCCUG A UGACACCU | 806 | AGGTGTCA GGCTAGCTACAACGA CAGGCAGT | 2141 |
| 527 | GCCUGAUG A CACCUUCC | 807 | GGAAGGTG GGCTAGCTACAACGA CATCAGGC | 2142 |
| 539 | CUUCCGCG A CCUGGGCA | 808 | TGCCCAGG GGCTAGCTACAACGA CGCGGAAG | 2143 |
| 548 | CCUGGGCA A COUCACAC | 809 | GTGTGAGG GGCTAGCTACAACGA TGCCCAGG | 2144 |
| 626 | CAGCCUCG A CCGUCUCC | 810 | GGAGACGG GGCTAGCTACAACGA CGAGGCTG | 2145 |
| 647 | GCACCAGA A CCGCGUGG | 811 | CCACGCGG GGCTAGCTACAACGA TCTGGTGC | 2146 |
| 683 | CUUCCGUG A CCUUGGCC | 812 | GGCCAAGG GGCTAGCTACAACGA CACGGAAG | 2147 |
| 700 | UCCUCAUG A CACUCUAU | 813 | ATAGAGTG GGCTAGCTACAACGA CATUAGUC | 2148 |
| 719 | OCUCUCCA A CAAUCUAU | 814 | ATAGATTG GGCTAGCTACAACGA TGGCAAAC | 2149 |
| 722 | UGCCAACA A UCUAUCAG | 815 | CTGATAGA GGCTAGCTACAACGA TUTTUCCA | 2150 |
| 785 | GAGUCUCA A CGACAACC | 816 | GGTTGTCG GGCTAGCTACAACGA TGAGCCTC | 2151 |
| 788 | GCUCAACG A CAACCCCU | 817 | AGGGGTTG GGCTAGCTACAACGA CGTTGAGC | 2152 |
| 791 | CAACGACA A CCCCUGGG | 818 | CCCAGGGG GGCTAGCTACAACGA TGTCGTTG | 2153 |
| 806 | GOUGUGUG A CUGCCGGG | 819 | CCCGGCAG GGCTAGCTACAACGA CACACACC | 2154 |
| 885 | CUCCCGCA A CUCCUGUC | 820 | GCCAGGCG GGCTAGCTACAACGA TUCUGGAG | 2155 |
| 902 | UGGCCGUG A CCUCAAAC | 821 | GTTTGAGG GGCTAGCTACAACGA CACGGCCA | 2156 |
| 909 | GACCUCAA A CGCCUAGC | 822 | GCTAGGCG GGCTAGCTACAACGA TTGAGGTC | 2157 |
| 923 | AGCUGCCA A UGACCUGC | 823 | GCAGGTCA GGCTAGCTACAACGA TGGCAGCT | 2158 |
| 926 | UGCCAAUG A CCUGCAGG | 824 | CCTGCAGG GGCTAGCTACAACGA CATTUCCA | 2159 |
| 973 | CCAUCUGG A CCGGCAGG | 825 | CCTGCCGG GGCTAGCTACAACGA CCAGATGG | 2160 |
| 989 | GGCCACCG A UGAGGAUC | 826 | GCTCCTCA GGCTAGCTACAACGA CGGTGGCC | 2161 |
| 1028 | CCAGCCAG A UGCCGCUG | 827 | CAUCCUCA GGCTAGCTAUAACGA CTGGCTGG | 2162 |
| 1037 | UGCCGCUG A CAAGGCCU | 828 | AGGCCTTG GGCTAGCTACAACGA CACCUCCA | 2163 |
| 1065 | CCUGGAAG A CCAGCUUC | 829 | GAAGCTGG GGCTAGCTACAACGA CTTCCAGG | 2164 |
| 1082 | GCCAGUCA A UGCGCUGA | 830 | TCAGCGCA GGCTAGCTACAACGA TGCCTGCC | 2165 |
| 1095 | CUGAAGGG A CGCGUGCC | 831 | GGCACGCG GGCTAGCTACAACGA CCCTTCAG | 2166 |
| 1112 | GCCCGGUG A CAGCCCGC | 832 | GCGGGCTG GGCTAGCTACAACGA CACCUGUC | 2167 |
| 1127 | UCCUGUCA A CUCCUCUG | 833 | CAGAGCCG GGCTAGCTACAACGA TGCCCGGC | 2168 |
| 1151 | GCACAUCA A UGACUCAC | 834 | GTGAGTCA GGCTAGCTACAACGA TGATGTGC | 2169 |
| 1154 | CAUCAAUG A CUCACCCU | 835 | AGGGTGAG GGCTAGCTACAACGA CATTGATG | 2170 |
| 1168 | CCUUUGGG A CUCUGCCU | 836 | AGGCAGAG GGCTAGCTACAACGA CCCAAAGG | 2171 |
| 1280 | ACGCAAGA A CCGCACCC | 837 | GGGTGCGG GGCTAGCTACAACGA TCTTGCGT | 2172 |
| 1333 | GUGOCCOG A CUGGUGAC | 838 | GTCACCAG GGCTAGCTACAACGA CCCGCCAC | 2173 |
| 1340 | GACUGGUG A CUCAGAAG | 839 | CTTCTGAG GGCTAGCTACAACGA CACCAGTC | 2174 |
| 1414 | UGCUGUGG A CAGUGCUU | 840 | AAGCACTG GGCTAGCTACAACGA CCACAGCA | 2175 |

Input Sequence = AF283463. Cut Site = R/Y
Arm Length = 8. Core Sequence = GGCTAGCTACAACGA
AF283463 (Homo sapiens Nogo receptor mRNA, complete cds.; 1441 bp)

TABLE VII
Human NOGO Receptor Amberzyme Ribozyme and Substrate Sequence

| | | Seq | | Rz Seq |
|---|---|---|---|---|
| Pos | Substrate | ID | Ribozyme | ID |
| 22 | UGAAGAGG G CGUCCGCU | 547 | AGCGGACG GGAGGAAACUCC CU UCAAGGACAUCGUCCGGG CCUCUUCA | 2176 |
| 24 | AAGAGGGC G UCCGCUGG | 548 | CCAGCGGA GGAGGAAACUCC CU UCAAGGACAUCGUCCGGG GCCCUCUU | 2177 |
| 28 | GGGCGUCC G CUGGAGGG | 549 | CCCUCCAG GGAGGAAACUCC CU UCAAGGACAUCGUCCGGG GGACGCCC | 2178 |
| 38 | UGGAGGGA G CCGGCUGC | 550 | GCAGCCGG GGAGGAAACUCC CU UCAAGGACAUCGUCCGGG UCCCUCCA | 2179 |
| 42 | GGGAGCCG G CUGCUGGC | 551 | GCCAGCAG GGAGGAAACUCC CU UCAAGGACAUCGUCCGGG CGGCUCCC | 2180 |
| 45 | AGCCGGCU G CUGGCAUG | 552 | CAUGCCAG GGAGGAAACUCC CU UCAAGGACAUCGUCCGGG AGCCGGCU | 2181 |
| 49 | GGCUGCUG G CAUGGGUG | 553 | CACCCAUG GGAGGAAACUCC CU UCAAGGACAUCGUCCGGG CAGCAGCC | 2182 |
| 55 | UGGCAUGG G UGCUGUGG | 554 | CCACAGCA GGAGGAAACUCC CU UCAAGGACAUCGUCCGGG CCAUGCCA | 2183 |
| 57 | GCAUGGGU G CUGUGGCU | 555 | AGCCACAG GGAGGAAACUCC CU UCAAGGACAUCGUCCGGG ACCCAUGC | 2184 |
| 60 | UGGGUGCU G UGGCUGCA | 556 | UGCAGCCA GGAGGAAACUCC CU UCAAGGACAUCGUCCGGG AGCACCCA | 2185 |
| 63 | GUGCUGUG G CUGCAGGC | 557 | GCCUGCAG GGAGGAAACUCC CU UCAAGGACAUCGUCCGGG CACAGCAC | 2186 |
| 66 | CUGUGGCU G CAGGCCUG | 558 | CAGGCCUG GGAGGAAACUCC CU UCAAGGACAUCGUCCGGG AGCCACAG | 2187 |
| 70 | GGCUGCAG G CCUGGCAG | 559 | CUGCCAGG GGAGGAAACUCC CU UCAAGGACAUCGUCCGGG CUGCAGCC | 2188 |
| 75 | CAGGCCUG G CAGGUGGC | 560 | GCCACCUG GGAGGAAACUCC CU UCAAGGACAUCGUCCGGG CAGGCCUG | 2189 |
| 79 | CCUGGCAG G UGGCAGCC | 561 | GGCUGCCA GGAGGAAACUCC CU UCAAGGACAUCGUCCGGG CUGCCAGG | 2190 |
| 82 | GGCAGGUG G CAGCCCCA | 562 | UGGGGCUG GGAGGAAACUCC CU UCAAGGACAUCGUCCGGG CACCUGCC | 2191 |
| 85 | AGGUGGCA G CCCCAUGC | 563 | GCAUGGGG GGAGGAAACUCC CU UCAAGGACAUCGUCCGGG UGCCACCU | 2192 |
| 92 | AGCCCCAU G CCCAGGUG | 564 | CACCUGGG GGAGGAAACUCC CU UCAAGGACAUCGUCCGGG AUGGGGCU | 2193 |
| 98 | AUGCCCAG G UGCCUGCG | 565 | CGCAGGCA GGAGGAAACUCC CU UCAAGGACAUCGUCCGGG CUGGGCAU | 2194 |
| 100 | GCCCAGGU G CCUGCGUA | 566 | UACGCAGG GGAGGAAACUCC CU UCAAGGACAUCGUCCGGG ACCUGGGC | 2195 |
| 104 | AGGUGCCU G CGUAUGCU | 567 | AGCAUACG GGAGGAAACUCC CU UCAAGGACAUCGUCCGGG AGGCACCU | 2196 |
| 106 | GUGCCUGC G UAUGCUAC | 568 | GUAGCAUA GGAGGAAACUCC CU UCAAGGACAUCGUCCGGG GCAGGCAC | 2197 |
| 110 | CUGCGUAU G CUACAAUG | 569 | CAUUGUAG GGAGGAAACUCC CU UCAAGGACAUCGUCCGGG AUACGCAG | 2198 |
| 120 | UACAAUGA G CCCAAGGU | 570 | ACCUUGGG GGAGGAAACUCC CU UCAAGGACAUCGUCCGGG UCAUUGUA | 2199 |
| 127 | AGCCCAAG G UGACGACA | 571 | UGUCGUCA GGAGGAAACUCC CU UCAAGGACAUCGUCCGGG CUUGGGCU | 2200 |
| 137 | GACGACAA G CUGCCCCC | 572 | GGGGGCAG GGAGGAAACUCC CU UCAAGGACAUCGUCCGGG UUGUCGUC | 2201 |
| 140 | GACAAGCU G CCCCCAGC | 573 | GCUGGGGG GGAGGAAACUCC CU UCAAGGACAUCGUCCGGG AGCUUGUC | 2202 |
| 147 | UGCCCCCA G CAGGGCCU | 574 | AGGCCCUG GGAGGAAACUCC CU UCAAGGACAUCGUCCGGG UGGGGGCA | 2203 |
| 152 | CCAGCAGG G CCUGCAGG | 575 | CCUGCAGG GGAGGAAACUCC CU UCAAGGACAUCGUCCGGG CCUGCUGG | 2204 |
| 156 | CAGGGCCU G CAGGCUGU | 576 | ACAGCCUG GGAGGAAACUCC CU UCAAGGACAUCGUCCGGG AGGCCCUG | 2205 |
| 160 | GCCUGCAG G CUGUGCCC | 577 | GGGCACAG GGAGGAAACUCC CU UCAAGGACAUCGUCCGGG CUGCAGGC | 2206 |
| 163 | UGCAGGCU G UGCCCGUG | 578 | CACGGGCA GGAGGAAACUCC CU UCAAGGACAUCGUCCGGG AGCCUGCA | 2207 |
| 165 | CAGGCUGU G CCCGUGGG | 579 | CCCACGGG GGAGGAAACUCC CU UCAAGGACAUCGUCCGGG ACAGCCUG | 2208 |
| 169 | CUGUGCCC G UGGGCAUC | 580 | GAUGCCCA GGAGGAAACUCC CU UCAAGGACAUCGUCCGGG GGGCACAG | 2209 |
| 173 | GCCCGUGG G CAUCCCUG | 581 | CAGGGAUG GGAGGAAACUCC CU UCAAGGACAUCGUCCGGG CCACGGGC | 2210 |
| 181 | GCAUCCCU G CUGCCAGC | 582 | GCUGGCAG GGAGGAAACUCC CU UCAAGGACAUCGUCCGGG AGGGAUGC | 2211 |
| 184 | UCCCUGCU G CCAGCCAG | 583 | CUGGCUGG GGAGGAAACUCC CU UCAAGGACAUCGUCCGGG AGCAGGGA | 2212 |
| 188 | UGCUGCCA G CCAGCGCA | 584 | UGCGCUGG GGAGGAAACUCC CU UCAAGGACAUCGUCCGGG UGGCAGCA | 2213 |
| 192 | GCCAGCCA G CGCAUCUU | 585 | AAGAUGCG GGAGGAAACUCC CU UCAAGGACAUCGUCCGGG UGGCUGGC | 2214 |
| 194 | CAGCCAGC G CAUCUUCC | 586 | GGAAGAUG GGAGGAAACUCC CU UCAAGGACAUCGUCCGGG GCUGGCUG | 2215 |
| 204 | AUCUUCCU G CACGGCAA | 587 | UUGCCGUG GGAGGAAACUCC CU UCAAGGACAUCGUCCGGG AGGAAGAU | 2216 |
| 209 | CCUGCACG G CAACCGCA | 588 | UGCGGUUG GGAGGAAACUCC CU UCAAGGACAUCGUCCGGG CGUGCAGG | 2217 |
| 572 | CCUGCACG G CAACCGCA | 588 | UGCGGUUG GGAGGAAACUCC CU UCAAGGACAUCGUCCGGG CGUGCAGG | 2218 |
| 215 | CGGCAACC G CAUCUCGC | 589 | GCGAGAUG GGAGGAAACUCC CU UCAAGGACAUCGUCCGGG GGUUGCCG | 2219 |
| 222 | CGCAUCUC G CAUGUGCC | 590 | GGCACAUG GGAGGAAACUCC CU UCAAGGACAUCGUCCGGG GAGAUGCG | 2220 |
| 226 | UCUCGCAU G UGCCAGCU | 591 | AGCUGGCA GGAGGAAACUCC CU UCAAGGACAUCGUCCGGG AUGCGAGA | 2221 |
| 228 | UCGCAUGU G CCAGCUGC | 592 | GCAGCUGG GGAGGAAACUCC CU UCAAGGACAUCGUCCGGG ACAUGCGA | 2222 |
| 232 | AUGUGCCA G CUGCCAGC | 593 | GCUGGCAG GGAGGAAACUCC CU UCAAGGACAUCGUCCGGG UGGCACAU | 2223 |
| 235 | UGCCAGCU G CCAGCUUC | 594 | GAAGCUGG GGAGGAAACUCC CU UCAAGGACAUCGUCCGGG AGCUGGCA | 2224 |
| 239 | AGCUGCCA G CUUCCGUG | 595 | CACGGAAG GGAGGAAACUCC CU UCAAGGACAUCGUCCGGG UGGCAGCU | 2225 |
| 245 | CAGCUUCC G UGCCUGCC | 596 | GGCAGGCA GGAGGAAACUCC CU UCAAGGACAUCGUCCGGG GGAAGCUG | 2226 |
| 247 | GCUUCCGU G CCUGCCGC | 597 | GCGGCAGG GGAGGAAACUCC CU UCAAGGACAUCGUCCGGG ACGGAAGC | 2227 |
| 251 | CCGUGCCU G CCGCAACC | 598 | GGUUGCGG GGAGGAAACUCC CU UCAAGGACAUCGUCCGGG AGGCACGG | 2228 |
| 254 | UGCCUGCC G CAACCUCA | 599 | UGAGGUUG GGAGGAAACUCC CU UCAAGGACAUCGUCCGGG GGCAGGCA | 2229 |
| 270 | ACCAUCCU G UGGCUGCA | 600 | UGCAGCCA GGAGGAAACUCC CU UCAAGGACAUCGUCCGGG AGGAUGGU | 2230 |
| 273 | AUCCUGUG G CUGCACUC | 601 | GAGUGCAG GGAGGAAACUCC CU UCAAGGACAUCGUCCGGG CACAGGAU | 2231 |
| 276 | CUGUGGCU G CACUCGAA | 602 | UUCGAGUG GGAGGAAACUCC CU UCAAGGACAUCGUCCGGG AGCCACAG | 2232 |
| 286 | ACUCGAAU G UGCUGGCC | 603 | GGCCAGCA GGAGGAAACUCC CU UCAAGGACAUCGUCCGGG AUUCGAGU | 2233 |
| 288 | UCGAAUGU G CUGGCCCG | 604 | CGGGCCAG GGAGGAAACUCC CU UCAAGGACAUCGUCCGGG ACAUUCGA | 2234 |
| 292 | AUGUGCUG G CCCGAAUU | 605 | AAUUCGGG GGAGGAAACUCC CU UCAAGGACAUCGUCCGGG CAGCACAU | 2235 |
| 304 | GAAUUGAU G CGGCUGCC | 606 | GGCAGCCG GGAGGAAACUCC CU UCAAGGACAUCGUCCGGG AUCAAUUC | 2236 |
| 307 | UUGAUGCG G CUGCCUUC | 607 | GAAGGCAG GGAGGAAACUCC CU UCAAGGACAUCGUCCGGG CGCAUCAA | 2237 |
| 310 | AUGCGGCU G CCUUCACU | 608 | AGUGAAGG GGAGGAAACUCC CU UCAAGGACAUCGUCCGGG AGCCGCAU | 2238 |
| 320 | CUUCACUG G CCUGGCCC | 609 | GGGCCAGG GGAGGAAACUCC CU UCAAGGACAUCGUCCGGG CAGUGAAG | 2239 |
| 325 | CUGGCCUG G CCCUCCUG | 610 | CAGGAGGG GGAGGAAACUCC CU UCAAGGACAUCGUCCGGG CAGGCCAG | 2240 |
| 336 | CUCCUGGA G CAGCUGGA | 611 | UCCAGCUG GGAGGAAACUCC CU UCAAGGACAUCGUCCGGG UCCAGGAG | 2241 |
| 339 | CUGGAGCA G CUGGACCU | 612 | AGGUCCAG GGAGGAAACUCC CU UCAAGGACAUCGUCCGGG UGCUCCAG | 2242 |
| 350 | GGACCUCA G CGAUAAUG | 613 | CAUUAUCG GGAGGAAACUCC CU UCAAGGACAUCGUCCGGG UGAGGUCC | 2243 |
| 358 | GCGAUAAU G CACAGCUC | 614 | GAGCUGUG GGAGGAAACUCC CU UCAAGGACAUCGUCCGGG AUUAUCGC | 2244 |
| 363 | AAUGCACA G CUCCGGUC | 615 | GACCGGAG GGAGGAAACUCC CU UCAAGGACAUCGUCCGGG UGUGCAUU | 2245 |
| 369 | CAGCUCCG G UCUGUGGA | 616 | UCCACAGA GGAGGAAACUCC CU UCAAGGACAUCGUCCGGG CGGAGCUG | 2246 |
| 373 | UCCGGUCU G UGGACCCU | 617 | AGGGUCCA GGAGGAAACUCC CU UCAAGGACAUCGUCCGGG AGACCGGA | 2247 |
| 382 | UGGACCCU G CCACAUUC | 618 | GAAUGUGG GGAGGAAACUCC CU UCAAGGACAUCGUCCGGG AGGGUCCA | 2248 |
| 395 | AUUCCACG G CCUGGGCC | 619 | GGCCCAGG GGAGGAAACUCC CU UCAAGGACAUCGUCCGGG CGUGGAAU | 2249 |
| 401 | CGGCCUGG G CCGCCUAC | 620 | GUAGGCGG GGAGGAAACUCC CU UCAAGGACAUCGUCCGGG CCAGGCCG | 2250 |
| 404 | CCUGGGCC G CCUACACA | 621 | UGUGUAGG GGAGGAAACUCC CU UCAAGGACAUCGUCCGGG GGCCCAGG | 2251 |
| 414 | CUACACAC G CUGCACCU | 622 | AGGUGCAG GGAGGAAACUCC CU UCAAGGACAUCGUCCGGG GUGUGUAG | 2252 |
| 417 | CACACGCU G CACCUGGA | 623 | UCCAGGUG GGAGGAAACUCC CU UCAAGGACAUCGUCCGGG AGCGUGUG | 2253 |
| 428 | CCUGGACC G CUGCGGCC | 624 | GGCCGCAG GGAGGAAACUCC CU UCAAGGACAUCGUCCGGG GGUCCAGG | 2254 |
| 431 | GGACCGCU G CGGCCUGC | 625 | GCAGGCCG GGAGGAAACUCC CU UCAAGGACAUCGUCCGGG AGCGGUCC | 2255 |
| 434 | CCGCUGCG G CCUGCAGG | 626 | CCUGCAGG GGAGGAAACUCC CU UCAAGGACAUCGUCCGGG CGCAGCGG | 2256 |
| 438 | UGCGGCCU G CAGGAGCU | 627 | AGCUCCUG GGAGGAAACUCC CU UCAAGGACAUCGUCCGGG AGGCCGCA | 2257 |
| 444 | CUGCAGGA G CUGGGCCC | 628 | GGGCCCAG GGAGGAAACUCC CU UCAAGGACAUCGUCCGGG UCCUGCAG | 2258 |
| 449 | GGAGCUGG G CCCGGGGC | 629 | GCCCCGGG GGAGGAAACUCC CU UCAAGGACAUCGUCCGGG CCAGCUCC | 2259 |
| 456 | GGCCCGGG G CUGUUCCG | 630 | CGGAACAG GGAGGAAACUCC CU UCAAGGACAUCGUCCGGG CCCGGGCC | 2260 |
| 459 | CCGGGGCU G UUCCGCGG | 631 | CCGCGGAA GGAGGAAACUCC CU UCAAGGACAUCGUCCGGG AGCCCCGG | 2261 |
| 464 | GCUGUUCC G CGGCCUGG | 632 | CCAGGCCG GGAGGAAACUCC CU UCAAGGACAUCGUCCGGG GGAACAGC | 2262 |
| 467 | GUUCCGCG G CCUGGCUG | 633 | CAGCCAGG GGAGGAAACUCC CU UCAAGGACAUCGUCCGGG CGCGGAAC | 2263 |
| 472 | GCGGCCUG G CUGCCCUG | 634 | CAGGGCAG GGAGGAAACUCC CU UCAAGGACAUCGUCCGGG CAGGCCGC | 2264 |
| 475 | GCCUGGCU G CCCUGCAG | 635 | CUGCAGGG GGAGGAAACUCC CU UCAAGGACAUCGUCCGGG AGCCAGGC | 2265 |
| 480 | GCUGCCCU G CAGUACCU | 636 | AGGUACUG GGAGGAAACUCC CU UCAAGGACAUCGUCCGGG AGGGCAGC | 2266 |
| 483 | GCCCUGCA G UACCUCUA | 637 | UAGAGGUA GGAGGAAACUCC CU UCAAGGACAUCGUCCGGG UGCAGGGC | 2267 |
| 495 | CUCUACCU G CAGGACAA | 638 | UUGUCCUG GGAGGAAACUCC CU UCAAGGACAUCGUCCGGG AGGUAGAG | 2268 |
| 505 | AGGACAAC G CGCUGCAG | 639 | CUGCAGCG GGAGGAAACUCC CU UCAAGGACAUCGUCCGGG GUUGUCCU | 2269 |
| 507 | GACAACGC G CUGCAGGC | 640 | GCCUGCAG GGAGGAAACUCC CU UCAAGGACAUCGUCCGGG GCGUUGUC | 2270 |
| 510 | AACGCGCU G CAGGCACU | 641 | AGUGCCUG GGAGGAAACUCC CU UCAAGGACAUCGUCCGGG AGCGCGUU | 2271 |
| 514 | CGCUGCAG G CACUGCCU | 642 | AGGCAGUG GGAGGAAACUCC CU UCAAGGACAUCGUCCGGG CUGCAGCG | 2272 |
| 519 | CAGGCACU G CCUGAUGA | 643 | UCAUCAGG GGAGGAAACUCC CU UCAAGGACAUCGUCCGGG AGUGCCUG | 2273 |
| 536 | CACCUUCC G CGACCUGG | 644 | CCAGGUCG GGAGGAAACUCC CU UCAAGGACAUCGUCCGGG GGAAGGUG | 2274 |
| 545 | CGACCUGG G CAACCUCA | 645 | UGAGGUUG GGAGGAAACUCC CU UCAAGGACAUCGUCCGGG CCAGGUCG | 2275 |
| 567 | CUCUUCCU G CACGGCAA | 646 | UUGCCGUG GGAGGAAACUCC CU UCAAGGACAUCGUCCGGG AGGAAGAG | 2276 |
| 578 | CGGCAACC G CAUCUCCA | 647 | UGGAGAUG GGAGGAAACUCC CU UCAAGGACAUCGUCCGGG GGUUGCCG | 2277 |
| 587 | CAUCUCCA G CGUGCCCG | 648 | CGGGCACG GGAGGAAACUCC CU UCAAGGACAUCGUCCGGG UGGAGAUG | 2278 |
| 589 | UCUCCAGC G UGCCCGAG | 649 | CUCGGGCA GGAGGAAACUCC CU UCAAGGACAUCGUCCGGG GCUGGAGA | 2279 |
| 591 | UCCAGCGU G CCCGAGCG | 650 | CGCUCGGG GGAGGAAACUCC CU UCAAGGACAUCGUCCGGG ACGCUGGA | 2280 |
| 597 | GUGCCCGA G CGCGCCUU | 651 | AAGGCGCG GGAGGAAACUCC CU UCAAGGACAUCGUCCGGG UCGGGCAC | 2281 |
| 599 | GCCCGAGC G CGCCUUCC | 652 | GGAAGGCG GGAGGAAACUCC CU UCAAGGACAUCGUCCGGG GCUCGGGC | 2282 |
| 601 | CCGAGCGC G CCUUCCGU | 653 | ACGGAAGG GGAGGAAACUCC CU UCAAGGACAUCGUCCGGG GCGCUCGG | 2283 |
| 608 | CGCCUUCC G UGGGCUGC | 654 | GCAGCCCA GGAGGAAACUCC CU UCAAGGACAUCGUCCGGG GGAAGGCG | 2284 |
| 612 | UUCCGUGG G CUGCACAG | 655 | CUGUGCAG GGAGGAAACUCC CU UCAAGGACAUCGUCCGGG CCACGGAA | 2285 |
| 615 | CGUGGGCU G CACAGCCU | 656 | AGGCUGUG GGAGGAAACUCC CU UCAAGGACAUCGUCCGGG AGCCCACG | 2286 |
| 620 | GCUGCACA G CCUCGACC | 657 | GGUCGAGG GGAGGAAACUCC CU UCAAGGACAUCGUCCGGG UGUGCAGC | 2287 |
| 629 | CCUCGACC G UCUCCUAC | 658 | GUAGGAGA GGAGGAAACUCC CU UCAAGGACAUCGUCCGGG GGUCGAGG | 2288 |
| 639 | CUCCUACU G CACCAGAA | 659 | UUCUGGUG GGAGGAAACUCC CU UCAAGGACAUCGUCCGGG AGUAGGAG | 2289 |
| 650 | CCAGAACC G CGUGGCCC | 660 | GGGCCACG GGAGGAAACUCC CU UCAAGGACAUCGUCCGGG GGUUCUGG | 2290 |
| 652 | AGAACCGC G UGGCCCAU | 661 | AUGGGCCA GGAGGAAACUCC CU UCAAGGACAUCGUCCGGG GCGGUUCU | 2291 |
| 655 | ACCGCGUG G CCCAUGUG | 662 | CACAUGGG GGAGGAAACUCC CU UCAAGGACAUCGUCCGGG CACGCGGU | 2292 |
| 661 | UGGCCCAU G UGCACCCG | 663 | CGGGUGCA GGAGGAAACUCC CU UCAAGGACAUCGUCCGGG AUGGGCCA | 2293 |
| 663 | GCCCAUGU G CACCCGCA | 664 | UGCGGGUG GGAGGAAACUCC CU UCAAGGACAUCGUCCGGG ACAUGGGC | 2294 |
| 669 | GUGCACCC G CAUGCCUU | 665 | AAGGCAUG GGAGGAAACUCC CU UCAAGGACAUCGUCCGGG GGGUGCAC | 2295 |
| 673 | ACCCGCAU G CCUUCCGU | 666 | ACGGAAGG GGAGGAAACUCC CU UCAAGGACAUCGUCCGGG AUGCGGGU | 2296 |
| 680 | UGCCUUCC G UGACCUUG | 667 | CAAGGUCA GGAGGAAACUCC CU UCAAGGACAUCGUCCGGG GGAAGGCA | 2297 |
| 689 | UGACCUUG G CCGCCUCA | 668 | UGAGGCGG GGAGGAAACUCC CU UCAAGGACAUCGUCCGGG CAAGGUCA | 2298 |
| 692 | CCUUGGCC G CCUCAUGA | 669 | UCAUGAGG GGAGGAAACUCC CU UCAAGGACAUCGUCCGGG GGCCAAGG | 2299 |
| 711 | CUCUAUCU G UUUGCCAA | 670 | UUGGCAAA GGAGGAAACUCC CU UCAAGGACAUCGUCCGGG AGAUAGAG | 2300 |
| 715 | AUCUGUUU G CCAACAAU | 671 | AUUGUUGG GGAGGAAACUCC CU UCAAGGACAUCGUCCGGG AAACAGAU | 2301 |
| 730 | AUCUAUCA G CGCUGCCC | 672 | GGGCAGCG GGAGGAAACUCC CU UCAAGGACAUCGUCCGGG UGAUAGAU | 2302 |
| 732 | CUAUCAGC G CUGCCCAC | 673 | GUGGGCAG GGAGGAAACUCC CU UCAAGGACAUCGUCCGGG GCUGAUAG | 2303 |
| 735 | UCAGCGCU G CCCACUGA | 674 | UCAGUGGG GGAGGAAACUCC CU UCAAGGACAUCGUCCGGG AGCGCUGA | 2304 |
| 745 | CCACUGAG G CCCUGGCC | 675 | GGCCAGGG GGAGGAAACUCC CU UCAAGGACAUCGUCCGGG CUCAGUGG | 2305 |
| 751 | AGGCCCUG G CCCCCCUG | 676 | CAGGGGGG GGAGGAAACUCC CU UCAAGGACAUCGUCCGGG CAGGGCCU | 2306 |
| 759 | GCCCCCCU G CGUGCCCU | 677 | AGGGCACG GGAGGAAACUCC CU UCAAGGACAUCGUCCGGG AGGGGGGC | 2307 |
| 761 | CCCCCUGC G UGCCCUGC | 678 | GCAGGGCA GGAGGAAACUCC CU UCAAGGACAUCGUCCGGG GCAGGGGG | 2308 |
| 763 | CCCUGCGU G CCCUGCAG | 679 | CUGCAGGG GGAGGAAACUCC CU UCAAGGACAUCGUCCGGG ACGCAGGG | 2309 |
| 768 | CGUGCCCU G CAGUACCU | 680 | AGGUACUG GGAGGAAACUCC CU UCAAGGACAUCGUCCGGG AGGGCACG | 2310 |
| 771 | GCCCUGCA G UACCUGAG | 681 | CUCAGGUA GGAGGAAACUCC CU UCAAGGACAUCGUCCGGG UGCAGGGC | 2311 |
| 780 | UACCUGAG G CUCAACGA | 682 | UCGUUGAG GGAGGAAACUCC CU UCAAGGACAUCGUCCGGG CUCAGGUA | 2312 |
| 799 | ACCCCUGG G UGUGUGAC | 683 | GUCACACA GGAGGAAACUCC CU UCAAGGACAUCGUCCGGG CCAGGGGU | 2313 |
| 801 | CCCUGGGU G UGUGACUG | 684 | CAGUCACA GGAGGAAACUCC CU UCAAGGACAUCGUCCGGG ACCCAGGG | 2314 |
| 803 | CUGGGUGU G UGACUGCC | 685 | GGCAGUCA GGAGGAAACUCC CU UCAAGGACAUCGUCCGGG ACACCCAG | 2315 |
| 809 | GUGUGACU G CCGGGCAC | 686 | GUGCCCGG GGAGGAAACUCC CU UCAAGGACAUCGUCCGGG AGUCACAC | 2316 |
| 814 | ACUGCCGG G CACGCCCA | 687 | UGGGCGUG GGAGGAAACUCC CU UCAAGGACAUCGUCCGGG CCGGCAGU | 2317 |
| 818 | CCGGGCAC G CCCACUCU | 688 | AGAGUGGG GGAGGAAACUCC CU UCAAGGACAUCGUCCGGG GUGCCCGG | 2318 |
| 829 | CACUCUGG G CCUGGCUG | 689 | CAGCCAGG GGAGGAAACUCC CU UCAAGGACAUCGUCCGGG CCAGAGUG | 2319 |
| 834 | UGGGCCUG G CUGCAGAA | 690 | UUCUGCAG GGAGGAAACUCC CU UCAAGGACAUCGUCCGGG CAGGCCCA | 2320 |
| 837 | GCCUGGCU G CAGAAGUU | 691 | AACUUCUG GGAGGAAACUCC CU UCAAGGACAUCGUCCGGG AGCCAGGC | 2321 |
| 843 | CUGCAGAA G UUCCGCGG | 692 | CCGCGGAA GGAGGAAACUCC CU UCAAGGACAUCGUCCGGG UUCUGCAG | 2322 |
| 848 | GAAGUUCC G CGGCUCCU | 693 | AGGAGCCG GGAGGAAACUCC CU UCAAGGACAUCGUCCGGG GGAACUUC | 2323 |
| 851 | GUUCCGCG G CUCCUCCU | 694 | AGGAGGAG GGAGGAAACUCC CU UCAAGGACAUCGUCCGGG CGCGGAAC | 2324 |
| 865 | CCUCCGAG G UGCCCUGC | 695 | GCAGGGCA GGAGGAAACUCC CU UCAAGGACAUCGUCCGGG CUCGGAGG | 2325 |
| 867 | UCCGAGGU G CCCUGCAG | 696 | CUGCAGGG GGAGGAAACUCC CU UCAAGGACAUCGUCCGGG ACCUCGGA | 2326 |
| 872 | GGUGCCCU G CAGCCUCC | 697 | GGAGGCUG GGAGGAAACUCC CU UCAAGGACAUCGUCCGGG AGGGCACC | 2327 |
| 875 | GCCCUGCA G CCUCCCGC | 698 | GCGGGAGG GGAGGAAACUCC CU UCAAGGACAUCGUCCGGG UGCAGGGC | 2328 |
| 882 | AGCCUCCC G CAACGCCU | 699 | AGGCGUUG GGAGGAAACUCC CU UCAAGGACAUCGUCCGGG GGGAGGCU | 2329 |
| 887 | CCCGCAAC G CCUGGCUG | 700 | CAGCCAGG GGAGGAAACUCC CU UCAAGGACAUCGUCCGGG GUUGCGGG | 2330 |
| 892 | AACGCCUG G CUGGCCGU | 701 | ACGGCCAG GGAGGAAACUCC CU UCAAGGACAUCGUCCGGG CAGGCGUU | 2331 |
| 896 | CCUGGCUG G CCGUGACC | 702 | GGUCACGG GGAGGAAACUCC CU UCAAGGACAUCGUCCGGG CAGCCAGG | 2332 |
| 899 | GGCUGGCC G UGACCUCA | 703 | UGAGGUCA GGAGGAAACUCC CU UCAAGGACAUCGUCCGGG GGCCAGCC | 2333 |
| 911 | CCUCAAAC G CCUAGCUG | 704 | CAGCUAGG GGAGGAAACUCC CU UCAAGGACAUCGUCCGGG GUUUGAGG | 2334 |
| 916 | AACGCCUA G CUGCCAAU | 705 | AUUGGCAG GGAGGAAACUCC CU UCAAGGACAUCGUCCGGG UAGGCGUU | 2335 |
| 919 | GCCUAGCU G CCAAUGAC | 706 | GUCAUUGG GGAGGAAACUCC CU UCAAGGACAUCGUCCGGG AGCUAGGC | 2336 |
| 930 | AAUGACCU G CAGGGCUG | 707 | CAGCCCUG GGAGGAAACUCC CU UCAAGGACAUCGUCCGGG AGGUCAUU | 2337 |
| 935 | CCUGCAGG G CUGCGCUG | 708 | CAGCGCAG GGAGGAAACUCC CU UCAAGGACAUCGUCCGGG CCUGCAGG | 2338 |
| 938 | GCAGGGCU G CGCUGUGG | 709 | CCACAGCG GGAGGAAACUCC CU UCAAGGACAUCGUCCGGG AGCCCUGC | 2339 |
| 940 | AGGGCUGC G CUGUGGCC | 710 | GGCCACAG GGAGGAAACUCC CU UCAAGGACAUCGUCCGGG GCAGCCCU | 2340 |
| 943 | GCUGCGCU G UGGCCACC | 711 | GGUGGCCA GGAGGAAACUCC CU UCAAGGACAUCGUCCGGG AGCGCAGC | 2341 |
| 946 | GCGCUGUG G CCACCGGC | 712 | GCCGGUGG GGAGGAAACUCC CU UCAAGGACAUCGUCCGGG CACAGCGC | 2342 |
| 953 | GGCCACCG G CCCUUACC | 713 | GGUAAGGG GGAGGAAACUCC CU UCAAGGACAUCGUCCGGG CGGUGGCC | 2343 |
| 977 | CUGGACCG G CAGGGCCA | 714 | UGGCCCUG GGAGGAAACUCC CU UCAAGGACAUCGUCCGGG CGGUCCAG | 2344 |
| 982 | CCGGCAGG G CCACCGAU | 715 | AUCGGUGG GGAGGAAACUCC CU UCAAGGACAUCGUCCGGG CCUGCCGG | 2345 |
| 996 | GAUGAGGA G CCGCUGGG | 716 | CCCAGCGG GGAGGAAACUCC CU UCAAGGACAUCGUCCGGG UCCUCAUC | 2346 |
| 1005 | CCGCUGGG G CUUCCCAA | 718 | UUGGGAAG GGAGGAAACUCC CU UCAAGGACAUCGUCCGGG CCCAGCGG | 2348 |
| 1014 | CUUCCCAA G UGCUGCCA | 719 | UGGCAGCA GGAGGAAACUCC CU UCAAGGACAUCGUCCGGG UUGGGAAG | 2349 |
| 1016 | UCCCAAGU G CUGCCAGC | 720 | GCUGGCAG GGAGGAAACUCC CU UCAAGGACAUCGUCCGGG ACUUGGGA | 2350 |
| 1019 | CAAGUGCU G CCAGCCAG | 721 | CUGGCUGG GGAGGAAACUCC CU UCAAGGACAUCGUCCGGG AGCACUUG | 2351 |
| 1023 | UGCUGCCA G CCAGAUGC | 722 | GCAUCUGG GGAGGAAACUCC CU UCAAGGACAUCGUCCGGG UGGCAGCA | 2352 |
| 1030 | AGCCAGAU G CCGCUGAC | 723 | GUCAGCGG GGAGGAAACUCC CU UCAAGGACAUCGUCCGGG AUCUGGCU | 2353 |
| 1033 | CAGAUGCC G CUGACAAG | 724 | CUUGUCAG GGAGGAAACUCC CU UCAAGGACAUCGUCCGGG GGCAUCUG | 2354 |
| 1042 | CUGACAAG G CCUCAGUA | 725 | UACUGAGG GGAGGAAACUCC CU UCAAGGACAUCGUCCGGG CUUGUCAG | 2355 |
| 1048 | AGGCCUCA G UACUGGAG | 726 | CUCCAGUA GGAGGAAACUCC CU UCAAGGACAUCGUCCGGG UGAGGCCU | 2356 |
| 1056 | GUACUGGA G CCUGGAAG | 727 | CUUCCAGG GGAGGAAACUCC CU UCAAGGACAUCGUCCGGG UCCAGUAC | 2357 |
| 1069 | GAAGACCA G CUUCGGCA | 728 | UGCCGAAG GGAGGAAACUCC CU UCAAGGACAUCGUCCGGG UGGUCUUC | 2358 |
| 1075 | CAGCUUCG G CAGGCAAU | 729 | AUUGCCUG GGAGGAAACUCC CU UCAAGGACAUCGUCCGGG CGAAGCUG | 2359 |
| 1079 | UUCGGCAG G CAAUGCGC | 730 | GCGCAUUG GGAGGAAACUCC CU UCAAGGACAUCGUCCGGG CUGCCGAA | 2360 |
| 1084 | CAGGCAAU G CGCUGAAG | 731 | CUUCAGCG GGAGGAAACUCC CU UCAAGGACAUCGUCCGGG AUUGCCUG | 2361 |
| 1086 | GGCAAUGC G CUGAAGGG | 732 | CCCUUCAG GGAGGAAACUCC CU UCAAGGACAUCGUCCGGG GCAUUGCC | 2362 |
| 1097 | GAAGGGAC G CGUGCCGC | 733 | GCGGCACG GGAGGAAACUCC CU UCAAGGACAUCGUCCGGG GUCCCUUC | 2363 |
| 1099 | AGGGACGC G UGCCGCCC | 734 | GGGCGGCA GGAGGAAACUCC CU UCAAGGACAUCGUCCGGG GCGUCCCU | 2364 |
| 1101 | GGACGCGU G CCGCCCGG | 735 | CCGGGCGG GGAGGAAACUCC CU UCAAGGACAUCGUCCGGG ACGCGUCC | 2365 |
| 1104 | CGCGUGCC G CCCGGUGA | 736 | UCACCGGG GGAGGAAACUCC CU UCAAGGACAUCGUCCGGG GGCACGCG | 2366 |
| 1109 | GCCGCCCG G UGACAGCC | 737 | GGCUGUCA GGAGGAAACUCC CU UCAAGGACAUCGUCCGGG CGGGCGGC | 2367 |
| 1115 | CGGUGACA G CCCGCCGG | 738 | CCGGCGGG GGAGGAAACUCC CU UCAAGGACAUCGUCCGGG UGUCACCG | 2368 |
| 1119 | GACAGCCC G CCGGGCAA | 739 | UUGCCCGG GGAGGAAACUCC CU UCAAGGACAUCGUCCGGG GGGCUGUC | 2369 |
| 1124 | CCCGCCGG G CAACGGCU | 740 | AGCCGUUG GGAGGAAACUCC CU UCAAGGACAUCGUCCGGG CCGGCGGG | 2370 |
| 1130 | GGGCAACG G CUCUGGCC | 741 | GGCCAGAG GGAGGAAACUCC CU UCAAGGACAUCGUCCGGG CGUUGCCC | 2371 |
| 1136 | CGGCUCUG G CCCACGGC | 742 | GCCGUGGG GGAGGAAACUCC CU UCAAGGACAUCGUCCGGG CAGAGCCG | 2372 |
| 1143 | GGCCCACG G CACAUCAA | 743 | UUGAUGUG GGAGGAAACUCC CU UCAAGGACAUCGUCCGGG CGUGGGCC | 2373 |
| 1173 | GGGACUCU G CCUGGCUC | 744 | GAGCCAGG GGAGGAAACUCC CU UCAAGGACAUCGUCCGGG AGAGUCCC | 2374 |
| 1178 | UCUGCCUG G CUCUGCUG | 745 | CAGCAGAG GGAGGAAACUCC CU UCAAGGACAUCGUCCGGG CAGGCAGA | 2375 |
| 1183 | CUGGCUCU G CUGAGCCC | 746 | GGGCUCAG GGAGGAAACUCC CU UCAAGGACAUCGUCCGGG AGAGCCAG | 2376 |
| 1188 | UCUGCUGA G CCCCCGCU | 747 | AGCGGGGG GGAGGAAACUCC CU UCAAGGACAUCGUCCGGG UCAGCAGA | 2377 |
| 1194 | GAGCCCCC G CUCACUGC | 748 | GCAGUGAG GGAGGAAACUCC CU UCAAGGACAUCGUCCGGG GGGGGCUC | 2378 |
| 1201 | CGCUCACU G CAGUGCGG | 749 | CCGCACUG GGAGGAAACUCC CU UCAAGGACAUCGUCCGGG AGUGAGCG | 2379 |
| 1204 | UCACUGCA G UGCGGCCC | 750 | GGGCCGCA GGAGGAAACUCC CU UCAAGGACAUCGUCCGGG UGCAGUGA | 2380 |
| 1206 | ACUGCAGU G CGGCCCGA | 751 | UCGGGCCG GGAGGAAACUCC CU UCAAGGACAUCGUCCGGG ACUGCAGU | 2381 |
| 1209 | GCAGUGCG G CCCGAGGG | 752 | CCCUCGGG GGAGGAAACUCC CU UCAAGGACAUCGUCCGGG CGCACUGC | 2382 |
| 1217 | GCCCGAGG G CUCCGAGC | 753 | GCUCGGAG GGAGGAAACUCC CU UCAAGGACAUCGUCCGGG CCUCGGGC | 2383 |
| 1224 | GGCUCCGA G CCACCAGG | 754 | CCUGGUGG GGAGGAAACUCC CU UCAAGGACAUCGUCCGGG UCGGAGCC | 2384 |
| 1233 | CCACCAGG G UUCCCCAC | 755 | GUGGGGAA GGAGGAAACUCC CU UCAAGGACAUCGUCCGGG CCUGGUGG | 2385 |
| 1247 | CACCUCGG G CCCUCGCC | 756 | GGCGAGGG GGAGGAAACUCC CU UCAAGGACAUCGUCCGGG CCGAGGUG | 2386 |
| 1253 | GGGCCCUC G CCGGAGGC | 757 | GCCUCCGG GGAGGAAACUCC CU UCAAGGACAUCGUCCGGG GAGGGCCC | 2387 |
| 1260 | CGCCGGAG G CCAGGCUG | 758 | CAGCCUGG GGAGGAAACUCC CU UCAAGGACAUCGUCCGGG CUCCGGCG | 2388 |
| 1265 | GAGGCCAG G CUGUUCAC | 759 | GUGAACAG GGAGGAAACUCC CU UCAAGGACAUCGUCCGGG CUGGCCUC | 2389 |
| 1268 | GCCAGGCU G UUCACGCA | 760 | UGCGUGAA GGAGGAAACUCC CU UCAAGGACAUCGUCCGGG AGCCUGGC | 2390 |
| 1274 | CUGUUCAC G CAAGAACC | 761 | GGUUCUUG GGAGGAAACUCC CU UCAAGGACAUCGUCCGGG GUGAACAG | 2391 |
| 1283 | CAAGAACC G CACCCGCA | 762 | UGCGGGUG GGAGGAAACUCC CU UCAAGGACAUCGUCCGGG GGUUCUUG | 2392 |
| 1289 | CCGCACCC G CAGCCACU | 763 | AGUGGCUG GGAGGAAACUCC CU UCAAGGACAUCGUCCGGG GGGUGCGG | 2393 |
| 1292 | CACCCGCA G CCACUGCC | 764 | GGCAGUGG GGAGGAAACUCC CU UCAAGGACAUCGUCCGGG UGCGGGUG | 2394 |
| 1298 | CAGCCACU G CCGUCUGG | 765 | CCAGACGG GGAGGAAACUCC CU UCAAGGACAUCGUCCGGG AGUGGCUG | 2395 |
| 1301 | CCACUGCC G UCUGGGCC | 766 | GGCCCAGA GGAGGAAACUCC CU UCAAGGACAUCGUCCGGG GGCAGUGG | 2396 |
| 1307 | CCGUCUGG G CCAGGCAG | 767 | CUGCCUGG GGAGGAAACUCC CU UCAAGGACAUCGUCCGGG CCAGACGG | 2397 |
| 1312 | UGGGCCAG G CAGGCAGC | 768 | GCUGCCUG GGAGGAAACUCC CU UCAAGGACAUCGUCCGGG CUGGCCCA | 2398 |
| 1316 | CCAGGCAG G CAGCGGGG | 769 | CCCCGCUG GGAGGAAACUCC CU UCAAGGACAUCGUCCGGG CUGCCUGG | 2399 |
| 1319 | GGCAGGCA G CGGGGGUG | 770 | CACCCCCG GGAGGAAACUCC CU UCAAGGACAUCGUCCGGG UGCCUGCC | 2400 |
| 1325 | CAGCGGGG G UGGCGGGA | 771 | UCCCGCCA GGAGGAAACUCC CU UCAAGGACAUCGUCCGGG CCCCGCUG | 2401 |
| 1328 | CGGGGGUG G CGGGACUG | 772 | CAGUCCCG GGAGGAAACUCC CU UCAAGGACAUCGUCCGGG CACCCCCG | 2402 |
| 1337 | CGGGACUG G UGACUCAG | 773 | CUGAGUCA GGAGGAAACUCC CU UCAAGGACAUCGUCCGGG CAGUCCCG | 2403 |
| 1349 | CUCAGAAG G CUCAGGUG | 774 | CACCUGAG GGAGGAAACUCC CU UCAAGGACAUCGUCCGGG CUUCUGAG | 2404 |
| 1355 | AGGCUCAG G UGCCCUAC | 775 | GUAGGGCA GGAGGAAACUCC CU UCAAGGACAUCGUCCGGG CUGAGCCU | 2405 |
| 1357 | GCUCAGGU G CCCUACCC | 776 | GGGUAGGG GGAGGAAACUCC CU UCAAGGACAUCGUCCGGG ACCUGAGC | 2406 |
| 1367 | CCUACCCA G CCUCACCU | 777 | AGGUGAGG GGAGGAAACUCC CU UCAAGGACAUCGUCCGGG UGGGUAGG | 2407 |
| 1376 | CCUCACCU G CAGCCUCA | 778 | UGAGGCUG GGAGGAAACUCC CU UCAAGGACAUCGUCCGGG AGGUGAGG | 2408 |
| 1379 | CACCUGCA G CCUCACCC | 779 | GGGUGAGG GGAGGAAACUCC CU UCAAGGACAUCGUCCGGG UGCAGGUG | 2409 |
| 1394 | CCCCCUGG G CCUGGCGC | 780 | GCGCCAGG GGAGGAAACUCC CU UCAAGGACAUCGUCCGGG CCAGGGGG | 2410 |
| 1399 | UGGGCCUG G CGCUGGUG | 781 | CACCAGCG GGAGGAAACUCC CU UCAAGGACAUCGUCCGGG CAGGCCCA | 2411 |
| 1401 | GGCCUGGC G CUGGUGCU | 782 | AGCACCAG GGAGGAAACUCC CU UCAAGGACAUCGUCCGGG GCCAGGCC | 2412 |
| 1405 | UGGCGCUG G UGCUGUGG | 783 | CCACAGCA GGAGGAAACUCC CU UCAAGGACAUCGUCCGGG CAGCGCCA | 2413 |
| 1407 | GCGCUGGU G CUGUGGAC | 784 | GUCCACAG GGAGGAAACUCC CU UCAAGGACAUCGUCCGGG ACCAGCGC | 2414 |
| 1410 | CUGGUGCU G UGGACAGU | 785 | ACUGUCCA GGAGGAAACUCC CU UCAAGGACAUCGUCCGGG AGCACCAG | 2415 |
| 1417 | UGUGGACA G UGCUUGGG | 786 | CCCAAGCA GGAGGAAACUCC CU UCAAGGACAUCGUCCGGG UGUCCACA | 2416 |
| 1419 | UGGACAGU G CUUGGGCC | 787 | GGCCCAAG GGAGGAAACUCC CU UCAAGGACAUCGUCCGGG ACUGUCCA | 2417 |
| 1425 | GUGCUUGG G CCCUGCUG | 788 | CAGCAGGG GGAGGAAACUCC CU UCAAGGACAUCGUCCGGG CCAAGCAC | 2418 |
| 1430 | UGGGCCCU G CUGACCCC | 789 | GGGGUCAG GGAGGAAACUCC CU UCAAGGACAUCGUCCGGG AGGGCCCA | 2419 |
| 12 | ACCCCUAC G AUGAAGAG | 841 | CUCUUCAU GGAGGAAACUCC CU UCAAGGACAUCGUCCGGG GUAGGGGU | 2420 |
| 15 | CCUACGAU G AAGACGGC | 842 | GCCCUCUU GGAGGAAACUCC CU UCAAGGACAUCGUCCGGG AUCGUAGG | 2421 |
| 18 | ACGAUGAA G AGGGCGUC | 843 | GACGCCCU GGAGGAAACUCC CU UCAAGGACAUCGUCCGGG UUCAUCGU | 2422 |
| 20 | GAUGAAGA G GGCGUCCG | 844 | CGGACGCC GGAGGAAACUCC CU UCAAGGACAUCGUCCGGG UCUUCAUC | 2423 |
| 21 | AUGAAGAG G GCGUCCGC | 845 | GCGGACGC GGAGGAAACUCC CU UCAAGGACAUCGUCCGGG CUCUUCAU | 2424 |
| 31 | CGUCCGCU G GAGGGAGC | 846 | GCUCCCUC GGAGGAAACUCC CU UCAAGGACAUCGUCCGGG AGCGGACG | 2425 |
| 32 | GUCCGCUG G AGGGAGCC | 847 | GGCUCCCU GGAGGAAACUCC CU UCAAGGACAUCGUCCGGG CAGCGGAC | 2426 |
| 34 | CCGCUGGA G GGAGCCGG | 848 | CCGGCUCC GGAGGAAACUCC CU UCAAGGACAUCGUCCGGG UCCAGCGG | 2427 |
| 35 | CGCUGGAG G GAGCCGGC | 849 | GCCGGCUC GGAGGAAACUCC CU UCAAGGACAUCGUCCGGG CUCCAGCG | 2428 |
| 36 | GCUGGAGG G AGCCGGCU | 850 | AGCCGGCU GGAGGAAACUCC CU UCAAGGACAUCGUCCGGG CCUCCAGC | 2429 |
| 41 | AGGGAGCC G GCUGCUGG | 851 | CCAGCAGC GGAGGAAACUCC CU UCAAGGACAUCGUCCGGG GGCUCCCU | 2430 |
| 48 | CGGCUGCU G GCAUGGGU | 852 | ACCCAUGC GGAGGAAACUCC CU UCAAGGACAUCGUCCGGG AGCAGCCG | 2431 |
| 53 | GCUGGCAU G GGUGCUGU | 853 | ACAGCACC GGAGGAAACUCC CU UCAAGGACAUCGUCCGGG AUGCCAGC | 2432 |
| 54 | CUGGCAUG G GUGCUGUG | 854 | CACAGCAC GGAGGAAACUCC CU UCAAGGACAUCGUCCGGG CAUGCCAG | 2433 |
| 62 | GGUGCUGU G GCUGCAGG | 855 | CCUGCAGC GGAGGAAACUCC CU UCAAGGACAUCGUCCGGG ACAGCACC | 2434 |
| 69 | UGGCUGCA G GCCUGGCA | 856 | UGCCAGGC GGAGGAAACUCC CU UCAAGGACAUCGUCCGGG UGCAGCCA | 2435 |
| 74 | GCAGGCCU G GCAGGUGG | 857 | CCACCUGC GGAGGAAACUCC CU UCAAGGACAUCGUCCGGG AGGCCUGC | 2436 |
| 78 | GCCUGGCA G GUGGCAGC | 858 | GCUGCCAC GGAGGAAACUCC CU UCAAGGACAUCGUCCGGG UGCCAGGC | 2437 |
| 81 | UGGCAGGU G GCAGCCCC | 859 | GGGGCUGC GGAGGAAACUCC CU UCAAGGACAUCGUCCGGG ACCUGCCA | 2438 |
| 97 | CAUGCCCA G GUGCCUGC | 860 | GCAGGCAC GGAGGAAACUCC CU UCAAGGACAUCGUCCGGG UGGGCAUG | 2439 |
| 118 | GCUACAAU G AGCCCAAG | 861 | CUUGGGCU GGAGGAAACUCC CU UCAAGGACAUCGUCCGGG AUUGUAGC | 2440 |
| 126 | GAGCCCAA G GUGACGAC | 862 | GUCGUCAC GGAGGAAACUCC CU UCAAGGACAUCGUCCGGG UUGGGCUC | 2441 |
| 129 | CCCAAGGU G ACGACAAG | 863 | CUUGUCGU GGAGGAAACUCC CU UCAAGGACAUCGUCCGGG ACCUUGGG | 2442 |
| 132 | AAGGUGAC G ACAAGCUG | 864 | CAGCUUGU GGAGGAAACUCC CU UCAAGGACAUCGUCCGGG GUCACCUU | 2443 |
| 150 | CCCCAGCA G GGCCUGCA | 865 | UGCAGGCC GGAGGAAACUCC CU UCAAGGACAUCGUCCGGG UGCUGGGG | 2444 |
| 151 | CCCAGCAG G GCCUGCAG | 866 | CUGCAGGC GGAGGAAACUCC CU UCAAGGACAUCGUCCGGG CUGCUGGG | 2445 |
| 159 | GGCCUGCA G GCUGUGCC | 867 | GGCACAGC GGAGGAAACUCC CU UCAAGGACAUCGUCCGGG UGCAGGCC | 2446 |
| 171 | GUGCCCGU G GGCAUCCC | 868 | GGGAUGCC GGAGGAAACUCC CU UCAAGGACAUCGUCCGGG ACGGGCAC | 2447 |
| 172 | UGCCCGUG G GCAUCCCU | 869 | AGGGAUGC GGAGGAAACUCC CU UCAAGGACAUCGUCCGGG CACGGGCA | 2448 |
| 208 | UCCUGCAC G GCAACCGC | 870 | GCGGUUGC GGAGGAAACUCC CU UCAAGGACAUCGUCCGGG GUGCAGGA | 2449 |
| 571 | UCCUGCAC G GCAACCGC | 870 | GCGGUUGC GGAGGAAACUCC CU UCAAGGACAUCGUCCGGG GUGCAGGA | 2450 |
| 272 | CAUCCUGU G GCUGCACU | 871 | AGUGCAGC GGAGGAAACUCC CU UCAAGGACAUCGUCCGGG ACAGGAUG | 2451 |
| 282 | CUGCACUC G AAUGUGCU | 872 | AGCACAUU GGAGGAAACUCC CU UCAAGGACAUCGUCCGGG GAGUGCAG | 2452 |
| 291 | AAUGUGCU G GCCCGAAU | 873 | AUUCGGGC GGAGGAAACUCC CU UCAAGGACAUCGUCCGGG AGCACAUU | 2453 |
| 296 | GCUGGCCC G AAUUGAUG | 874 | CAUCAAUU GGAGGAAACUCC CU UCAAGGACAUCGUCCGGG GGGCCAGC | 2454 |
| 301 | CCCGAAUU G AUGCGGCU | 875 | AGCCGCAU GGAGGAAACUCC CU UCAAGGACAUCGUCCGGG AAUUCGGG | 2455 |
| 306 | AUUGAUGC G GCUGCCUU | 876 | AAGGCAGC GGAGGAAACUCC CU UCAAGGACAUCGUCCGGG GCAUCAAU | 2456 |
| 319 | CCUUCACU G GCCUGGCC | 877 | GGCCAGGC GGAGGAAACUCC CU UCAAGGACAUCGUCCGGG AGUGAAGG | 2457 |
| 324 | ACUGGCCU G GCCCUCCU | 878 | AGGAGGGC GGAGGAAACUCC CU UCAAGGACAUCGUCCGGG AGGCCAGU | 2458 |
| 333 | GCCCUCCU G GAGCAGCU | 879 | AGCUGCUC GGAGGAAACUCC CU UCAAGGACAUCGUCCGGG AGGAGGGC | 2459 |
| 334 | CCCUCCUG G AGCAGCUG | 880 | CAGCUGCU GGAGGAAACUCC CU UCAAGGACAUCGUCCGGG CAGGAGGG | 2460 |
| 342 | GAGCAGCU G GACCUCAG | 881 | CUGAGGUC GGAGGAAACUCC CU UCAAGGACAUCGUCCGGG AGCUGCUC | 2461 |
| 343 | AGCAGCUG G ACCUCAGC | 882 | GCUGAGGU GGAGGAAACUCC CU UCAAGGACAUCGUCCGGG CAGCUGCU | 2462 |
| 352 | ACCUCAGC G AUAAUGCA | 883 | UGCAUUAU GGAGGAAACUCC CU UCAAGGACAUCGUCCGGG GCUGAGGU | 2463 |
| 368 | ACAGCUCC G GUCUGUGG | 884 | CCACAGAC GGAGGAAACUCC CU UCAAGGACAUCGUCCGGG GGAGCUGU | 2464 |
| 375 | CGGUCUGU G GACCCUGC | 885 | GCAGGGUC GGAGGAAACUCC CU UCAAGGACAUCGUCCGGG ACAGACCG | 2465 |
| 376 | GGUCUGUG G ACCCUGCC | 886 | GGCAGGGU GGAGGAAACUCC CU UCAAGGACAUCGUCCGGG CACAGACC | 2466 |
| 394 | CAUUCCAC G GCCUGGGC | 887 | GCCCAGGC GGAGGAAACUCC CU UCAAGGACAUCGUCCGGG GUGGAAUG | 2467 |
| 399 | CACGGCCU G GGCCGCCU | 888 | AGGCGGCC GGAGGAAACUCC CU UCAAGGACAUCGUCCGGG AGGCCGUG | 2468 |
| 400 | ACGGCCUG G GCCGCCUA | 889 | AGGCGGCC GGAGGAAACUCC CU UCAAGGACAUCGUCCGGG CAGGCCGU | 2469 |
| 423 | CUGCACCU G GACCGCUG | 890 | CAGCGGUC GGAGGAAACUCC CU UCAAGGACAUCGUCCGGG AGGUGCAG | 2470 |
| 424 | UGCACCUG G ACCGCUGC | 891 | GCAGCGGU GGAGGAAACUCC CU UCAAGGACAUCGUCCGGG CAGGUGCA | 2471 |
| 433 | ACCGCUGC G GCCUGCAG | 892 | CUGCAGGC GGAGGAAACUCC CU UCAAGGACAUCGUCCGGG GCAGCGGU | 2472 |
| 441 | GGCCUGCA G GAGCUGGG | 893 | CCCAGCUC GGAGGAAACUCC CU UCAAGGACAUCGUCCGGG UGCAGGCC | 2473 |
| 442 | GCCUGCAG G AGCUGGGC | 894 | GCCCAGCU GGAGGAAACUCC CU UCAAGGACAUCGUCCGGG CUGCAGGC | 2474 |
| 447 | CAGGAGCU G GGCCCGGG | 895 | CCCGGGCC GGAGGAAACUCC CU UCAAGGACAUCGUCCGGG AGCUCCUG | 2475 |
| 448 | AGGAGCUG G GCCCGGGG | 896 | CCCCGGGC GGAGGAAACUCC CU UCAAGGACAUCGUCCGGG CAGCUCCU | 2476 |
| 453 | CUGGGCCC G GGGCUGUU | 897 | AACAGCCC GGAGGAAACUCC CU UCAAGGACAUCGUCCGGG GGGCCCAG | 2477 |
| 454 | UGGGCCCG G GGCUGUUC | 898 | GAACAGCC GGAGGAAACUCC CU UCAAGGACAUCGUCCGGG CGGGCCCA | 2478 |
| 455 | GGGCCCGG G GCUGUUCC | 899 | GGAACAGC GGAGGAAACUCC CU UCAAGGACAUCGUCCGGG CCGGGCCC | 2479 |
| 466 | UGUUCCGC G GCCUGGCU | 900 | AGCCAGGC GGAGGAAACUCC CU UCAAGGACAUCGUCCGGG GCGGAACA | 2480 |
| 471 | CGCGGCCU G GCUGCCCU | 901 | AGGGCAGC GGAGGAAACUCC CU UCAAGGACAUCGUCCGGG AGGCCGCG | 2481 |
| 498 | UACCUGCA G GACAACGC | 902 | GCGUUGUC GGAGGAAACUCC CU UCAAGGACAUCGUCCGGG UGCAGGUA | 2482 |
| 499 | ACCUGCAG G ACAACGCG | 903 | CGCGUUGU GGAGGAAACUCC CU UCAAGGACAUCGUCCGGG CUGCAGGU | 2483 |
| 513 | GCGCUGCA G GCACUGCC | 904 | GGCAGUGC GGAGGAAACUCC CU UCAAGGACAUCGUCCGGG UGCAGCGC | 2484 |
| 523 | CACUGCCU G AUGACACC | 905 | GGUGUCAU GGAGGAAACUCC CU UCAAGGACAUCGUCCGGG AGGCAGUG | 2485 |
| 526 | UGCCUGAU G ACACCUUC | 906 | GAAGGUGU GGAGGAAACUCC CU UCAAGGACAUCGUCCGGG AUCAGGCA | 2486 |
| 538 | CCUUCCGC G ACCUGGGC | 907 | GCCCAGGU GGAGGAAACUCC CU UCAAGGACAUCGUCCGGG GCGGAAGG | 2487 |
| 543 | CGCGACCU G GGCAACCU | 908 | AGGUUGCC GGAGGAAACUCC CU UCAAGGACAUCGUCCGGG AGGUCGCG | 2488 |
| 544 | GCGACCUG G GCAACCUC | 909 | GAGGUUGC GGAGGAAACUCC CU UCAAGGACAUCGUCCGGG CAGGUCGC | 2489 |
| 595 | GCGUGCCC G AGCGCGCC | 910 | GGCGCGCU GGAGGAAACUCC CU UCAAGGACAUCGUCCGGG GGGCACGC | 2490 |
| 610 | CCUUCCGU G GGCUGCAC | 911 | GUGCAGCC GGAGGAAACUCC CU UCAAGGACAUCGUCCGGG ACGGAAGG | 2491 |
| 611 | CUUCCGUG G GCUGCACA | 912 | UGUGCAGC GGAGGAAACUCC CU UCAAGGACAUCGUCCGGG CACGGAAG | 2492 |
| 625 | ACAGCCUC G ACCGUCUC | 913 | GAGACGGU GGAGGAAACUCC CU UCAAGGACAUCGUCCGGG GAGGCUGU | 2493 |
| 645 | CUGCACCA G AACCGCGU | 914 | ACGCGGUU GGAGGAAACUCC CU UCAAGGACAUCGUCCGGG UGGUGCAG | 2494 |
| 654 | AACCGCGU G GCCCAUGU | 915 | ACAUGGGC GGAGGAAACUCC CU UCAAGGACAUCGUCCGGG ACGCGGUU | 2495 |
| 682 | CCUUCCGU G ACCUUGGC | 916 | GCCAAGGU GGAGGAAACUCC CU UCAAGGACAUCGUCCGGG ACGGAAGG | 2496 |
| 688 | GUGACCUU G GCCGCCUC | 917 | GAGGCGGC GGAGGAAACUCC CU UCAAGGACAUCGUCCGGG AAGGUCAC | 2497 |
| 699 | CGCCUCAU G ACACUCUA | 918 | UAGAGUGU GGAGGAAACUCC CU UCAAGGACAUCGUCCGGG AUGAGGCG | 2498 |
| 742 | UGCCCACU G AGGCCCUG | 919 | CAGGGCCU GGAGGAAACUCC CU UCAAGGACAUCGUCCGGG AGUGGGCA | 2499 |
| 744 | CCCACUGA G GCCCUGGC | 920 | GCCAGGGC GGAGGAAACUCC CU UCAAGGACAUCGUCCGGG UCAGUGGG | 2500 |
| 750 | GAGGCCCU G GCCCCCCU | 921 | AGGGGGGC GGAGGAAACUCC CU UCAAGGACAUCGUCCGGG AGGGCCUC | 2501 |
| 777 | GACUACCU G AGGCUCAA | 922 | UUGAGCCU GGAGGAAACUCC CU UCAAGGACAUCGUCCGGG AGGUACUG | 2502 |
| 779 | GUACCUGA G GCUCAACG | 923 | CGUUGAGC GGAGGAAACUCC CU UCAAGGACAUCGUCCGGG UCAGGUAC | 2503 |
| 787 | GGCUCAAC G ACAACCCC | 924 | GGGGUUGU GGAGGAAACUCC CU UCAAGGACAUCGUCCGGG GUUGAGCC | 2504 |
| 797 | CAACCCCU G GGUGUGUG | 925 | CACACACC GGAGGAAACUCC CU UCAAGGACAUCGUCCGGG AGGGGUUG | 2505 |
| 798 | AACCCCUG G GUGUGUGA | 926 | UCACACAC GGAGGAAACUCC CU UCAAGGACAUCGUCCGGG CAGGGGUU | 2506 |
| 805 | GGGUGUGU G ACUGCCGG | 927 | CCGGCAGU GGAGGAAACUCC CU UCAAGGACAUCGUCCGGG ACACACCC | 2507 |
| 812 | UGACUGCC G GGCACGCC | 928 | GGCGUGCC GGAGGAAACUCC CU UCAAGGACAUCGUCCGGG GGCAGUCA | 2508 |
| 813 | GACUGCCG G GCACGCCC | 929 | GGGCGUGC GGAGGAAACUCC CU UCAAGGACAUCGUCCGGG CGGCAGUC | 2509 |
| 827 | CCCACUCU G GGCCUGGC | 930 | GCCAGGCC GGAGGAAACUCC CU UCAAGGACAUCGUCCGGG AGAGUGGG | 2510 |
| 828 | CCACUCUG G GCCUGGCU | 931 | AGCCAGGC GGAGGAAACUCC CU UCAAGGACAUCGUCCGGG CAGAGUGG | 2511 |
| 833 | CUGGGCCU G GCUGCAGA | 932 | UCUGCAGC GGAGGAAACUCC CU UCAAGGACAUCGUCCGGG AGGCCCAG | 2512 |
| 840 | UGGCUGCA G AAGUUCCG | 933 | CGGAACUU GGAGGAAACUCC CU UCAAGGACAUCGUCCGGG UGCAGCCA | 2513 |
| 850 | AGUUCCGC G GCUCCUCC | 934 | GGAGGAGC GGAGGAAAUCCC CU UCAAGGACAUCGUCCGGG GCGGAACU | 2514 |
| 862 | CCUCCUCC G AGGUGCCC | 935 | GGGCACCU GGAGGAAACUCC CU UCAAGGACAUCGUCCGGG GGAGGAGG | 2515 |
| 864 | UCCUCCGA G GUGCCCUG | 936 | CAGGGCAC GGAGGAAACUCC CU UCAAGGACAUCGUCCGGG UCGGAGGA | 2516 |
| 891 | CAACGCCU G GCUGGCCG | 937 | CGGCCAGC GGAGGAAACUCC CU UCAAGGACAUCGUCCGGG AGGCGUUG | 2517 |
| 895 | GCCUGGCU G GCCGUGAC | 938 | GUCACGGC GGAGGAAACUCC CU UCAAGGACAUCGUCCGGG AGCCAGGC | 2518 |
| 901 | CUGGCCGU G ACCUCAAA | 939 | UUUGAGGU GGAGGAAACUCC CU UCAAGGACAUCGUCCGGG ACGGCCAG | 2519 |
| 925 | CUGCCAAU G ACCUGCAG | 940 | CUGCAGGU GGAGGAAACUCC CU UCAAGGACAUCGUCCGGG AUUGGCAG | 2520 |
| 933 | GACCUGCA G GGCUGCGC | 941 | GCGCAGCC GGAGGAAACUCC CU UCAAGGACAUCGUCCGGG UGCAGGUC | 2521 |
| 934 | ACCUGCAG G CCUGCGCU | 942 | AGCGCAGC GGAGGAAACUCC CU UCAAGGACAUCGUCCGGG CUGCAGGU | 2522 |
| 945 | UGCGCUGU G GCCACCGG | 943 | CCGGUGGC GGAGGAAACUCC CU UCAAGGACAUCGUCCGGG ACAGCGCA | 2523 |
| 952 | UGGCCACC G GCCCUUAC | 944 | GUAAGGGC GGAGGAAACUCC CU UCAAGGACAUCGUCCGGG GGUGGCCA | 2524 |
| 971 | UCCCAUCU G GACCGGCA | 945 | UGCCGGUC GGAGGAAACUCC CU UCAAGGACAUCGUCCGGG AGAUGGGA | 2525 |
| 972 | CCCAUCUG G ACCGGCAG | 946 | CUGCCGGU GGAGGAAACUCC CU UCAAGGACAUCGUCCCGG CAGAUGGG | 2526 |
| 976 | UCUGGACC G GCAGGGCC | 947 | GGCCCUGC GGAGGAAACUCC CU UCAAGGACAUCGUCCGGG GGUCCAGA | 2527 |
| 980 | GACCGGCA G GGCCACCG | 948 | CGGUGGCC GGAGGAAACUCC CU UCAAGGACAUCGUCCGGG UGCCGGUC | 2528 |
| 981 | ACCGGCAG G GCCACCGA | 949 | UCGGUGGC GGAGGAAACUCC CU UCAAGGACAUCGUCCGGG CUGCCGGU | 2529 |
| 988 | GGGCCACC G AUGAGGAG | 950 | CUCCUCAU GGAGGAAACUCC CU UCAAGGACAUCGUCCGGG GGUGGCCC | 2530 |
| 991 | CCACCGAU G AGGAGCCG | 951 | CGGCUCCU GGAGGAAACUCC CU UCAAGGACAUCGUCCGGG AUCGGUGG | 2531 |
| 993 | ACCGAUGA G GAGCCGCU | 952 | AGCGGCUC GGAGGAAACUCC CU UCAAGGACAUCGUCCGGG UCAUCGGU | 2532 |
| 994 | CCGAUGAG G AGCCGCUG | 953 | CAGCGGCU GGAGGAAACUCC CU UCAAGGACAUCGUCCGGG CUCAUCGG | 2533 |
| 1002 | GAGCCGCU G GGGCUUCC | 954 | GGAAGCCC GGAGGAAACUCC CU UCAAGGACAUCGUCCGGG AGCGGCUC | 2534 |
| 1003 | AGCCGCUG G GGCUUCCC | 955 | GGGAAGCC GGAGGAAACUCC CU UCAAGGACAUCGUCCGGG CAGCGGCU | 2535 |
| 1004 | GCCGCUGG G GCUUCCCA | 956 | UGGGAAGC GGAGGAAACUCC CU UCAAGGACAUCGUCCGGG CCAGCGGC | 2536 |
| 1027 | GCCAGCCA G AUGCCGCU | 957 | AGCGGCAU GGAGGAAACUCC CU UCAAGGACAUCGUCCGGG UGGCUGGC | 2537 |
| 1036 | AUGCCGCU G ACAAGGCC | 958 | GGCCUUGU GGAGGAAACUCC CU UCAAGGACAUCGUCCGGG AGCGGCAU | 2538 |
| 1041 | GCUGACAA G GCCUCAGU | 959 | ACUGAGGC GGAGGAAACUCC CU UCAAGGACAUCGUCCGGG UUGUCAGC | 2539 |
| 1053 | UCAGUACU G GAGCCUGG | 960 | CCAGGCUC GGAGGAAACUCC CU UCAAGGACAUCGUCCGGG AGUACUGA | 2540 |
| 1054 | CAGUACUG G AGCCUGGA | 961 | UCCAGGCU GGAGGAAACUCC CU UCAAGGACAUCGUCCGGG CAGUACUG | 2541 |
| 1060 | UGGAGCCU G GAAGACCA | 962 | UGGUCUUC GGAGGAAACUCC CU UCAAGGACAUCGUCCGGG AGGCUCCA | 2542 |
| 1061 | GGAGCCUG G AAGACCAG | 963 | CUGGUCUU GGAGGAAACUCC CU UCAAGGACAUCGUCCGGG CAGGCUCC | 2543 |
| 1064 | GCCUGGAA G ACCAGCUU | 964 | AAGCUGGU GGAGGAAACUCC CU UCAAGGACAUCGUCCGGG UUCCAGGC | 2544 |
| 1074 | CCAGCUUC G GCAGGCAA | 965 | UUGCCUGC GGAGGAAACUCC CU UCAAGGACAUCGUCCGGG GAAGCUGG | 2545 |
| 1078 | CUUCGGCA G GCAAUGCG | 966 | CGCAUUGC GGAGGAAACUCC CU UCAAGGACAUCGUCCGGG UGCCGAAG | 2546 |
| 1089 | AAUGCGCU G AAGGGACG | 967 | CGUCCCUU GGAGGAAACUCC CU UCAAGGACAUCGUCCGGG AGCGCAUU | 2547 |
| 1092 | GCGCUGAA G GGACGCGU | 968 | ACGCGUCC GGAGGAAACUCC CU UCAAGGACAUCGUCCGGG UUCAGCGC | 2548 |
| 1093 | CGCUGAAG G GACGCGUG | 969 | CACGCGUC GGAGGAAACUCC CU UCAAGGACAUCGUCCGGG CUUCAGCG | 2549 |
| 1094 | GCUGAAGG G ACGCGUGC | 970 | GCACGCGU GGAGGAAACUCC CU UCAAGGACAUCGUCCGGG CCUUCAGC | 2550 |
| 1108 | UGCCGCCC G GUGACAGC | 971 | GCUGUCAC GGAGGAAACUCC CU UCAAGGACAUCGUCCGGG GGGCGGCA | 2551 |
| 1111 | CGCCCGGU G ACAGCCCG | 972 | CGGGCUGU GGAGGAAACUCC CU UCAAGGACAUCGUCCGGG ACCGGGCG | 2552 |
| 1122 | AGCCCGCC G GGCAACGG | 973 | CCGUUGCC GGAGGAAACUCC CU UCAAGGACAUCGUCCGGG GGCGGGCU | 2553 |
| 1123 | GCCCGCCG G GCAACGGC | 974 | GCCGUUGC GGAGGAAACUCC CU UCAAGGACAUCGUCCGGG CGGCGGGC | 2554 |
| 1129 | CGGGCAAC G GCUCUGGC | 975 | GCCAGAGC GGAGGAAACUCC CU UCAAGGACAUCGUCCGGG GUUGCCCG | 2555 |
| 1135 | ACGGCUCU G GCCCACGG | 976 | CCGUGGGC GGAGGAAACUCC CU UCAAGGACAUCGUCCGGG AGAGCCGU | 2556 |
| 1142 | UGGCCCAC G GCACAUCA | 977 | UGAUGUGC GGAGGAAACUCC CU UCAAGGACAUCGUCCGGG GUGGGCCA | 2557 |
| 1153 | ACAUCAAU G ACUCACCC | 978 | GGGUGAGU GGAGGAAACUCC CU UCAAGGACAUCGUCCGGG AUUGAUGU | 2558 |
| 1165 | CACCCUUU G GGACUCUG | 979 | CAGAGUCC GGAGGAAACUCC CU UCAAGGACAUCGUCCGGG AAAGGGUG | 2559 |
| 1166 | ACCCUUUG G GACUCUGC | 980 | GCAGAGUC GGAGGAAACUCC CU UCAAGGACAUCGUCCGGG CAAAGGGU | 2560 |
| 1167 | CCCUUUGG G ACUCUGCC | 981 | GGCAGAGU GGAGGAAACUCC CU UCAAGGACAUCGUCCGGG CCAAAGGG | 2561 |
| 1177 | CUCUGCCU G GCUCUGCU | 982 | AGCAGAGC GGAGGAAACUCC CU UCAAGGACAUCGUCCGGG AGGCAGAG | 2562 |
| 1186 | GCUCUGCU G AGCCCCCG | 983 | CGGGGGCU GGAGGAAACUCC CU UCAAGGACAUCGUCCGGG AGCAGAGC | 2563 |
| 1208 | UGCAGUGC G GCCCGAGG | 984 | CCUCGGGC GGAGGAAACUCC CU UCAAGGACAUCGUCCGGG GCACUGCA | 2564 |
| 1213 | UGCGGCCC G AGGGCUCC | 985 | GGAGCCCU GGAGGAAACUCC CU UCAAGGACAUCGUCCGGG GGGCCGCA | 2565 |
| 1215 | CGGCCCGA G GGCUCCGA | 986 | UCGGAGCC GGAGGAAACUCC CU UCAAGGACAUCGUCCGGG UCGGGCCG | 2566 |
| 1216 | GGCCCGAG G GCUCCGAG | 987 | CUCGGAGC GGAGGAAACUCC CU UCAAGGACAUCGUCCGGG CUCGGGCC | 2567 |
| 1222 | AGGGCUCC G AGCCACCA | 988 | UGGUGGCU GGAGGAAACUCC CU UCAAGGACAUCGUCCGGG GGAGCCCU | 2568 |
| 1231 | AGCCACCA G GGUUCCCC | 989 | GGGGAACC GGAGGAAACUCC CU UCAAGGACAUCGUCCGGG UGGUGGCU | 2569 |
| 1232 | GCCACCAG G GUUCCCCA | 990 | UGGGGAAC GGAGGAAACUCC CU UCAAGGACAUCGUCCGGG CUGGUGGC | 2570 |
| 1245 | CCCACCUC G GGCCCUCG | 991 | CGAGGGCC GGAGGAAACUCC CU UCAAGGACAUCGUCCGGG GAGGUGGG | 2571 |
| 1246 | CCACCUCG G GCCCUCGC | 992 | GCGAGGGC GGAGGAAACUCC CU UCAAGGACAUCGUCCGGG CGAGGUGG | 2572 |
| 1256 | CCCUCGCC G GAGGCCAG | 993 | CUGGCCUC GGAGGAAACUCC CU UCAAGGACAUCGUCCGGG GGCGAGGG | 2573 |
| 1257 | CCUCGCCG G AGGCCAGG | 994 | CCUGGCCU GGAGGAAACUCC CU UCAAGGACAUCGUCCGGG CGGCGAGG | 2574 |
| 1259 | UCGCCGGA G GCCAGGCU | 995 | AGCCUGGC GGAGGAAACUCC CU UCAAGGACAUCGUCCGGG UCCGGCGA | 2575 |
| 1264 | GGAGGCCA G GCUGUUCA | 996 | UGAACAGC GGAGGAAACUCC CU UCAAGGACAUCGUCCGGG UGGCCUCC | 2576 |
| 1278 | UCACGCAA G AACCGCAC | 997 | GUGCGGUU GGAGGAAACUCC CU UCAAGGACAUCGUCCGGG UUGCGUGA | 2577 |
| 1305 | UGCCGUCU G GGCCAGGC | 998 | GCCUGGCC GGAGGAAACUCC CU UCAAGGACAUCGUCCGGG AGACGGCA | 2578 |
| 1306 | GCCGUCUG G GCCAGGCA | 999 | UGCCUGGC GGAGGAAACUCC CU UCAAGGACAUCGUCCGGG CAGACGGC | 2579 |
| 1311 | CUGGGCCA G GCAGGCAG | 1000 | CUGCCUGC GGAGGAAACUCC CU UCAAGGACAUCGUCCGGG UGGCCCAG | 2580 |
| 1315 | GCCAGGCA G GCAGCGGG | 1001 | CCCGCUGC GGAGGAAACUCC CU UCAAGGACAUCGUCCGGG UGCCUGGC | 2581 |
| 1321 | CAGGCAGC G GGGGUGGC | 1002 | GCCACCCC GGAGGAAACUCC CU UCAAGGACAUCGUCCGGG GCUGCCUG | 2582 |
| 1322 | AGGCAGCG G GGGUGGCG | 1003 | CGCCACCC GGAGGAAACUCC CU UCAAGGACAUCGUCCGGG CGCUGCCU | 2583 |
| 1323 | GGCAGCGG G GGUGGCGG | 1004 | CCGCCACC GGAGGAAACUCC CU UCAAGGACAUCGUCCGGG CCGCUGCC | 2584 |
| 1324 | GCAGCGGG G GUGGCGGG | 1005 | CCCGCCAC GGAGGAAACUCC CU UCAAGGACAUCGUCCGGG CCCGCUGC | 2585 |
| 1327 | GCGGGGGU G GCGGGACU | 1006 | AGUCCCGC GGAGGAAACUCC CU UCAAGGACAUCGUCCGGG ACCCCCGC | 2586 |
| 1330 | GGGGUGGC G GGACUGGU | 1007 | ACCAGUCC GGAGGAAACUCC CU UCAAGGACAUCGUCCGGG GCCACCCC | 2587 |
| 1331 | GGGUGGCG G GACUGGUG | 1008 | CACCAGUC GGAGGAAACUCC CU UCAAGGACAUCGUCCGGG CGCCACCC | 2588 |
| 1332 | GGUGGCGG G ACUGGUGA | 1009 | UCACCAGU GGAGGAAACUCC CU UCAAGGACAUCGUCCGGG CCGCCACC | 2589 |
| 1336 | GCGGGACU G GUGACUCA | 1010 | UGAGUCAC GGAGGAAACUCC CU UCAAGGACAUCGUCCGGG AGUCCCGC | 2590 |
| 1339 | GGACUGGU G ACUCAGAA | 1011 | UUCUGAGU GGAGGAAACUCC CU UCAAGGACAUCGUCCGGG ACCAGUCC | 2591 |
| 1345 | GUGACUCA G AAGGCUCA | 1012 | UGAGCCUU GGAGGAAACUCC CU UCAAGGACAUCGUCCGGG UGAGUCAC | 2592 |
| 1348 | ACUCAGAA G GCUCAGGU | 1013 | ACCUGAGC GGAGGAAACUCC CU UCAAGGACAUCGUCCGGG UUCUGAGU | 2593 |
| 1354 | AAGGCUCA G GUGCCCUA | 1014 | UAGGGCAC GGAGGAAACUCC CU UCAAGGACAUCGUCCGGG UGAGCCUU | 2594 |
| 1392 | ACCCCCCU G GGCCUGGC | 1015 | GCCAGGCC GGAGGAAACUCC CU UCAAGGACAUCGUCCGGG AGGGGGGU | 2595 |
| 1393 | CCCCCCUG G GCCUGGCG | 1016 | CGCCAGGC GGAGGAAACUCC CU UCAAGGACAUCGUCCGGG CAGGGGGG | 2596 |
| 1398 | CUGGGCCU G GCGCUGGU | 1017 | ACCAGCGC GGAGGAAACUCC CU UCAAGGACAUCGUCCGGG AGGCCCAG | 2597 |
| 1404 | CUGGCGCU G GUGCUGUG | 1018 | CACAGCAC GGAGGAAACUCC CU UCAAGGACAUCGUCCGGG AGCGCCAG | 2598 |
| 1412 | GGUGCUGU G GACAGUGC | 1019 | GCACUGUC GGAGGAAACUCC CU UCAAGGACAUCGUCCGGG ACAGCACC | 2599 |
| 1413 | GUGCUGUG G ACAGUGCU | 1020 | AGCACUGU GGAGGAAACUCC CU UCAAGGACAUCGUCCGGG CACAGCAC | 2600 |
| 1423 | CAGUGCUU G GGCCCUGC | 1021 | GCAGGGCC GGAGGAAACUCC CU UCAAGGACAUCGUCCGGG AAGCACUG | 2601 |
| 1424 | AGUGCUUG G GCCCUGCU | 1022 | AGCAGGGC GGAGGAAACUCC CU UCAAGGACAUCGUCCGGG CAAGCACU | 2602 |
| 1433 | GCCCUGCU G ACCCCCAG | 1023 | CUGGGGGU GGAGGAAACUCC CU UCAAGGACAUCGUCCGGG AGCAGGGC | 2603 |

Input Sequence = AF283463. Cut Site = G/.
Arm Length = 8. Core Sequence = GGAGGAAACUCC CU UCAAGGACAUCGUCCGGG
AF283463 (Homo sapiens Nogo receptor mRNA, complete cds.; 1441 bp)

What we claim is:

1. A nucleic acid molecule which down regulates expression of a receptor for a neurite growth inhibitor.

2. A nucleic acid molecule of claim 1, wherein the gene encoding said neurite growth inhibitor receptor gene is a NOGO receptor gene.

3. The nucleic acid of claim 1, wherein said nucleic acid molecule is adapted for use to treat conditions selected from the group consisting of CNS injury, spinal cord injury, and cerebrovascular accident.

4. The nucleic acid molecule of claim 1 or claim 2, wherein said nucleic acid molecule is an enzymatic nucleic acid molecule having at least one binding arm.

5. The nucleic acid molecule of claim 4, wherein said enzymatic nucleic acid molecule has an endonuclease activity to cleave RNA encoded by a NOGO receptor gene.

6. The nucleic acid of claim 4, wherein one or more binding arms of the enzymatic nucleic acid molecule comprises a sequence complementary to a sequence selected from the group consisting of SEQ ID NOs. 1-1023.

7. An enzymatic nucleic acid molecule comprising a sequence selected from the group consisting of SEQ ID NOs. 1024-2603.

8. The nucleic acid molecule of claim 1, wherein said nucleic acid molecule is an antisense nucleic acid molecule.

9. An antisense nucleic acid molecule comprising a sequence complementary to a sequence selected from the group consisting of SEQ ID NOs. 1-1023.

10. The enzymatic nucleic acid molecule of claim 4, wherein said enzymatic nucleic acid molecule is in a hammerhead (HH) motif.

11. The enzymatic nucleic acid molecule of claim 4, wherein said enzymatic nucleic acid molecule is in a hairpin, hepatitis Delta virus, group I intron, VS nucleic acid, amberzyme, zinzyme or RNAse P nucleic acid motif.

12. The enzymatic nucleic acid molecule of claim 11, wherein said zinzyme motif comprises a sequence selected from the group consisting of SEQ ID NOs. 1572-1815.

13. The enzymatic nucleic acid molecule of claim 11, wherein said amberzyme motif comprises a sequence selected from the group consisting of SEQ ID NOs. 2176-2603.

14. The enzymatic nucleic acid molecule of claim 4, wherein said enzymatic nucleic acid molecule is in a NCH motif.

15. The enzymatic nucleic acid molecule of claim 4, wherein said enzymatic nucleic acid molecule is in a G-cleaver motif.

16. The enzymatic nucleic acid molecule of claim 4, wherein said enzymatic nucleic acid molecule is a DNAzyme.

17. The nucleic acid molecule of claim 2, wherein said nucleic acid molecule comprises between 12 and 100 bases complementary to the RNA of NOGO receptor gene.

18. The nucleic acid molecule of claim 2, wherein said nucleic acid molecule comprises between 14 and 24 bases complementary to the RNA of NOGO receptor gene.

19. The nucleic acid molecule of claim 1, wherein said nucleic acid molecule is chemically synthesized.

20. The nucleic acid molecule of claim 1, wherein said nucleic acid molecule comprises at least one 2′-sugar modification.

21. The nucleic acid molecule of claim 1, wherein said nucleic acid molecule comprises at least one nucleic acid base modification.

22. The nucleic acid molecule of claim 1, wherein said nucleic acid molecule comprises at least one phosphate backbone modification.

23. A mammalian cell including the nucleic acid molecule of claim 1.

24. The mammalian cell of claim 23, wherein said mammalian cell is a human cell.

25. A method of reducing NOGO receptor activity in a cell, comprising the step of contacting said cell with the nucleic acid molecule of claim 2, under conditions suitable for said inhibition.

26. A method of treatment of a patient having a condition associated with the level of NOGO receptor, comprising contacting cells of said patient with the nucleic acid molecule of claim 2, under conditions suitable for said treatment.

27. The method of claim 26 further comprising the use of one or more drug therapies under conditions suitable for said treatment.

28. A method of cleaving RNA of NOGO receptor gene contacting the nucleic acid molecule of claim 2 with said RNA under conditions suitable for the cleavage of said RNA.

29. The method of claim 28, wherein said cleavage is carried out in the presence of a divalent cation.

30. The method of claim 29, wherein said divalent cation is Mg²⁺.

31. The nucleic acid molecule of claim 1, wherein said nucleic acid comprises a cap structure, wherein the cap structure is at the 5′-end, or 3′-end, or both the 5′-end and the 3′-end.

32. The enzymatic nucleic acid molecule of claim 10, wherein said hammerhead motif comprises a sequence selected from the group consisting of SEQ ID NOs. 1024-1123.

33. The enzymatic nucleic acid molecule of claim 14, wherein said NCH motif comprises a sequence selected from the group consisting of SEQ ID NOs. 1124-1571.

34. The enzymatic nucleic acid molecule of claim 16, wherein said DNAzyme comprise a sequence selected from the group consisting of SEQ ID NOs. 1816-2175.

35. The method of claim 25, wherein said nucleic acid molecule is in a hammerhead motif.

36. The method of claim 25, wherein said nucleic acid molecule is a DNAzyme.

37. An expression vector comprising a nucleic acid sequence encoding at least one nucleic acid molecule of claim 1 in a manner which allows expression of the nucleic acid molecule.

38. A mammalian cell including an expression vector of claim 37.

39. The mammalian cell of claim 38, wherein said mammalian cell is a human cell.

40. The expression vector of claim 37, wherein said nucleic acid molecule is in a hammerhead motif.

41. The expression vector of claim 37, wherein said expression vector further comprises a sequence for an antisense nucleic acid molecule complementary to the RNA of NOGO receptor gene.

42. The expression vector of claim 37, wherein said expression vector comprises a nucleic acid sequence encoding two or more of said nucleic acid molecules, which may be the same or different.

43. The expression vector of claim 42, wherein said expression vector comprises a sequence encoding antisense nucleic acid molecule complementary to the RNA of NOGO receptor gene.

44. A method for treatment of conditions selected from the group consisting of CNS injury and cerebrovascular accident comprising the step of administering to a patient the nucleic acid molecule of claim 1 under conditions suitable for said treatment.

45. The method of claim 44, wherein said treatment of CNS injury is treatment of spinal cord injury.

46. A method for treatment of conditions selected from the group consisting of CNS injury and cerebrovascular accident comprising the step of administering to a patient the antisense nucleic acid molecule of claim 9 under conditions suitable for said treatment.

47. The method of claim 44, wherein said nucleic acid molecule is in a hammerhead motif.

48. The method of claim 44, wherein said method further comprises administering to said patient one or more other therapies.

49. The nucleic acid molecule of claim 1, wherein said nucleic acid molecule comprises at least five ribose residues, at least ten 2′-O-methyl modifications, and a 3′-end modification.

50. The nucleic acid molecule of claim 49, wherein said nucleic acid molecule further comprises phosphorothioate linkages on at least three of the 5′ terminal nucleotides.

51. The nucleic acid molecule of claim 49, wherein said 3′-end modification is 3′-3′ inverted abasic moiety.

52. The enzymatic nucleic acid molecule of claim 16, wherein said DNAzyme comprises at least ten 2′-O-methyl modifications and a 3′-end modification.

53. The enzymatic nucleic acid molecule of claim 52, wherein said DNAzyme further comprises phosphorothioate linkages on at least three of the 5′ terminal nucleotides.

54. The enzymatic nucleic acid molecule of claim 52, wherein said 3′-end modification is 3′-3′ inverted abasic moiety.
